(19) United States
(12) Patent Application Publication
(10) Pub. No.: US 2024/0139724 A1
(43) Pub. Date: May 2, 2024

(54) DEVICES AND METHODS FOR MANIPULATION AND CONCENTRATION OF PARTICLES AND LARGE MOLECULES IN A DISPOSABLE FILTER TIP

(71) Applicant: INNOVAPREP LLC, Drexel, MO (US)

(72) Inventors: Andrew E. Page, Smithton, MO (US); David Goad, Stillwater, OK (US); Steven D. Graham, Drexel, MO (US)

(21) Appl. No.: 18/384,345

(22) Filed: Oct. 26, 2023

Related U.S. Application Data
(60) Provisional application No. 63/419,682, filed on Oct. 26, 2022.

(51) Int. Cl. B01L 3/02; A61M 5/315; G01N 35/10

(52) U.S. Cl. CPC B01L 3/0217; A61M 5/31565; B01L 3/0275; G01N 35/1004; B01L 2300/0681; B01L 2400/0487; B01L 2400/0622; B01L 2400/0644; G01N 2035/1006

(57) ABSTRACT

Highly efficient and rapid filtration-based concentration devices, systems and methods are disclosed with a rotary distribution valve connected to a syringe pump which maintains control and flow of various fluids to a sample through a retentate port.

This U.S. Patent application claims priority to U.S. Provisional Patent Application Ser. No. 63/419,682, filed Oct. 26, 2022; the contents of which are hereby incorporated by reference herein in their entirety into this disclosure.

### BACKGROUND OF THE SUBJECT DISCLOSURE

### Field of the Subject Disclosure

The subject disclosure relates generally to the field of sample preparation. More particularly, the subject disclosure relates to devices and methods for automated manipulation and concentration of particles and large molecules in a disposable filter tip for improving the quality of the sample and enhancing the sensitivity of subsequent analysis methods and the reliability of downstream processes using the particles and large molecules.

### Background of the Subject Disclosure

Many biotechnology, life sciences, environmental sciences and other scientific and medical fields have a significant need for approaches for manipulating particles, both biological and non-biological, and large molecules prior to performing downstream processes and analytical methods. These approaches can include concentration, washing, clarifying, performing buffer exchanges, labeling, dying, capturing, lysis, and other manipulations.

Many of these manipulations are traditionally performed using centrifugation. Centrifugal force is used for the separation of mixtures according to differences in the density of the individual components present in the mixture. This force separates a mixture forming a pellet of relatively dense material at the bottom of the tube. The remaining solution, referred to as the supernate or supernatant liquid, may then be carefully decanted from the tube without disturbing the pellet, or withdrawn using a Pasteur pipette. The rate of centrifugation is specified by the acceleration applied to the sample, and is typically measured in revolutions per minute (RPM) or g-forces. The particle settling velocity in centrifugation is a function of the particle's size and shape, centrifugal acceleration, the volume fraction of solids present, the density difference between the particle and the liquid, and viscosity of the liquid.

Problems with the centrifugation technique limit its applicability. The settling velocity of particles in the micron size range is quite low, and even lower in the sub-micron size range. Consequently, centrifugal concentration of these particles takes several minutes to many hours. The actual time varies depending on the volume of the sample, the equipment used, and the skill of the operator. The nature of centrifugation techniques and of the devices used to perform centrifugation requires a skilled operator, thus making automation and integration into other systems difficult.

Centrifugation techniques are tedious in that they are normally made up of multiple steps each requiring a high level of concentration from the operator. It is common in most microbiology laboratories to process large numbers of samples by centrifugation on a daily basis. The potential for human error is high due to the tedious nature; and as stated earlier automation of these techniques is difficult and costly.

Other concentration techniques have been explored and primarily fall into three technology groups—microfluidic/electrophoretic based, filtration based, and capture based. Each of these techniques has advantages and disadvantages.

Traditional flat filtration methodology is used to capture particles from a liquid onto a flat filter, usually supported by a screen or fritted substrate. Many different methods of filtration exist, but all aim to attain the separation of two or more substances. This is achieved by some form of interaction between the substance or objects to be removed and the filter. The substance that is to pass through the filter must be a fluid, i.e., a liquid or gas. The simplest method of filtration is to pass a solution of a solid and fluid through a porous interface so that the solid is trapped, while the fluid passes through. This principle relies upon the size difference between the particles contained in the fluid, and the particles making up the solid. In the laboratory, this if often done using a Büchner funnel with a filter paper that serves as the porous barrier.

One disadvantage of the physical barrier method of filtration is that the substance being filtered from the fluid will clog the channels through the filter over time. The resistance to flow through the filter becomes greater and greater over time as, for example, a vacuum cleaner bag. Accordingly, methods have been developed to prevent this from happening. Most such methods involve replacing the filter; however, if the filter is needed for a continuous process this need for replacement is highly problematic. Scraping and in-situ cleaning mechanisms may be used, but these can be unnecessarily complex and expensive.

In one example, bacteria may be removed from water by passing them through a filter supported in a Buchner funnel to trap the bacteria on the flat filter. Aerosol particles containing biological materials can also be trapped in the same way. For analysis, the trapped materials are often re-suspended in a known volume of liquid. This allows back-calculation of the original aerosol concentration. One method validated by the Edgewood Chemical Biological Center uses 47 mm glass-fiber filters to capture reference samples for biological analysis. The bacteria are extracted by soaking the filters overnight in 20 mL of buffered saline solution, then vortexed for 3 minutes to disrupt the filter material completely. Subsamples or aliquots of these suspensions are then provided for analysis by viable culture, PCR, or other methods.

Other technologies for concentration of biological particulate matter exist. Sandia National Laboratories, Massachusetts Institute of Technology, and other organizations have developed microfluidic devices that separate and concentrate particles by dielectrophoresis or electrophoresis. These units use microchannels and electric fields to move or collect particles. Sandia has also developed a system that concentrates particles at the interface between two immiscible liquids. Immunomagnetic particles are commercially available for use in the separation and concentration of bacteria.

Various methods exist for concentrating organisms in liquids prior to detection. Historically, the most common method is to enrich the sample in nutrient broth and then cultivate an aliquot of the broth on an agar plate. The biggest disadvantage of this method is the time requirement. It normally takes five to seven days before organisms can be enumerated on the plates. Additionally, certain organisms, viruses, and other particles can be difficult or impossible to enrich using current approaches. Other concentration methods include various filtration-based methods, adsorption-elution, immunocapture, flocculation, and centrifugation. It is problematic that to date no automated methods have been developed that can rapidly concentrate a large volume of water into a very small sample volume and do this task efficiently. In fact, most of these methods fail in each of these areas, most notably efficiency of concentration, delivered concentration factor, and ease of use.

A considerable amount of research has been performed using hollow fiber ultrafiltration to concentrate bacteria, viruses, and protozoa from large volumes of water. Most of the methods described are not automated. Generally, these systems are capable of concentrating 10 to 100 L water into 100 to 500 mL of concentrated sample; however, it is further problematic that none of the demonstrated technologies provides concentration into volumes of less than 100 mL. Even this volume is much larger than desired for the best possible detection when the concentrator systems are coupled with downstream detection apparatus. This means that a costly and time-consuming second manual concentration step is required to bring the final sample to the desired volume.

The alternative concentration systems described above, although automated, do not provide significant advantages over traditional centrifugation for many laboratories, including microbiology, biotechnology, and clinical biology laboratories. These laboratories require a high level of certainty that sample to sample contamination does not take place. The alternative, automated concentration systems, have significant fluidics that samples are exposed to and in many cases it is, at best, costly and, at worst, impossible to replace these fluidics lines between samples.

The potential for carryover of particles of interest or signatures from one sample to another and the potential for growth of bacteria within the system fluidics significantly limit their applicability to clinical laboratories. In general, microbiology and biotechnology laboratories have adopted the use of disposable components in nearly all work.

Spin columns, or centrifugal filters as they are often called, that contain an ultrafilter or microfilter type membrane filters and can be placed into a centrifuge or in some instances use positive pressure to drive the liquid through are a relatively new device that is now seeing widespread use in these laboratories. These centrifugal spin columns overcome the contamination issues associated with other concentration systems and also overcome many of the issues associated with using centrifugation to concentration biological materials; however, the spin columns are costly, due to their complexity, and still require significant manual manipulation and pipetting during operation. A high skill level is also required for their use. Additionally, centrifugal filters have limited process volume based on the internal volume available in the device. Thus approximately 70 mL is the general upper volume that can be processed. Finally, all manipulations require one or more manual steps to be performed by the user.

### SUMMARY OF THE SUBJECT DISCLOSURE

A device for automated manipulation and concentration of particles and large molecules with a disposable fluid path that is capable of processing relatively large volumes of liquids would have significant applicability to clinical diagnostics and microbiology and biotechnology laboratories.

The present Applicant (InnovaPrep) holds multiple patents related to methods and devices for concentration of biological particles from liquid samples. These systems use disposable filter tips, or Concentrating Pipette Tips, to capture target particles which are subsequently eluted using wet foam elution. While these devices and methods provide significant utility for concentrating bacteria, viruses and other small particles, the patents related to these products do not describe methods or devices in sufficient detail to enable those skilled in the art to perform or produce devices or develop methods for more complex operations such as addition of sample amendments through the tip, filter blocking, filter washes, filter fouling reduction steps, and lysis steps using the disposable tips or instruments for operating the disposable tips.

These operations for enabling addition of sample amendments, performing filter blocking, filter washes, filter fouling reduction steps, labeling, dying, capturing, and lysis steps within a disposable filter tip are not contemplated in the current art and are important for overcoming limitations of the current art and for enabling additional required sample preparation steps within the context of particle analysis and detection.

While the previously disclosed Concentrating Pipette technologies have provided concentration approaches for certain applications, its application use has been limited by performance and throughput deficiencies. Fouling of the membrane filters during sample processing has severely limited use for certain applications and sample matrices. Low recovery efficiency, particularly with complex sample matrices high in natural organic matter (NOM), has also limited use of these technologies for some targets. These previously described technologies lack provision for performing complex sample addition, membrane filter blocking, fouling reduction, or wash steps and thus suffer from significant fouling, co-concentration of inhibitors, and low particle recovery when processing complex matrices. Finally, low sample processing rates, caused by fouling, coupled with the single-plex and hands-on nature of these previously described approaches make use in large commercial laboratories impractical.

Automation and multiplexing of existing Concentrating Pipette technologies is a possibility for overcoming throughput deficiencies, but it fails to address the long run times resulting from fouling in many applications. As an example, for wastewater influent samples the initial processing rates with these previously described, and now commercialized systems, drop very quickly from over 150 mL/min to less than 10 mL/min within seconds of initiating sample processing, and this drop in flow rate significantly increases sample run times. For example, 50 mL clean water runs nominally take 30 seconds versus 3 minutes or more for wastewater influent samples. As sample volumes increase the issue becomes worse, with 100 mL clean water samples taking about 1 minute versus nearly 10 minutes for wastewater influent samples.

Membrane fouling is also problematic because it effectively reduces the membrane cut point, causing NOM, humic materials, proteins, polysaccharides, and other fouling materials, that based solely on size should pass through the membrane pores, to be retained at an accelerating rate. Increased retention of these materials presents multiple issues. Accumulated material, like NOM, often form a difficult to remove sticky surface inside hollow fibers that can trap target particles and decrease recovery efficiency. In addition, some of these materials, such as NOM and humic materials, are well known to inhibit downstream nucleic acid purification and PCR, and is co-concentrated with the target resulting in higher concentrations in eluted samples, exacerbating downstream inhibition issues. Longer run times, losses, and inhibition associated with fouling ultimately reduce both sample throughput and method sensitivity.

The present subject disclosure addresses the problem outlined and advances the art by providing a highly efficient filtration-based particle and large molecule concentration and manipulation system in which the sample matrix and target materials only come into contact with a disposable filter tip. The described disposable filter tip and methods of processing samples through the tip and eluting a concentrated sample is disclosed in earlier patent applications submitted by the present Applicant (InnovaPrep LLC). The disposable tip has been termed the Concentrating Pipette Tip (CPT) by InnovaPrep. The newly disclosed device and method advance significantly upon the earlier concepts by enabling a number of complex operations that provide for improved capabilities for processing samples; providing improved quality to processed and concentrated samples.

More specifically the disclosed system is capable of increasing sample volumes processed, improve concentration efficiencies and reduce co-concentrated inhibitors. Further, the system enables onboard lysis and bypass of currently necessary downstream extraction protocols, or integration with those processes—thereby enabling end-to-end automation of concentration, extraction, and purification of target particles.

The device and methods described here enable end-to-end automated systems for replacement of continuous flow and low-, high- and ultra-speed centrifugation, as well as centrifugal and tangential flow filtration in the laboratory. Cell washing, concentration/staining/labeling for cytometry and microscopy, purification and concentration of circulating tumor cells, microbes, viruses, bacteriophages, proteins, extracellular vesicles, exosomes, nucleic acids, spores, microplastics, and other particles and large molecules will be possible with the described device and methods.

### BRIEF DESCRIPTION OF THE DRAWINGS

FIGS. 1A and 1B show a concentrating pipette tip (CPT), according to an exemplary embodiment of the present subject disclosure.

FIGS. 2A and 2B show a similar configuration for a hollow fiber filter that will not allow air to pass through, according to an exemplary embodiment of the present subject disclosure.

FIG. 3 shows an alternative configuration for connection of a concentrating pipette tip (CPT) to the concentrator unit, according to an exemplary embodiment of the present subject disclosure.

FIG. 4 shows a CPT including an annular configuration for connection to the concentrating unit, according to an exemplary embodiment of the present subject disclosure.

FIG. 5 shows a CPT having pin type connectors, according to an exemplary embodiment of the present subject disclosure.

FIG. 6 shows a CPT including a primary male connector, according to an exemplary embodiment of the present subject disclosure.

FIG. 7 shows a CPT including a primary male connector, according to an exemplary embodiment of the present subject disclosure.

FIG. 8 shows a CPT including a primary male connector, according to an exemplary embodiment of the present subject disclosure.

FIGS. 9-11 show one configuration for a CPT, according to an exemplary embodiment of the present subject disclosure.

FIG. 12 shows another potential configuration for a CPT, according to an exemplary embodiment of the present subject disclosure.

FIG. 13 shows a configuration for a CPT with a flat porous surface dividing the tip into an upper portion and a lower portion with an opening at the lower end and a connector at the upper end, according to an exemplary embodiment of the present subject disclosure.

FIGS. 14A-C show another configuration for a CPT, according to an exemplary embodiment of the present subject disclosure.

FIG. 15 shows a concentrating unit gathering a sample through a CPT, according to an exemplary embodiment of the present subject disclosure.

FIG. 16 shows a method of using a concentrating unit having a CPT, according to an exemplary embodiment of the present subject disclosure.

FIGS. 17A and 17B show an alternate configuration for a CPT, according to an exemplary embodiment of the present subject disclosure.

FIGS. 18A and 18B show another concentrating unit for gathering a sample through a CPT, according to an exemplary embodiment of the present subject disclosure.

FIG. 19 shows a system for gathering a sample through a CPT, according to an exemplary embodiment of the present subject disclosure.

FIG. 20 shows an external view of a CPT having a flat filter, according to an exemplary embodiment of the present subject disclosure.

FIG. 21 shows a horizontal cross section of a CPT having a flat filter, according to an exemplary embodiment of the present subject disclosure.

FIG. 22 shows a vertical cross section of a CPT having a flat filter, according to an exemplary embodiment of the present subject disclosure.

FIGS. 23A and 23B show views of a CPT having a hollow fiber filter, according to an exemplary embodiment of the present subject disclosure.

FIG. 24 shows a vertical cross section of a CPT having a hollow fiber filter, according to an exemplary embodiment of the present subject disclosure.

FIG. 25 shows a horizontal cross section of a CPT having a hollow fiber filter, according to an exemplary embodiment of the present subject disclosure.

FIG. 26 shows an isometric view of a CPT having two filters, according to an exemplary embodiment of the present subject disclosure.

FIG. 27 shows an exploded view of a CPT having two filters, according to an exemplary embodiment of the present subject disclosure.

FIG. 28 shows a cross-sectional view of a CPT having two filters, according to an exemplary embodiment of the present subject disclosure.

FIG. 29 shows a system for performing sample manipulation and concentration using a CPT and a timed valve for wet foam elution metering, according to an exemplary embodiment of the present subject disclosure.

FIG. 30 shows a system for performing sample manipulation and concentration using a CPT and use of a syringe pump for wet foam elution metering, according to an exemplary embodiment of the present subject disclosure.

### DETAILED DESCRIPTION OF THE SUBJECT DISCLOSURE

The present subject disclosure is a device and method for performing complex sample manipulation and concentration steps within a disposable concentrating pipette tip. All conduits by which the disposable concentrating pipette tip attaches to the concentrator unit instrument are combined into a single connection point on the upper end of the concentrating pipette tip. The concentrating pipette tip (CPT) works with a system including a concentrator unit and a liquid sample. To operate the system, a new clean concentrating pipette tip is attached to the concentrator unit and the lower opening of the concentrating pipette tip is dipped into a liquid sample contained in an appropriate sample container and the concentrator unit is activated. The use of a new clean concentrating pipette tip ensures that there is no sample-to-sample carryover. The sample is then aspirated into the CPT where it comes into contact with a filter. The liquid is passed through the filter while particles and molecules larger than the filter pore size are captured and retained. When the entire sample has passed through the filter, removing the fluid and leaving the captured material, the lower opening of the tip is placed into an appropriate sample container and an elution fluid or foam is used to elute the captured material and dispense it in a reduced volume. Prior to aspirating the sample, it is possible to perform sample addition steps or membrane filter blocking steps to provide for improved processing and reduced losses of target materials. During processing of the sample it is then possible to perform additional sample additions, membrane filter blocking, wash steps or other sample processing steps. Further, high-frequency backpulsing can be performed to reduce membrane fouling. High-frequency backpulsing involves applying pressure to the permeate reservoir and thereby pushing small amounts of filtered sample or buffer through the membrane pores in a reverse direction to help in flow recovery. The backpulsing can be performed for very short periods of time at a high frequency to limit the reverse flow time periods and thus not negatively affect the system flow rate. The process is very similar to the oscillating tangential flow process, which will be well known by those skilled in the art, but is modified to allow for the process to be formed in a concentrating pipette tip with an open end.

In addition to backpulsing the described system is capable of injecting fluid into the upper end of the concentrating pipette tip retentate to enable a rapid tangential flow in the opposite direction to the normal liquid flow. This approach further improves fouling material disruption on the membrane surface and thereby improves sample flow rate.

High frequency backpulsing and a process termed oscillating tangential flow were previously described in the Applicant's prior filed patent application Ser. No. 17/222,951, entitled “Liquid to Liquid Biological Particle Concentrator with Disposable Fluid Path,” filed Apr. 5, 2022, which is incorporated by reference herein in its entirety. However, certain portions of the processes described within that application have been further improved upon in the system disclosed herein. The previous application described use of a metering pump to oscillate fluid back and forth within the concentrating pipette retentate which may create the potential for cross contamination of the concentrating pipette instrument. First, the oscillating flow requires that no check valve be used within the concentrating pipette retentate, as is shown in FIGS. 31 through 34 of the prior application. This is because the use of a check valve does not allow for flow in the direction of the instrument. Additionally, if the concentrating pipette tip does not contain a check, which allows for oscillating flow to be used, then fluid that potentially contains high concentrations of target particles, may enter into the concentrating pipette instrument, thus contaminating the instrument fluidics. For this reason, the current disclosure describes injection of fluid into the concentrating pipette tip from the instrument only, to create a reverse tangential flow within the retentate. In this way, a check valve can be used in the top of the tip retentate and the potential for cross contamination of the instrument is eliminated.

The Applicant's prior filed patent application Ser. No. 17/222,951, entitled “Liquid to Liquid Biological Particle Concentrator with Disposable Fluid Path,” filed Apr. 5, 2022, also describes use of injection of carbon dioxide into the permeate as the method for enabling high frequency backpulsing. While this approach may be used, it is further helpful to describe how to control the magnitude of the positive pressure within the permeate. This is because the negative pressure that exists within the concentrating pipette tip permeate during sample processing is variable based on the curve of the concentrating pipette tip flow rate versus the permeate negative pressure. Further this curve changes dynamically during sample processing as the membrane filter fouls and as fouling reversal is performed using the described processes. For these reasons, simply injecting gas under pressure into the concentrating pipette tip permeate may provide highly variable permeate pressure and thus potentially highly uncontrolled backpulsing. Described herein in this disclosure are three approaches for overcoming this deficiency.

A first approach for overcoming the described deficiencies of the previously described high-frequency backpressure system is use of a pressure transducer within the permeate flow path and a second pressure transducer fluidically connected to the backpressure reservoir that are used for monitoring the pressure within the permeate and within the pressure system. An algorithm is then utilized to determine the time period that a valve within the pressure system must be open to achieve the desired permeate backpressure. Alternatively, an algorithm may be utilized to determine what pressure to increase the pressure reservoir to in order to achieve the desired permeate backpressure when using a set pressure system control valve.

A second approach for overcoming the described deficiencies of the previously described high-frequency backpressure system is similar to the first described approach, in that it uses a permeate pressure transducer and a pressure reservoir transducer, but it is different in that the permeate pressure transducer is used to measure the achieved permeate pressure and to then close the permeate pressure control valve when a desired pressure is achieved. Using this approach a preset pressure is used for the permeate pressure reservoir and the valve connecting it to the permeate is opened and then when the permeate pressure setpoint is achieved, based on the permeate pressure transducer reading, the valve is closed.

A third approach for overcoming the described deficiencies of the previously described high-frequency backpressure system is use of a vent valve to allow for disconnection and connection of the permeate system to atmospheric pressure along with a pressure transducer fluidically connected to the backpressure reservoir. In this approach, the vent valve is used to first connect the permeate to atmospheric pressure immediately before opening the valve fluidically connecting the pressure reservoir to the concentrating pipette tip permeate. In this way, the pressure within the permeate is known to be at or near to atmospheric pressure before the pressure is applied. This allows the backpressure on the permeate to be tightly controlled using through pressure control of the permeate pressure reservoir along with the valve open time for the pressure reservoir valve.

These described approaches of fluid injection for reverse tangential flow and high-frequency permeate back pressure can be applied separately or together to reduce membrane filter fouling and improve flow rate within concentrating pipette tips. The steps can

Prior to dispensing the concentrated sample, it is also possible to perform wash steps, labeling steps, cell lysis, or other manipulation. Each of these described steps can be performed by pushing a small volume of fluid into the fiber lumen, drawing it out through the filter wall, or leaving it in the fiber lumen for a period of time prior to drawing it out, or alternatively by pushing the fluid down the lumen of the fiber and out the end of the concentrating pipette tip and into the sample. Further, these operations can be performed with the fluid injection at a range of flow rates, volumes, and temperatures to provide for improved performance of the described operations. After being dispensed, the concentrated sample may be further concentrated prior to analysis by immunomagnetic separation, electrophoretic or dielelectrophoretic separation techniques, or other microfluidic concentration techniques. In many instances these techniques are useful but are in general not possible with larger volumes or are prohibitively costly or slow when performed on large volumes. By rapidly performing an initial concentration with the CPT the sample volume is reduced to a volume that is more readily handled with these techniques.

It is further possible to apply additional sample preparation techniques to the concentrated sample once dispensed. Additional sample preparation techniques that may be applied include various methods of cell lysis, washing steps, inhibitor or interferent removal techniques, and labeling steps. Reduction of the sample volume prior to performing these techniques routinely improves the speed and efficiency, while reducing the cost of performing these techniques.

Analysis of the concentrated sample may be performed with any number of commonly used traditional analytical or microbiological analysis methods or rapid analysis techniques including rapid microbiological techniques. Analytical techniques of special interest include conventional methods of plating and enumeration, most probable number, immunoassay methods, polymerase chain reaction (PCR), electrochemical, microarray, flow cytometry, biosensors, lab-on-a-chip, and rapid growth-based detection technologies to name a few, as will be well known by those skilled in the art.

Microorganisms including pathogens and spoilage organisms may be concentrated from any number of beverages including fruit juices, vegetable juices, carbonated beverages, alcoholic beverages and from homogenates or liquid samples produced from solid foods. By concentrating large sample volumes in the range of 1 mL to 10 L or more prior to analysis it is possible to rapidly detect microorganisms at levels that were previously only detectable following lengthy culturing of a portion of the sample.

It is further possible to test samples resulting from manual swabbing of surfaces onto wetted swabs, pads, or pieces of filter material often taken for bioterrorism security monitoring. The samples are typically extracted into a volume of liquid resulting in a 2 to 20 mL volume initial sample. Samples like these may be quickly concentrated to much smaller volumes in the range of 4 to 400 μL such that agents may more easily be detected.

In still other aspects, samples may be concentrated for water sampling in search of bioterrorism agents, or in the interest of public health and safety, especially where a sample may contain target agent(s) that are thought to be a threat to the health of humans, animals or plants, causing societal disruption and economic harm. Agricultural products and livestock environments may also be evaluated by the instrumentalities herein disclosed.

Environmental studies that may also benefit from the present subject disclosure include many types of sampling and analysis that are performed for the field of environmental study, such as in assessing health effects through research regarding various materials in inhaled particulate matter with aerodynamic diameter below 2.5 microns (PM 2.5) or high altitude aerosol research where low quantities of particulate are collected and must be concentrated for study. These instrumentalities may benefit clean rooms where very low aerosol concentrations of aerosol particles are collected for monitoring that is aimed at source control.

Forensic sciences may also benefit from the present subject disclosure by allowing for detection of DNA collected from large surfaces, articles of clothing, air samples, liquids or other forensic type samples. Touch DNA and low-template DNA techniques can be further extended by concentrating large sample volumes into volumes more closely matching the analysis volume.

These types of sampling and analysis are advantageously performed for the fields of homeland security, corporate security, and military force protection. Additional fields of use include medical research and diagnostics. For example, sample concentration is useful in determining if catheter or other medical devices are contaminated with bacteria. These devices routinely become contaminated in the hospital setting. However it is often difficult to determine which device is causing an infection. Concentration of wash fluid from these devices allows for rapid detection of the infecting organism. Sample concentration is useful in cancer research where very low concentrations of experimental drugs in body fluids or urine are the targets of analysis, and in allergy diagnosis where low quantities of specific antigens are the targets of analysis in body fluids. Health effects research may also benefit by determining health effects known to be caused by various materials in inhaled particulate matter with aerodynamic diameter below 2.5 microns (PM 2.5). Benefit is seen in the field of forensic medicine where low concentrations of DNA, toxins, or venoms are the targets of analysis in body fluids. Other aspects of use may include the study of operating rooms for surface extraction and air monitoring of pathogens, as well as pharmaceutical manufacturing where the biological aerosol particulate matter concentration is regulated by the United States Food and Drug Administration.

Further, sample manipulation and concentration of targets from complex clinical samples, such as urine, blood, serum, plasma, sputum, and cerebrospinal fluid, for instance, can be used to enable improved detection of pathogens, bacteria, viruses, prions, funguses, extracellular vesicles, exosomes, cancer cells, protein markers, toxins, nucleic acids, and other particles and large molecules of importance to clinical diagnostics. Concentration of target particles and large molecules from these complex samples can benefit from sample addition, membrane blocking, wash steps, and other sample manipulations. More specifically, changes to the sample matrix through sample addition, including pH changes, buffer additions, ionic concentration changes, chelator additions, and addition of surfactants, detergents, and dispersants, along with a host of other sample addition buffers can be used to modify the sample characteristics. Namely these changes can be initiated to modify sample matrix and target particle characteristics, including facilitating changes to particle zeta potential, to inhibit particle aggregation, disaggregate particles, facilitate particle aggregation, and to either increase or decrease capture of small particles to the membrane surface, and to increase or decrease adhesion of all particles or certain classes of particles within the sample matrix to the membrane filter or other surfaces. Additionally, these sample additions may be used to facilitate changes to the membrane filter streaming potential or hydrophilicity and to provide steric hindrance to particle aggregation and particle adhesion.

The described sample addition buffers may be added in small volumes such that the sample volume is only marginally increased or in large volumes so that significant dilution of the sample occurs. Using the sample addition process, a volume of sample addition fluid from 0.5 mL to 1 L, or more preferably from 1 mL to 500 mL of sample addition fluid, may be added to the sample to provide surfactant, detergent, chelator, dispersant, buffer or other additive concentrations that are beneficial or to enable modification of the sample pH.

The most common sample addition buffers that may be used to afford the desired changes to particles and molecules in the sample matrix include, but are not limited to, high ionic concentration to increase particle size through aggregation, low ionic concentration to enable solubilization and disaggregation or to inhibit aggregation, chelators to enable solubilization and disaggregation, depending on materials found in the sample matrix pH may be adjusted up or down to enable solubilization and disaggregation or to enable aggregation, finally surfactants, detergents, and dispersants can be added to improve the hydrophilicity surfaces and to provide steric hinderance to aggregation and to hinder capture by the membrane filter surface and other surfaces. A range of other sample additions may be utilized as will be well understood in the art.

Sample additions may also be utilized to modify the membrane surface and other surfaces that the sample contacts including the sample tube and the concentrating pipette tip outer wall. Sample ionic concentration and pH may be changed and addition of chelators, surfactants, detergents, or dispersants may be utilized to increase the hydrophilicity, change the surface charge, provide steric hindrance layer, and to alter the membrane filter streaming potential.

Sample addition fluids that may be used to enable these changes include, but are not limited to, salts, buffers, chelators, surfactants, detergents, dispersants, blocking agents, and a range of other sample addition fluids and solids that can be used to achieve the desired results. Salts and buffers include sodium chloride, potassium phosphate, ammonium sulfate, phosphate buffer, phosphate buffered saline, Tris buffer, Tris buffered saline, Tris-HCL, HEPES buffer, Tris EDTA buffer. The solutions may be pH adjusted by base or acid addition as would be commonly used in microbiology buffers and other buffers. Chelators that may be used include natural and synthetic chelators such as EDTA, EGTA, Salicylic acid, TEA, and range of other chelators that will be well known by those skilled in the art. Possible surfactants, detergents, and dispersants include a wide range of cationic, anionic, zwitterionic, and non-ionic detergents, Pluronics or poloxamers, and dispersants such as sodium hexametaphosphate, sodium polyphosphate, sodium tripolyphosphate, sodium polyacrylate, polyethylene glycol. Important surfactants include, but are not limited to, Polysorbates such as Tween 20 and Tween 80, Triton X-100, and Chaps. A range of other possible surfactant additions that will improve sample processing and recovery will be well known to those skilled in the art.

Natural soaps and surfactants have been used historically and by indigenous peoples for cleaning wool, garments, and other filter-like materials. Similarly, natural soaps and surfactants can be used to create wet foam, such as from the fruit or roots of the wingleaf soapberry (Sapindus Saponaria), yucca root (Yucca glauca) and other similar natural saponins and related substances, as will be well known to those skilled in the art.

Blocking agents such as Pluronics or poloxamers, polyethylene glycol, and proteins such as bovine serum albumin, non-fat dry milk, polyvinylpyrrolidone, SuperBlock blocking buffer, and a range of other materials, as will be well known by those skilled in the art may be used as sample addition fluids as well.

In the described system blocking steps may be utilized along with the sample addition step or by itself. Blocking steps enable hydrophilic modification, charge modification, streaming potential modification, and addition of steric or sacrificial layers to the membrane filter surface. Membrane blocking is performed by pushing the fluid from the system into the lumen of the hollow fiber membrane filters or onto the membrane filter surface within the concentrating pipette tip. The fluid may be heated or cooled using onboard heaters or chillers and may be allowed to incubate in place for a period of time. Multiple blocking steps can be performed in sequence and the blocking steps can be performed prior to sample processing or at multiple time periods during sample processing or may be initiated based on instrument feedback from onboard pressure transducers or other means used to signal membrane filter fouling.

Blocking agents formulations include, but are not limited to, salts, buffers, chelators, surfactants, detergents, dispersants, blocking agents, and a range of other sample fluids and solids that can be used to achieve the desired results. The primary constituent of these agents are blocking agents, dispersants, or detergents that are able to associate with the membrane surface and modify it to become more hydrophilic, modify the surface charge, or to provide a sacrificial layer that comes off during subsequent steps and allows attached material to also be removed. Use of incubation times, and heated fluid in some cases, tends to improve association of these materials with the membrane surface and thus improves the performance of the blocking step during sample processing. Possible surfactants, detergents, and dispersants include a wide range of cationic, anionic, zwitterionic, and non-ionic detergents, Pluronics or poloxamers, and dispersants such as sodium hexametaphosphate, sodium polyphosphate, sodium tripolyphosphate, sodium polyacrylate, polyethylene glycol. Important surfactants include, but are not limited to, Polysorbates such as Tween 20 and Tween 80, Triton X-100, and Chaps. A range of other possible surfactant additions that will improve sample processing and recovery will be well known to those skilled in the art. Blocking agents such as Pluronics or poloxamers, polyethylene glycol, and proteins such as bovine serum albumin, non-fat dry milk, polyvinylpyrrolidone, SuperBlock blocking buffer, and a range of other materials, as will be well known by those skilled in the art may be used as blocking agents as well.

Blocking fluid may be added to fill the concentrating pipette tip retentate volume fully such that the entire collection surface of the membrane filter is in contact with the blocking fluid for an incubation period. The volume of blocking fluid added is therefore dependent on the concentrating pipette tip design and can have an extremely wide range for use with concentrating pipette tips for clinical diagnostics or DNA concentration, for example, up to large volume concentrating pipette tips for processing of large environmental water samples. Regardless the blocking fluid range will generally range from 0.001 mL to 100 mL or more preferably from 0.05 mL to 50 mL or most preferably from 0.1 mL to 10 mL.

Blocking fluid generally must be incubated to enable modification of the membrane filter surface. Incubation period is a tradeoff due to longer incubation periods providing improved blocking but requiring longer time periods for processing of each sample. Generally, the blocking fluid incubation time period will range from 10 seconds to 30 minutes in length or more preferably from 30 seconds to 15 minutes or most preferably from 1 minute to 5 minutes.

Blocking fluid may be heated to enhance the performance of the blocking step or to reduce the blocking incubation period. The blocking fluid temperature may therefore be heated to a temperature in the range of 30° C. to 99° C. or more, preferably from 35° C. to 70° C., or most preferably from 40° C. to 60° C.

Following the discretionary sample addition and blocking steps the sample processing can be initiated. If membrane filter fouling is low then the sample processing can proceed without interruption, however, if the sample matrix causes fouling then it will often be desirable to implement and filter fouling reduction step. The described systems are capable of applying back pressure to the permeate reservoir and thus forcing fluid to reverse flow through the membrane filter pores and lift material from the surface. This backpulsing may be performed alone or with a push of fluid into the retentate to further move the fouling material from the pores. Backpulsing may be performed by addition of liquid or gas to the permeate reservoir and will most commonly be applied for very short periods of time in the range of 5 milliseconds to 5 seconds or more preferably in the range of 25 milliseconds to 1 second. The pressure is applied at a high frequency throughout the sample processing run or may be applied based on feedback from a permeate flow sensor, permeate pressure transducer, or other means of measuring the sample processing rate or membrane filter pressure drop. One means of measuring the sample processing rate is through a down-facing optical sensor that is used to measure the rate of fluid level drop within the sample container. Using these means the user can set the backpulsing to be performed only after the flowrate drops below a certain set point or after the permeate negative pressure climbs above a certain set point. The backpulsing can then be performed each time that the set point is met or it may be initiated based on the set point and then repeat at the desired frequency until sample processing is complete.

High-frequency backpulsing may be performed by applying positive pressure to the concentrating pipette tip permeate chamber for a time period ranging from 0.1 millisecond to 2 seconds, or more preferably from 1 millisecond to 1 second, or most preferably from 25 milliseconds to 750 milliseconds. High-frequency backpulsing is performed by injecting gas or liquid into the permeate chamber for the described time periods at a frequency of one injection every 0.1 to 10 seconds, or more preferably every 0.5 to 5 seconds, or most preferably every 1 to 3 seconds.

Reverse tangential flow is performed by injecting a wash fluid into the top of the retentate chamber within the concentrating pipette. This injection is performed by injecting enough wash fluid to cause a temporary reversal of flow within the retentate chamber. Reverse tangential flow is performed for a time period ranging from 0.1 millisecond to 1 second, or more preferably from 0.5 millisecond to 750 milliseconds, or most preferably from 1 millisecond to 500 milliseconds. Further, reverse tangential flow is performed at a frequency of one injection every 0.1 to 10 seconds, or more preferably every 0.5 to 5 seconds, or most preferably every 1 to 3 seconds.

Backpulsing may also be used along with a tangential elution flush for improving elution recovery from the concentrating pipette. In this case a volume of gas or liquid, sufficient to create reverse flow through the membrane wall, is injected into the concentrating pipette permeate chamber. The backpulse is performed for a time period ranging from 0.1 millisecond to 1 second, or more preferably from 1 millisecond to 750 milliseconds, or most preferably from 5 milliseconds to 500 milliseconds immediately before or during the tangential elution flush. The tangential elution is performed by injecting the elution fluid into the retentate for a time period ranging from 0.1 millisecond to 1 second, or more preferably from 1 millisecond to 750 milliseconds, or most preferably from 5 milliseconds to 500 milliseconds. This injection may be a continuous flow of fluid or foam or may be made up of shorter pulses of fluid or foam.

For the following description, it can be assumed that most correspondingly labeled structures across the figures (e.g., 132 and 232, etc.) possess the same characteristics and are subject to the same structure and function. If there is a difference between correspondingly labeled elements that is not pointed out, and this difference results in a non-corresponding structure or function of an element for a particular embodiment, then that conflicting description given for that particular embodiment shall govern.

In the following figures, there will be shown and described multiple configurations of disposable concentrating pipette tips which may be used to concentrate biological particles into a reduced liquid volume.

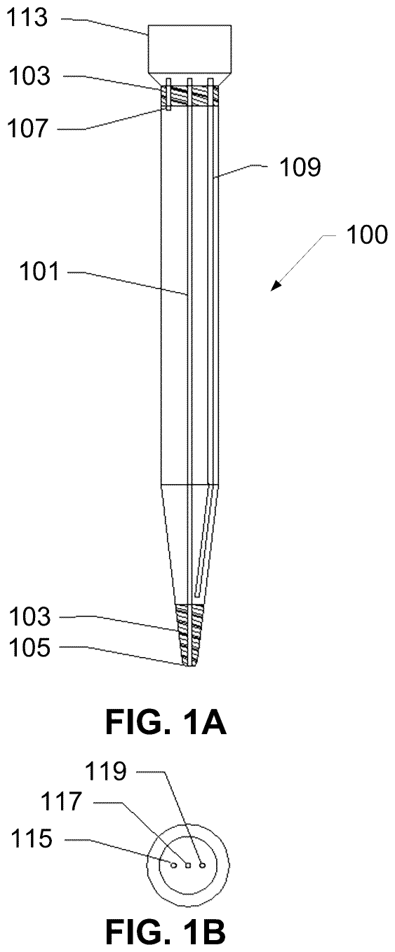

FIGS. 1A and 1B show a concentrating pipette tip (CPT) 100, according to an exemplary embodiment of the present subject disclosure. FIG. 1A shows a CPT 100 includes an opening 105, a hollow fiber filter 101, a permeate purge 107, and a permeate draw 109. CPT 100, including opening 105, fiber filter 101, permeate purge 107, and permeate draw 109 is replaced between samples, removing the potential for cross contamination within the system. Because the sample is aspirated, concentrated, and dispensed with a single instrument, work flow in the laboratory is improved and the required operator skill level is significantly reduced. Automation of the system through platforms similar to those used in automated pipetting workstations will provide a low-cost alternative to automated centrifuge systems with significantly improved operating and higher efficiencies. Multi-tip concentration systems, such as the present subject disclosure, may push the speed of these automated systems an order of magnitude higher.

CPT 100 is a disposable tip that may be constructed by plastic molding techniques. CPT 100 may be, for instance, similar in dimensions to an Eppendorf epT.I.Ps 10 mL tip. CPT 100 includes a connecting portion 113 and an opening 105. Connecting portion 113 allows CPT 100 to be connected to a concentrating unit for operation of CPT 100. Within connecting portion 113, three ports are contained. FIG. 1B shows the three ports, which include a first port 115 connected to permeate purge 107, a second port 117 connected to fiber filter 101, and a third port 119 connected to permeate draw 109. When connected to the concentrator unit second port 117 is in fluidic connection with an elution fluid line originating in the concentrator unit. First port 115 is in fluidic connection with a valve contained within the concentrator unit. Third port 119 is in fluidic connection with a pump contained within the concentrator unit. Opening 105 allows CPT 100 to aspirate a sample into fiber filter 101. Opening 105 provides a small pointed end with a single opening into the lumen of fiber filter 101. CPT 100 also includes potting 103 to secure fiber filter 101, permeate purge 107, and permeate draw 109.

In this configuration, fiber filter 101 is a single hollow fiber filter 101 allowing air to pass through (e.g. microfilter) and is secured into CPT 100 on both ends using potting 103 such that the lumen of fiber filter 101 creates opening 105. Fiber filter 101 may be, for instance, a Spectrum Laboratories, Inc. 100 kD Polysulfone hollow fiber with an internal diameter of 0.5 mm such as those used in the Spectrum Laboratories X1AB-300-04N Module. Connecting portion 113 of fiber filter 101 along with a section of tubing for permeate purge 107 and a section of tubing for permeate draw 109 are all sealed near connecting portion 113 of CPT 100 with potting material 103. In one aspect, fiber filter 101 is one or more hollow fiber filters contained within CPT 100 with CPT 100 being constructed of an impermeable material. Fiber filter 101 or filters and CPT 100 form a permeate chamber between the impermeable wall of CPT 100 and the hollow fiber wall of fiber filter 101.

Hollow fiber filters, such as fiber filter 101, and other membrane type filters are primarily broken into three groups, these are: microfiltration, ultrafiltration, and nanofiltration. Each of these groups is useful for different types of agents being removed from a sample. Nanofiltration filters are not of significant importance here and will not be discussed. Microfiltration refers to those filters with pore sizes of 0.1 micrometer or greater. Ultrafiltration refers to those filters with pore sizes of less than 0.1 micrometer and those in which the pore sizes are generally specified by molecular weight cutoff. Membrane type filters generally are also broken into those specified as hydrophilic and those specified as hydrophobic. In general hydrophobic pore sizes of less than about 0.65 micrometer will not allow aqueous samples to pass through, unless a wetting agent or solvent is used. Hydrophilic filters will readily pass water, but smaller pore sizes, once wet, will not readily allow air to pass until the filter is dried again. In general it is very difficult to dry a wet hydrophilic ultrafilter sufficiently to allow aqueous samples to pass, and additionally, drying ultrafilters can damage the filter resulting in a larger pore size.

Hollow fiber filters made of different materials are used for application specific reasons. Such fibers are commonly made of mixed cellulose esters (ME), polyethersulfone (PES), polysulfone (PS), polypropylene (PP) polyacrylonitrile (PAN), hydrophilic polydivinylidene fluoride (PVDF), and other materials such as stainless steel and ceramics. Various advantages and disadvantages accrue to each type of filter. Some design criteria are the size of pores, biocompatibility, smoothness, fouling potential, and physical strength.

Permeate purge 107 is a tube connecting the permeate chamber formed between CPT 100 and the exterior of fiber filter 101 to a permeate valve within the concentrating unit through first port 115. Permeate purge 107 provides a port for allowing air to flow into the permeate chamber. Allowing air into the permeate chamber is necessary so that liquid that collects in the permeate chamber during processing can be drawn out of the permeate and so that negative pressure in the permeate chamber can be quickly returned to atmospheric pressure. In an alternate embodiment the permeate purge is not in fluidic communication with the permeate valve but is rather a small open port. In this way leakage through the port is small enough to allow the permeate pump to draw sufficient vacuum to allow the sample to be processed, but is large enough so that after the sample is processed the remaining fluid can be drawn out of the permeate due to the inward leakage of air. During elution the permeate pump is also large enough to overcome the permeate purge leakage and increase the pressure in the permeate.

Permeate draw 109 provides a means for drawing the sample through fiber filter 101 and removing the permeate from the permeate chamber formed between concentrating tip 102 and the exterior of fiber filter 101. After permeate flows through fiber filter 101 it is removed using permeate draw 109. Permeate draw 109 extends from near the base inside concentrating tip 100 through third port 119 into a pump within the concentrating unit. Permeate is removed from this location until all of the permeate is removed.

First port 115 for permeate purge 107, second port 117 for fiber filter 101, and third port 119 for permeate draw 109 are each contained within connector 113 on the top end of CPT 100. To operate, CPT 100 is attached to the concentrator unit such that first port 115, second port 117, and third port 119 connect with concentrator unit as described above. A fluid sample is aspirated into opening 105 and through the porous surface of fiber filter 101 using a pump contained within the concentrator unit that is connected to permeate draw 109 through third port 119. In this embodiment fiber filter 101 or other membrane type filter is a dry hydrophilic filter, glycerin filled hydrophilic filter, or other filter type that allows air to pass initially and liquid to pass when contact is made, Thus, air is drawn into opening 105 and through the porous surface of fiber filter 101 until fluid is aspirated into opening 105 and making contact with fiber filter 101 passes through the porous surface.

When the entire sample volume has passed through opening 105, the captured particles on fiber filter 101 are eluted by a tangential flush of fiber filter 101 with a known volume of elution buffer or wet foam. Alternatively a backflush of liquid may be used with a secondary tangential sweep with liquid, foam, or a gas. For a number of reasons the use of wet foam is preferred. Two primary reasons for the preference of foam for elution are (1) that a small volume of liquid may be used to create a large volume of foam, thus allowing for smaller elution volumes, and (2) the created foam is much more viscous than the starting surfactant solution, thus allowing for improved passage of the foam through multiple fiber filters. Immediately prior to tangential elution of the filter the valve controlling permeate purge 107 is opened and the pump connected to permeate draw 109 is allowed to continue running so that any remaining fluid is drawn out of the permeate chamber. After the remaining fluid is drawn out the pump controlling permeate draw 109 is turned off and the valve connected to permeate purge 107 is closed. The permeate chamber may then be left at ambient pressure or pressurized to a positive pressure from 0 to 10 psi above ambient pressure. Removing any fluid remaining in the permeate chamber keeps the fluid from being pushed back into the retentate side of fiber filter 101 and pressurizing the permeate keeps wet foam or the elution fluid from passing through fiber filter 101 into the permeate during elution. As the foam proceeds through fiber filter 101, the foam sweeps the concentrate through CPT 100 and out through opening 105. When the foam has exited CPT 100 it quickly collapses back to a liquid, leaving a final concentrated product of a much reduced volume of liquid. This volume can be in a range of less than 5 microliters to 1 milliliter. In its simplest form, the foam may be made in a separate container, and then injected to sweep the sample from CPT 100 into a sample collection port. However, a sample loop may also be used to measure the amount of liquid used to make the foam. In addition to surfactant foams that are generated by mixing air and a surfactant solution the foam may also be generated with a carbonated surfactant solution. Following carbonation, the solution is agitated by dispensing through an orifice, frit, filter, or capillary tube. The surfactant foam extraction methods described here can also be used for extraction and cleaning of other collection surfaces in aerosol samplers and collectors. The use of foam to extract these surfaces can provide a significant increase in extraction efficiency and significant decrease in final sample volume. In a preferred embodiment the foam is produced by holding a buffered surfactant solution under a head pressure of carbon dioxide and then releasing a volume by opening a timed valve. By controlling both the carbon dioxide pressure and the time that the valve is open the volume of liquid dispensed can be tightly controlled.

For hollow fiber concentration pipette tips using ultrafiltration and microfiltration filters, as may be used for concentration of cellular components, DNA, viruses, bacteria, and other pathogens from a liquid sample, the sample is aspirated simply by drawing a negative pressure on the permeate chamber. In this case air is readily drawn through the fiber filter wall and fluid is aspirated into the lumen of the fiber filter where it then passes through the fiber filter wall.

To further improve the efficiency of the concentration pipette tip, a biocompatible surfactant such as Triton X-100 may be added to the feed at low levels, such as 0.1-0.01% by volume. This liquid is an insignificant volumetric addition, but can increase throughput efficiency from the 40% to 65% range to nearly 100%. Buffered surfactant solutions such as 25 mM tris buffered saline (TBS) or phosphate buffered saline (PBS) with 0.01 to 0.1% Triton X-100 or Tween 20 are commonly used in the collection fluids of bioaerosol samplers.

Mechanical shear such as produced by a shaker motor or ultrasonic horn is also used to improved throughput efficiency and processing speed.

Hollow fiber membrane filters used in the CPT can become blinded due to particle loads in the samples being processed. Methods of reducing blinding are well documented and include tangential flow, high-frequency backpulsing (HFB), vibration, and other mechanisms. Tangential flow is the most commonly used, but it cannot be implemented in its standard form in the CPT. In the CPT system, HFB will be implemented using carbon dioxide from the wet foam elution system to create backpressure on the permeate side of the hollow fibers. The backpressure acts to push captured particles out of the filter pores. The backpressure step is performed in very short pulses with short periods of time between, hence the term high-frequency. In tests of seventy minutes of processing apple juice through single, 0.05 μm hollow-fiber CPT, within approximately 10 minutes after processing began the flow rate had dropped by approximately 50% from 2 mL/min to 1 m L/min. HFB was able to restore the flow rate to the initial flow rate of 2 mL/min and able to maintain a flow rate of greater than 1.3 mL/min throughout the remainder of the 70 minute run. Two short periods of time without HFB cycles resulted in a significant drop in the filter flow rate. The second of these gaps was seen at approximately the 47 minute mark and resulted in a drop in filter flow rate of approximately 50%.

Use of combined HFB and tangential flow is well known in industrial separations and provides the most stable flow rate for those systems by allowing the tangential flow to carry away particles removed by HFB. Because traditional tangential flow cannot be implemented on the CPT a novel oscillating tangential flow (OTF) method may be used. By using a metering pump fluidically connected with the inside of the concentration cell hollow fibers to rapidly move fluid up and down, a tangential flow is set up within the system without removing fluid from the hollow fiber bore. Such a flow over a vertically oriented filter results in significant improvements in filter flow rate with difficult to process samples. Using a metering pump to oscillate the fluid within the CPT rather than oscillating the hollow fibers themselves is seen as more practical implementation of this idea. Implementation of this method is expected to be straightforward and will provide improved sample processing flow rates for difficult to process matrices.

Moreover, using a vertically oriented flat or hollow membrane/filter that extends from the top end of the CPT, i.e., adjacent the connection point to the concentrator, enables particles to be recovered by the tangential flush described herein in a direction of travel from the top to the bottom. Such a tangential flow from the top end to the bottom allows for a very large membrane surface area, and enables processing large volumes quickly, while using only a very small volume of liquid (or wet foam) to be used to recover the particles due to the very small cross sectional area of the retentate. This further allows for greatly increased concentration factors and allows for use in a pipette by the unconcentrated sample being drawn in through the bottom opening and the concentrated sample being dispensed through the same opening. Existing horizontally-oriented systems that do not use the described tangential flushing require that the filtering media be fairly wide to provide a decent processing rate, resulting in smaller elution volumes, i.e. similar to the original sample volume, resulting in very small concentration factors.

Moreover, after processing a sample, the disclosed CPT need not hold the sample volume in the pipette tip. The separate permeate port through the CPT allows the sample volume processed to be governed only by the membrane surface area/membrane flow rate and a time taken to process, versus the limited volume based on the volume of the tip disclosed by the current state of the art.

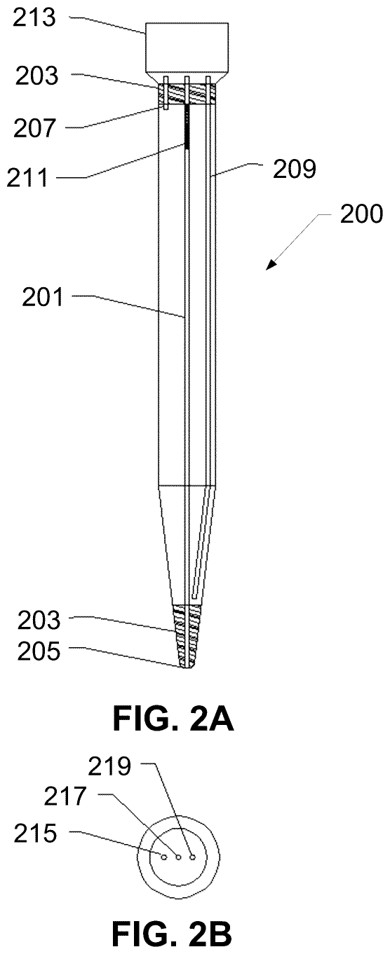

FIGS. 2A and 2B show a similar configuration for a hollow fiber filter 201 that will not allow air to pass through, according to an exemplary embodiment of the present subject disclosure. FIG. 2A shows a CPT 200 including an opening 205, a fiber filter 201, a permeate purge 207, and a permeate draw 209. In this configuration fiber filter 201 has an upper hydrophobic vent portion 211 with the lower portion being hydrophilic 201. Hydrophilic filters will readily pass water, but smaller pore sizes, once wet, will not readily allow air to pass until dried again. The addition of hydrophobic vent portion 211 allows air to pass through the vent until the entire hydrophilic hollow fiber 201 has been filled with liquid sample and

can thus allow it to pass through. In addition to this advantage, use of hydrophobic vent portion 211 allows air to be introduced into CPT 200 after operation is initiated without filling fiber filter 201 with air and thus stopping flow. Hydrophobic vent portion 211 allows the air to pass and liquid to be drawn into fiber filter 201 again. Connecting portion 213 allows CPT 200 to be connected to a concentrating unit for operation of CPT 200. Within connecting portion 213, three ports are contained. FIG. 2B shows the three ports, which include a first port 215 connected to permeate purge 207, a second port 217 connected to fiber filter 201, and a third port 219 connected to permeate draw 209. The remainder of CPT 200 shown in FIG. 2 is identical in configuration to that shown in FIGS. 1A and 1B. To operate, CPT 200 is attached to the concentrator unit and fluid is aspirated into inlet 205 and through the porous surface of fiber filter 201. When the entire sample volume has passed through inlet 205 the captured particles are eluted by a tangential flush of fiber filter 201 with a known volume of elution buffer or wet foam. Alternatively, a backflush of liquid may be used with a secondary tangential sweep with liquid, foam, or a gas.

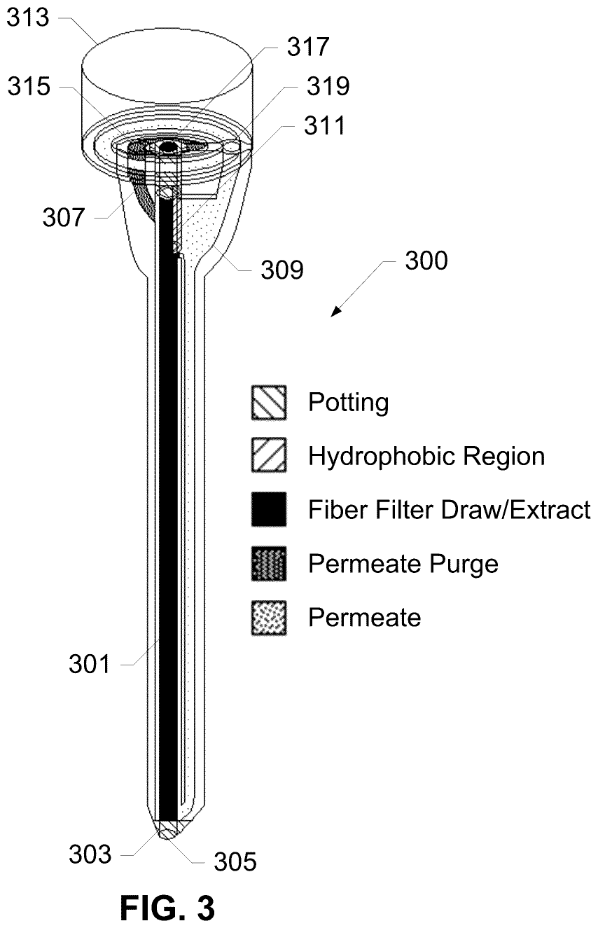

FIG. 3 shows an alternative configuration for connection of a concentrating pipette tip (CPT) 300 to the concentrator unit, according to an exemplary embodiment of the present subject disclosure. In this configuration annular sections within a main female connector 313 mate with the connector on the concentrator unit's male connector. The annular sections of connectors 315, 317, and 319 allow fluid flow between connectors despite the orientation. The primary advantage of the annular connectors is that CPT 300 does not have to be oriented in a specific way, and may spin or otherwise change orientation during use without disruption. In this particular CPT 300 a hydrophobic flat filter section 311 is used for venting.

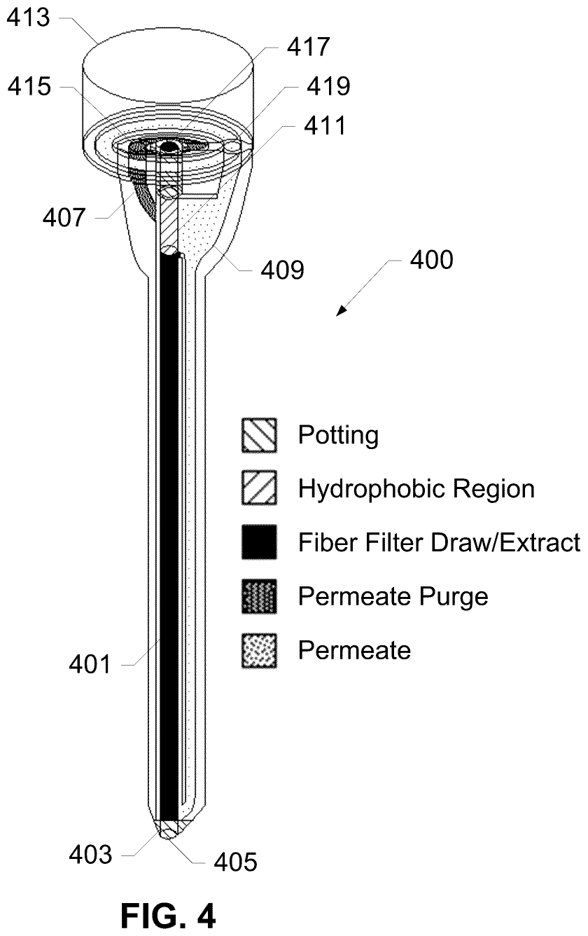

FIG. 4 shows a CPT 400 including an annular configuration for connection to the concentrating unit, according to an exemplary embodiment of the present subject disclosure. In this configuration annular sections within the main female connector 413 mate with the connector on the concentrator unit's male connector. The annular sections of connectors 415, 417, and 419 allow fluid flow between connectors despite the orientation. FIG. 4 shows the same configuration as that shown in FIG. 3 except that a section of the hollow fiber filter 401 is treated to become a hydrophobic vent layer 411 between the hollow fiber lumen and the permeate chamber. Negative pressure applied to the permeate chamber allows air to be drawn through hydrophobic vent filter 411 and fluid is then aspirated in the fiber lumen of fiber filter 401. When the fluid contacts hydrophobic vent filter 411, flow immediately stops. Hydrophobic vent filter 411 may be a flat filter at the top of hollow fiber 401 between the fiber lumen and the permeate chamber or a hollow fiber filter with an upper hydrophobic section of approximately one inch or less with the remainder of the fiber being hydrophilic in nature.

For concentration tips in which air will not draw through the filter, such as ultrafiltration membrane filters that must be packaged wet, methods of contacting sample fluid with the fiber lumen, while not allowing the fluid to exit the disposable tip and contact the concentrator unit, are disclosed. The first method uses a section of hydrophobic vent filter as discussed in FIG. 2 and FIG. 4.

Another method for contacting fluid with the hollow fiber is by using a syringe pump connected to the fiber lumen to draw a volume of air into the syringe body equivalent to the internal volume of the fiber lumen thereby aspirating liquid into the fiber lumen of the fiber filter. In this way fluid does not pass above the disposable tip, but stops at or near the top of the hollow fiber filter.

Another method for contacting fluid with the hollow fiber filter is by using a pump to draw a volume of air out of the fiber lumen and using an optical or other sensor to stop the fluid flow at the top of the hollow fiber filter. An optical sensor can be attached to the concentrator device, rather than to the disposable tip, and monitor a clear section of the disposable tip above the hollow fiber filter. In this way fluid does not pass above the disposable tip.

Another method of contacting fluid with the hollow fiber filter is by dispensing a volume of clean dilution fluid from the concentrator device into the hollow fiber filter and out of the opening and into the sample container. In this way the entire retentate side of the hollow fiber is filled with fluid and the permeate pump can now be activated to draw the sample into the CPT.

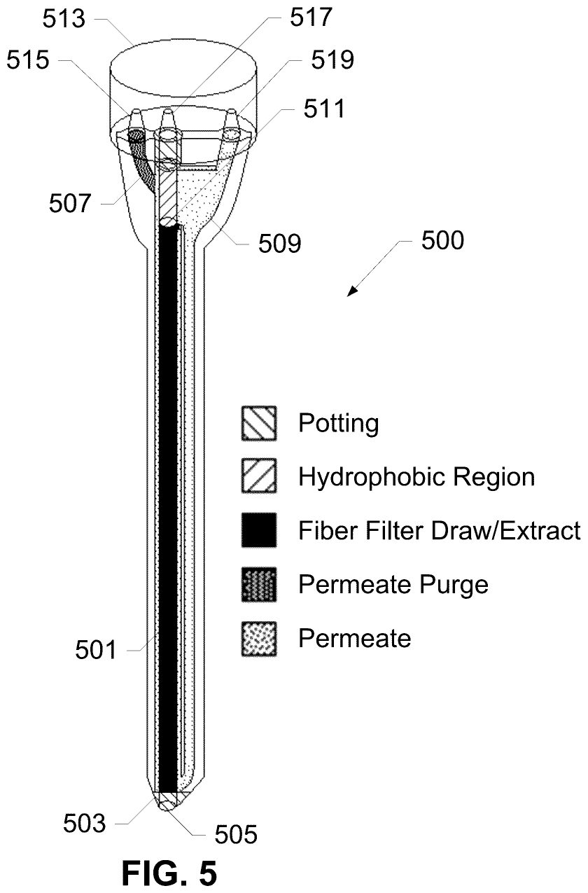

FIG. 5 shows a CPT 500 having pin type connectors 515, 517, and 519, according to an exemplary embodiment of the present subject disclosure. CPT 500 also includes a connector 513, a permeate purge 507, a permeate draw 509, and a hollow fiber filter 501. The CPT in FIG. 5 has a configuration like that shown in FIG. 3, except that the fluidics connections are through three pin type connectors as opposed to the annular connections. Though these connections require a specific orientation, they are more reliable and cost-efficient than the annular connections of FIG. 3.

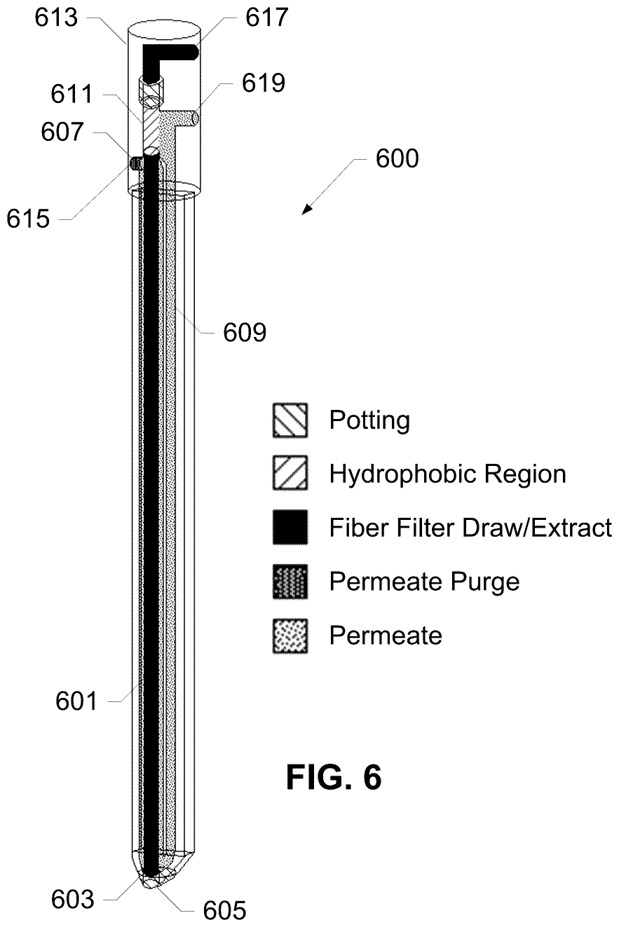

FIG. 6 shows a CPT 600 including a primary male connector 613, according to an exemplary embodiment of the present subject disclosure. CPT 600 also includes a hollow fiber filter 601, a permeate purge 607, and a permeate draw 609. Connector 613 includes fluidics connections 615, 617, and 619 at various lengths from the top end. This tip connects to a female connector with integrated annular connections on the concentrator unit. Hollow fiber filter 601 includes a hydrophobic vent filter 611 near the top.

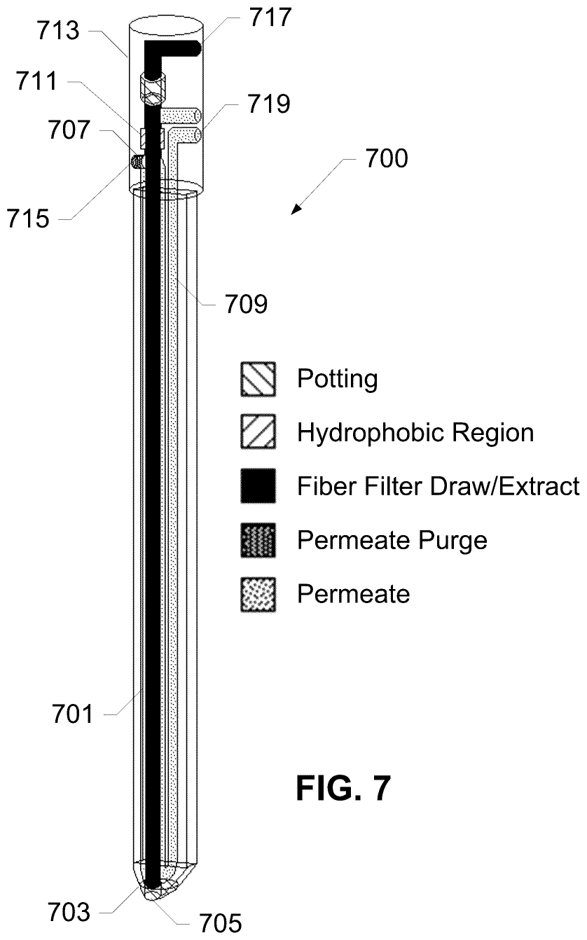

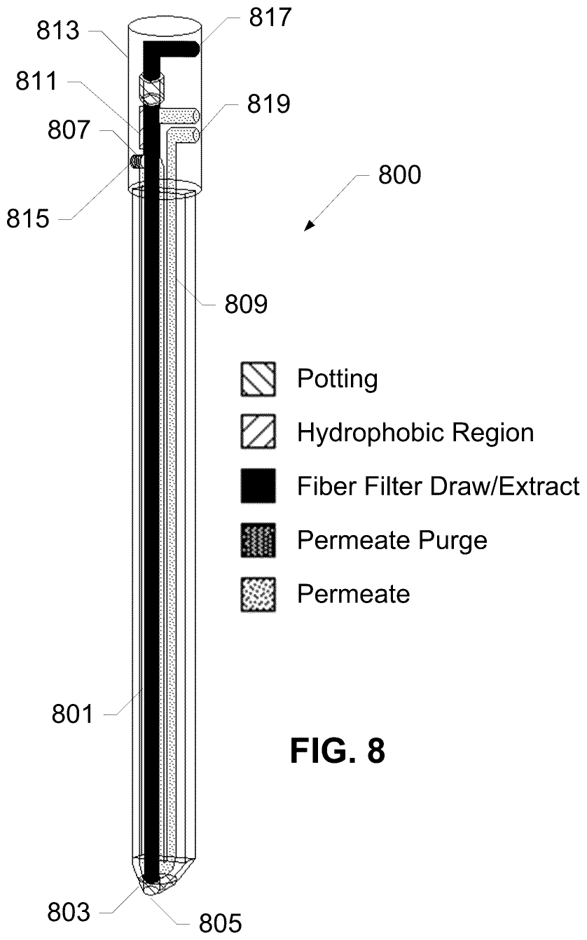

FIG. 7 shows a CPT 700 including a primary male connector 713, according to an exemplary embodiment of the present subject disclosure. CPT 700 also includes a hollow fiber filter 701, a permeate purge 707, and a permeate draw 709. Connector 713 includes fluidics connections 715, 717, and 719 at various lengths from the top end. CPT 700 connects to a female connector with integrated annular connections on the concentrator unit. Hollow fiber filter 701 is similar to the hollow fiber filter of FIG. 6, with the exception that the hydrophobic vent filter is replaced with an integrated conductive sensor 711 to assist in startup.

FIG. 8 shows a CPT 800 including a primary male connector 813, according to an exemplary embodiment of the present subject disclosure. CPT 800 also includes a hollow fiber filter 801, a permeate purge 807, and a permeate draw 809. Connector 813 includes fluidics connections 815, 817, and 819 at various lengths from the top end. CPT 800 connects to a female connector with integrated annular connections on the concentrator unit. Hollow fiber filter 801 is similar to the configuration shown in FIG. 7, with the exception that the conductive sensor is replaced with an optical sensor section that allows for an optical fluid sensor 811 within the concentrator unit to sense the fluid location.

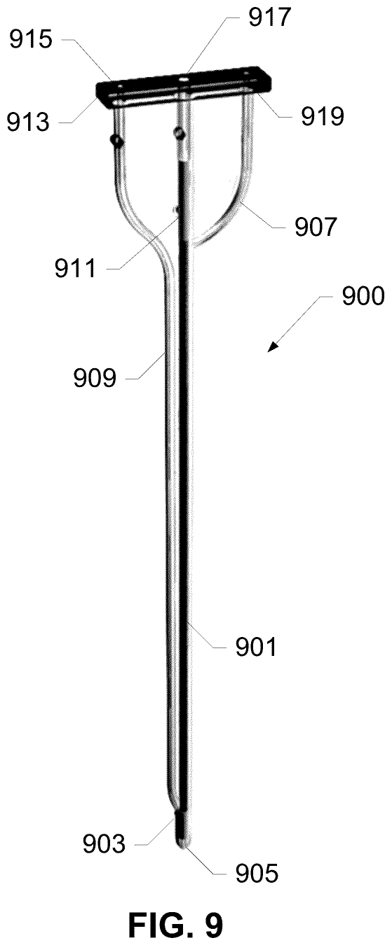

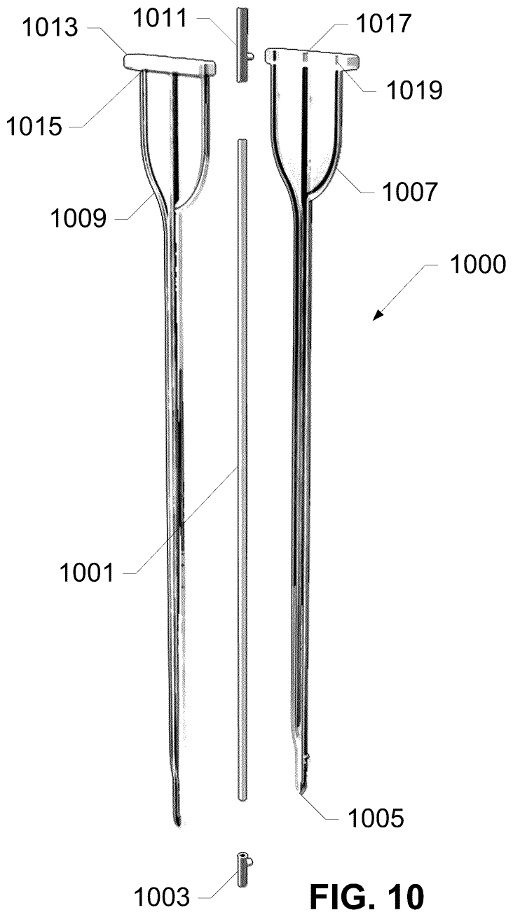

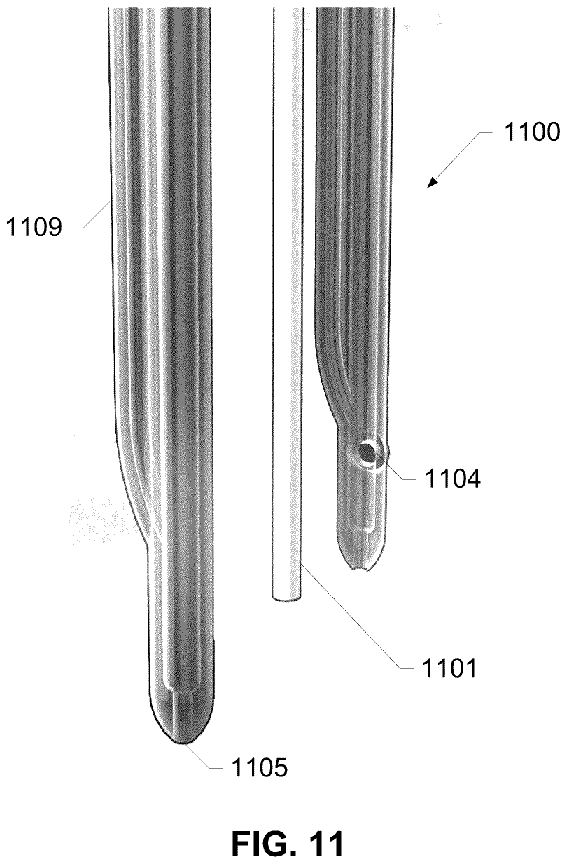

FIGS. 9-11 show one configuration for a CPT, according to an exemplary embodiment of the present subject disclosure. FIG. 9 shows a complete CPT 900. FIG. 10 shows an exploded view of CPT 1000. FIG. 11 shows the port used for potting the lower end of the fiber during production.

FIG. 9 shows a complete CPT 900, according to an exemplary embodiment of the present subject disclosure. CPT 900 includes a connector 913, a hollow fiber filter 901, a permeate purge 907, and a permeate draw 909. Connector 913 includes fluidics connections 915, 917, and 919.

FIG. 10 shows an exploded view of a CPT 1000, according to an exemplary embodiment of the present subject disclosure. Two halves join to make a connector 1013, a permeate purge 1007, a permeate draw 1009, a throughbore for a hollow fiber filter 1001, a hydrophobic vent 1011, and potting 1003. CPT 1000 is snapped together using fasteners. There are many other ways of connecting the two halves that will become apparent to those having skill in the art upon reading this disclosure.

FIG. 11 shows a potting port 1104 fora CPT 1100, according to an exemplary embodiment of the present subject disclosure. Once assembled, potting port 1104 allows the user to put potting into the tip of CPT 1100 where it holds hollow fiber filter 1101 in place. Potting is injected with a syringe or other utensil capable of inserting potting into potting port 1104. A machine assembling the concentrating pipette tip may also employ a syringe or other utensil to insert the potting.

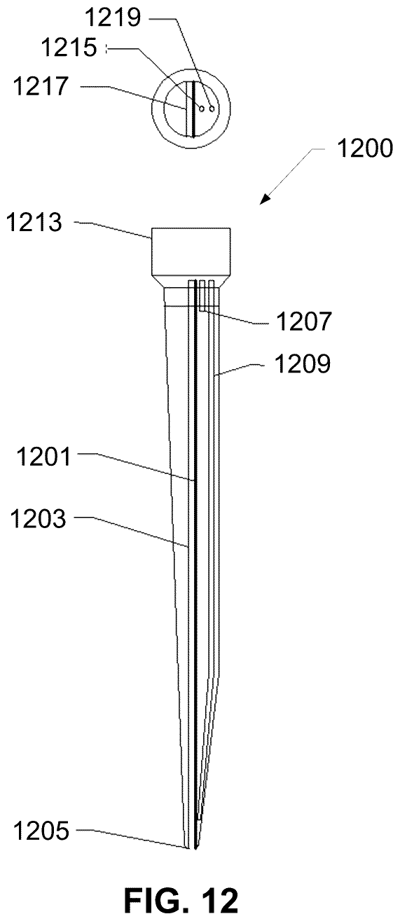

FIG. 12 shows another potential configuration for a CPT 1200, according to an exemplary embodiment of the present subject disclosure. A configuration for a disposable concentrating tip uses a flat porous surface 1201 to divide the tip longitudinally into a permeate side and a side containing a retentate channel 1203. Retentate channel 1203 is enclosed on one longitudinal side by porous surface 1201 and on three sides by the impermeable walls of the tip. Channel 1203 is open on both ends; forming a bottom opening 1205 of the CPT 1200 and the retentate port 1217 contained within connector 1213. The permeate side contains a tube to contain permeate purge 1207 and tube to contain permeate draw 1209. Openings for permeate purge 1207 and permeate draw 1209 are contained within their respective ports 1215 and 1219 contained within connector 1213. To operate, CPT 1200 is attached to the concentrating unit and fluid is aspirated into CPT 1200 and through porous surface 1201. When the entire sample volume has passed through CPT 1200, the captured particles are eluted by a tangential flush of flat porous surface 1201 with a known volume of elution buffer or wet foam. Alternatively, a backflush of liquid may be used with a secondary tangential sweep with liquid, foam, or a gas.

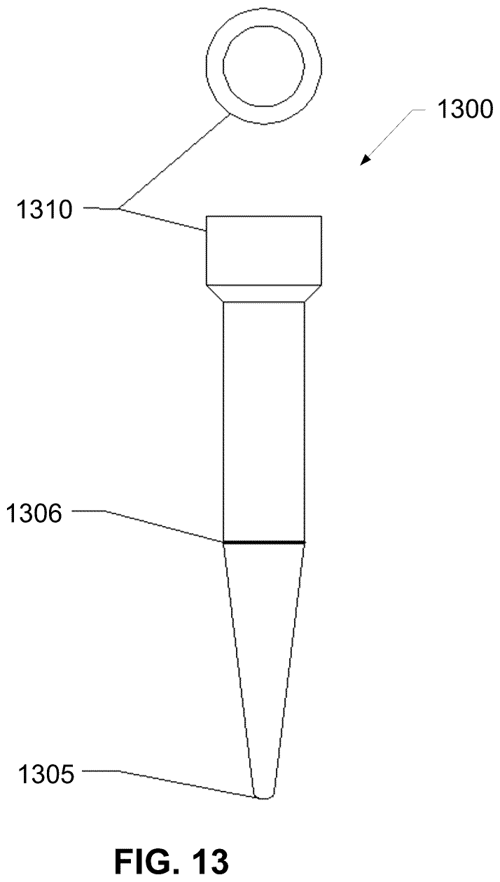

FIG. 13 shows a configuration for a CPT 1300 with a flat porous surface 1306 dividing the tip into an upper portion and a lower portion with an opening 1305 at the lower end and a connector 1310 at the upper end, according to an exemplary embodiment of the present subject disclosure. Porous surface 1306 may be a depth filter, electret filter, microsieve, charged filter, membrane, porous media or other porous surface. To operate, CPT 1300 is attached to the concentrator unit and fluid is aspirated into opening 1305 and through porous surface 1306. When the entire sample volume has passed through opening 1305 then the captured particles are eluted by backflushing the filter with a known volume of wet foam or liquid.

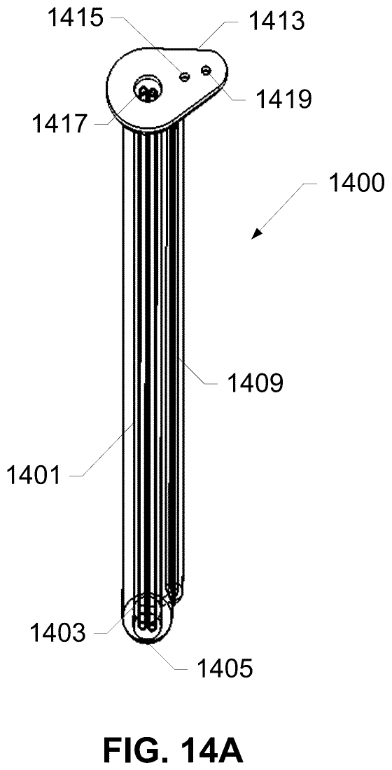

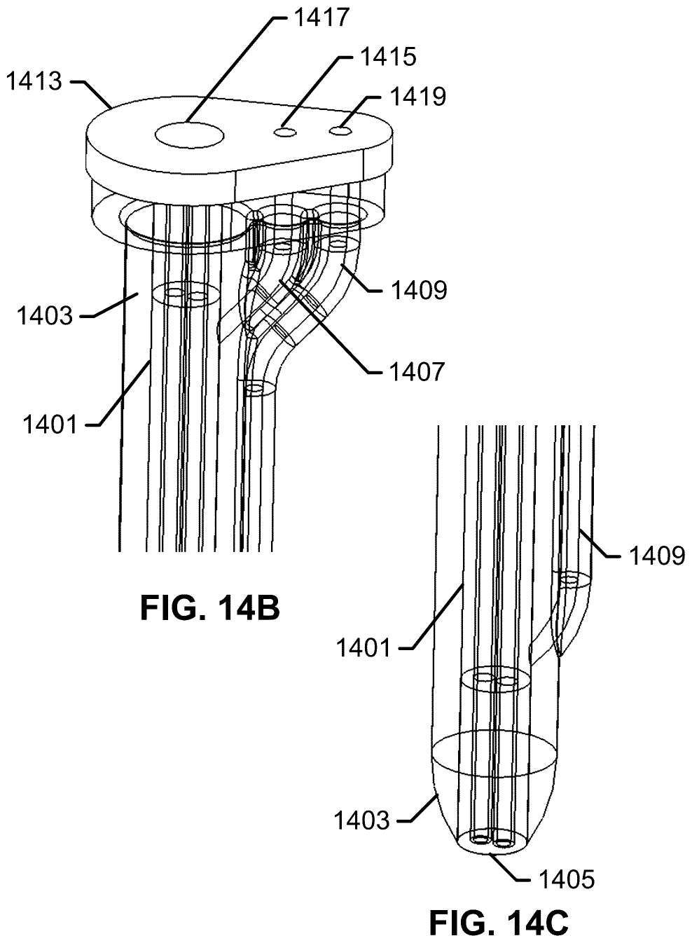

FIG. 14A shows another configuration for a CPT 1400, according to an exemplary embodiment of the present subject disclosure. FIG. 14A shows a CPT 1400 including a connector 1413, two hollow fiber filters 1401, a permeate draw 1409, and potting 1403 to secure the hollow filter. Connector 1413 further includes fluidic connections 1415, 1417, and 1419. Hidden from view underneath connector 1413 is a permeate purge. The permeate purge can be more clearly seen in FIG. 14B.

FIG. 14B shows the end having connector 1413 of the CPT, according to an exemplary embodiment of the present subject disclosure. Connector 1413 includes fluidic connections 1417, 1415, and 1419. Fluidic connection 1415 connects with permeate purge 1407 and a permeate purge line of the concentrating unit. Fluidic connection 1419 ports fluid from permeate draw 1409 to a permeate pump of the concentrating unit. Fluidic connection 1417 ports extraction foam or fluid from the concentrating unit to the hollow fiber filter. Potting 1403 secures hollow fibers 1401 into the CPT.

FIG. 14C shows the end having opening 1405 of the CPT, according to an exemplary embodiment of the present subject disclosure. Opening 1405 receives fluid from a sample for concentration. Hollow fiber filter 1401 is held in place by potting 1403 at opening 1405. Permeate draw 1409 draws permeate from the sample.

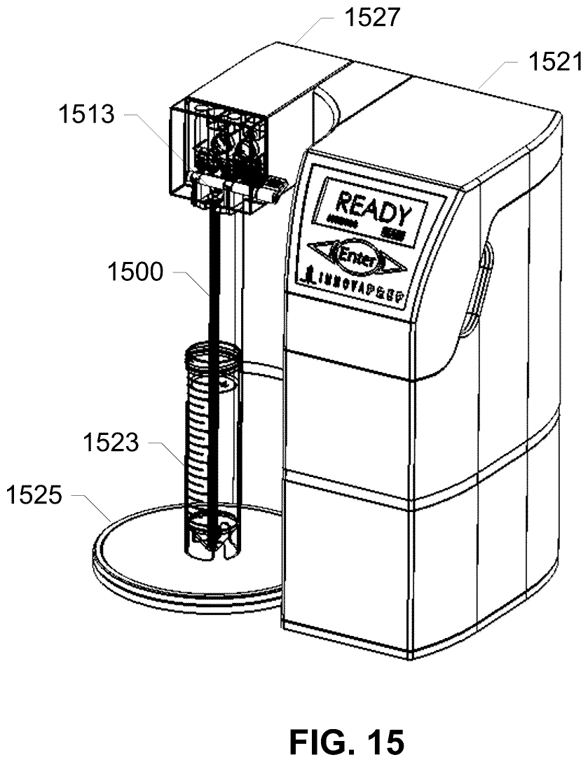

FIG. 15 shows a concentrating unit 1521 gathering a sample 1523 through a CPT 1500, according to an exemplary embodiment of the present subject disclosure. Sample 1523 is placed on a tray 1525 while arm 1527 is raised. CPT 1500 is attached to arm 1527, and arm 1527 is lowered so that CPT 1500 is submerged in sample 1523. An operator then starts concentrating unit 1521, and the sample is aspirated into CPT 1500. When the entire sample has been processed the concentrated sample is dispensed into a sample container.

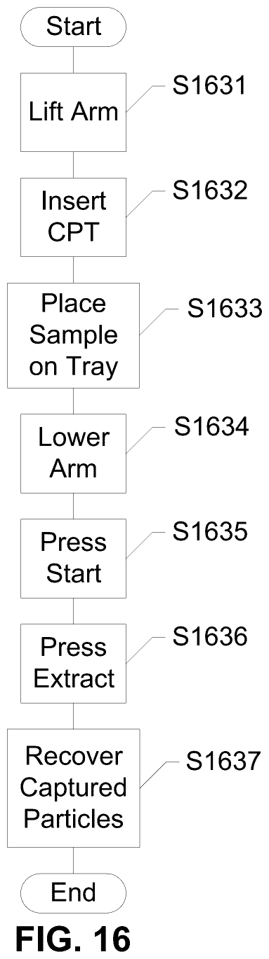

FIG. 16 shows a method of using a concentrating unit having a CPT, according to an exemplary embodiment of the present subject disclosure. First, the arm is raised S1631 so that the CPT can be inserted into the arm S1632. A lever is pushed and the CPT is pushed into the CPT port. The CPT port contains a gasketed sealing surface and a spring loaded surface to hold the CPT ports in place and seal the connections from leakage. This sealing surface contains connectors for the three CPT connecting ports. Next, the sample is placed on the tray S1633. The arm of the concentrating unit is then lowered S1634, dipping the CPT into the bottom of the sample, but without blocking the fiber opening. A user presses start to turn on the vacuum S1635 and the sample begins concentrating within the CPT. Once the sample has been pulled through the CPT, a user can stop the sample processing by pressing a button on the concentrator or the concentrator will detect stoppage of flow through the tip and automatically stop the sample processing. A user may then choose to dispense the concentrate into the original sample container or a user may replace the original sample container with a new extraction sample container. The user then presses the extraction button S1636 activating the extraction cycle. The extraction process is then activated to recover the capture particles S1637 into a concentrated final volume.

In one aspect, the porous surface used for capturing the particles is a flat fibrous type filter, a flat membrane type filter, or a flat porous surface such as a microsieve or nucleopore filter. This flat filter may be positioned lengthwise in the disposable tip such that it separates the interior space of the disposable tip into a retentate side and a permeate side. Capture of the particles of interest and recovery with the elution fluid are performed in much the same way as with the hollow fiber filter disposable tip described above with the exception that capturing and recovery of particles takes place on the retentate side of the flat membrane rather than within the hollow fiber filter lumen. The length of the retentate, in this case, is enclosed on one wall by the porous surface and on the remaining three walls by the impermeable walls of the disposable tip. In the case of the configuration and the hollow fiber filter configuration the particles of interest are recovered by sweeping through the retentate, in a direction tangential to the porous surface, with a foam or liquid elution fluid. Alternatively the particles may be recovered by backflushing the porous surface with a fluid or by any combination of backflushing or tangential flushing with a liquid or gas.

In another configuration the porous surface used for capturing the particles is a filter or porous surface dividing the disposable tip into to a lower retentate reservoir and an upper permeate reservoir. In this case particles of interest are captured onto the bottom side and into the structure of the porous surface. Said particles are then recovered by backflushing the porous surface with a wet foam or liquid elution fluid. The preferred embodiment of this configuration is for charged filters with recovery by way of backflushing with wet foam.

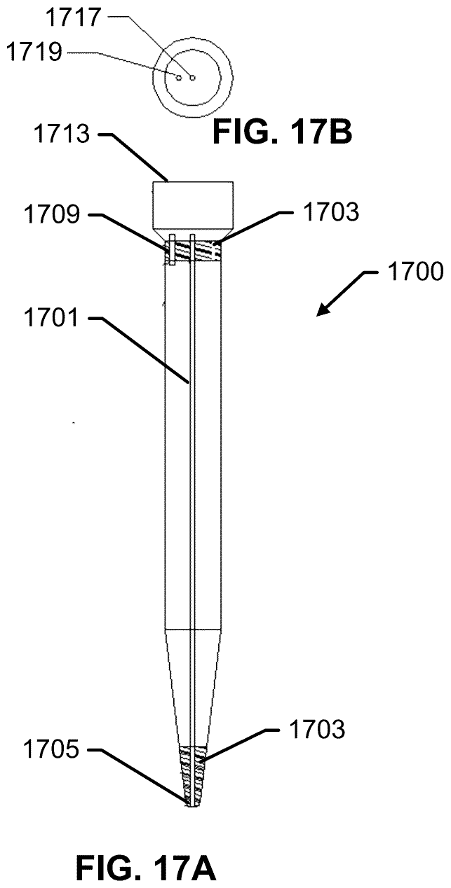

FIGS. 17A and 17B show an alternate configuration for a CPT 1700, according to an exemplary embodiment of the present subject disclosure. FIG. 17A shows a CPT 1700 including an opening 1705, a fiber filter 1701, and a permeate draw 1709. In this embodiment, there is not a permeate purge. According to this embodiment, permeate draw 1709 is shortened, similar to the length of the permeate purge in other embodiments. Each of fiber filter 1701 and permeate draw 1709 is secured within CPT 1700 with potting 1703. A connecting portion 1713 allows CPT 1700 to be connected to a concentrating unit for operation of CPT 1700. Within connecting portion 1713, two ports are contained. FIG. 17B shows the two ports, which include a port 1717 connected to fiber filter 1701 and a port 1719 connected to permeate draw 1709. During operation, the permeate chamber fills with fluid and stays full throughout the sample processing. During elution of fiber filter 1701, instead of pressurizing the permeate chamber a valve is closed on permeate draw 1709 leaving a liquid filled permeate chamber. During elution it isn't necessary to pressurize the permeate chamber because there is void space for the fluid to go into on the permeate side, so the elution fluid or foam will not readily pass through fiber filter 1701.

In one aspect of this configuration, instead of using a permeate valve within the concentration unit a check valve is integrated into the permeate draw 1709 such that a single connection can be used for the CPT. In this way, a sample is aspirated into the CPT and through the filter by applying a permeate pump to connecting portion 1713. The permeate chamber fills will fluid and stays throughout the sample processing. During elution of fiber filter 1701, the elution fluid or foam is dispensed into connecting portion 1713 which causes the check valve with in permeate draw 1709 to close causing the elution fluid or foam to pass through fiber filter 1701.

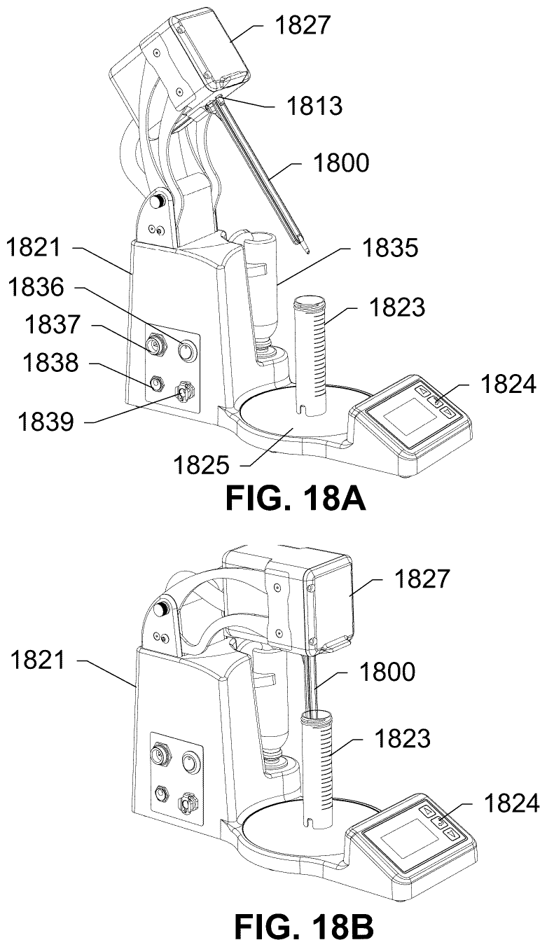

FIGS. 18A and 18B show another concentrating unit for gathering a sample through a CPT, according to an exemplary embodiment of the present subject disclosure. Similar to the unit shown in FIG. 15, the present exemplary embodiment shows a concentrating unit 1821 for gathering a sample 1823 through a CPT 1800. Sample 1823 is placed on a tray 1825, while a fluidics head, or arm 1827 is raised. CPT 1800 is attached to arm 1827 via a CPT interface 1813. In FIG. 18B, arm 1827 is lowered so that CPT 1800 is submerged in sample 1823. An operator then starts concentrating unit 1821 by inputting commands via user interface 1824, and the sample is aspirated into CPT 1800.

When the entire sample has been processed as described herein, the concentrated sample is dispensed into a sample container 1835. Arm 1827 has a quick release fixture that holds CPT 1800 and interfaces with the permeate and elution fluid ports on CPT 1800. Arm 1827 can be raised to allow a sample container to be placed on sample platform 1825, and lowered, to allow CPT 1800 to reach to the bottom of the sample container 1823. A vacuum pump (not shown) is located in the main enclosure of unit 1821. A flexible umbilical cable may be used to connect arm 1827 with the main enclosure with fluid and electrical lines. A permeate outlet port 1839 may be used to dispense the permeate extracted from CPT 1800. A computer interface 1837 is provided to receive commands from and output information to an external computer. A power button 1836 and a power interface 1838 are also provided.

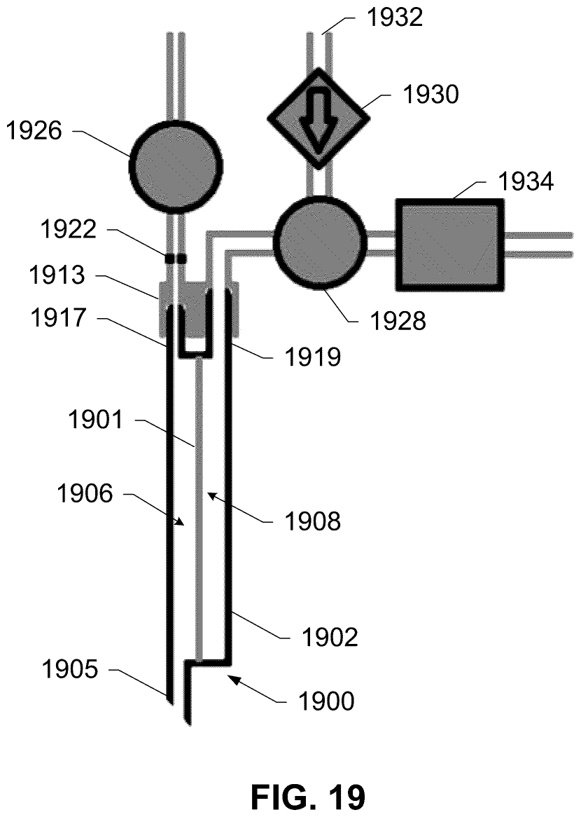

FIG. 19 shows a system for gathering a sample through a CPT 1900, according to an exemplary embodiment of the present subject disclosure. This exemplary embodiment is different from that shown in previous embodiments, in that only two ports are required: an elution fluid port 1917, and a permeate port 1919. However the underlying concept is similar to that outlined in the aforementioned embodiments: a system that utilizes a single use disposable filter cartridge with a sample port that draws in a relatively large liquid sample with a low concentration of particles, the particles are captured on the filter surface while the liquid is drawn through to the permeate, then an elution step resuspends the particles into a relatively low volume of liquid with a high concentration of particles and releases it through the same port the sample was drawn into. The present exemplary embodiment reduces the volume of elution fluid needed, without requiring positive pressure on the permeate side 1908 of filter 1901. Simply, a three-way permeate valve 1928 is closed in order to maintain positive pressure on permeate side 1908, such that any elution fluid stays on the retentate side 1906. Being able to close valve 1928 without using excess pressure enables a more consistent final volume of elution fluid dispensed through port 1905.

In an initial state, elution fluid valve 1926 is closed, three way permeate valve 1928 is linking the permeate port 1919 to vacuum source 1934 with the port leading to the check valve 1930 closed off, and the vacuum source 1934 is deactivated. First, an unused CPT 1900 is connected to the system by inserting the elution fluid port 1917 and permeate port 1919 of CPT 1900 into the CPT interface 1913. The CPT sample port 1905 is lowered into a sample container and therefore the liquid sample therein. At this point an automated concentration process may be initiated, for instance via a user input. Vacuum source 1934 is activated, and the air in the permeate side 1908 of CPT 1900 is evacuated. At this point air can travel through the filter 1901 (which is a hydrophilic filter as described above), therefore the retentate side 1906 of the CPT 1900 is also evacuated of air, resulting in the liquid sample being drawn through sample port 1905 and into the retentate side 1906 of CPT 1900. The liquid passes through the filter 1901, into the permeate side 1908 of the CPT 1900, through the permeate port 1919, through the permeate valve 1928, past vacuum source 1934, and through a permeate outlet. Moreover, the sample fills the retentate side 1906 of the CPT 1900 as high as the exposed area of the filter 1901. The sample does not fill any more of the retentate side 1906 due to the elution fluid valve 1926 being closed, resulting in an air pocket being trapped within the elution fluid port 1917. This prevents the sample from coming into contact with any part of the concentrating unit instrument's fluidics, including orifice 1922 and valve 1926, enabling multiple consecutive uses of disposable CPTs without needing to clean or sterilize the concentrating unit.

As the sample is drawn through the filter 1901, the particles suspended in the liquid sample are trapped on the surface of filter 1901 on the retentate side 1906 of the CPT 1900. Once the entire sample has been drawn through the filter due to vacuum 1934, ambient air continues to enter through the sample port 1905. In the case that a hydrophobic filter 1815 is used, the air will be drawn through the filter 1901 behind the liquid sample into the permeate side 1908. In the case that a hydrophilic filter 1901 is used, the air will not be able to pass through the now wet filter, due to the significant transmembrane pressure required to draw air into the pores of a wetted hydrophilic membrane filter. In this case, the retentate side 1906 of the filter 1901 fills with air, and the filter 1901 will not allow the air to pass through, leaving the permeate side 1908 full or partially full of liquid. The vacuum source 1934 may now be deactivated, and the elution process begins. The permeate valve 1928 switches to link the permeate port 1919 to the ambient air line 1932 through the check valve 1930. This allows air to flow into the permeate side 1908 of CPT 1900, returning it to atmospheric pressure.

An elution foam is used to elute the particles from the filter. An elution fluid is forced into the elution fluid valve 1926 at a high pressure. When the elution valve 1926 opens, the high pressure liquid passes through the orifice 1922. The pressure drop across the orifice controls the flow of the elution fluid and, when using an elution fluid containing a surfactant and carbon dioxide, causes a wet foam to be produced. The wet foam enters the CPT 1900 through the elution port 1917. The wet foam then re-suspends the particles that were captured on the surface of the filter 1901. Meanwhile, the check valve 1930 prevents any flow from the permeate side 1908 of the CPT 1900 to the ambient air port 1932, thereby maintaining a positive pressure in permeate side 1908, and keeping the amount of elution fluid going through the filter 1901 to a minimum. The flow of foam being tangential to filter 1901 enables collection of particles from the retentate side 1906 of filter 1901, resulting in a particle laden foam that exits the sample port 1905 thereby providing a final concentrated sample ready for analysis.

In a three port CPT shown in prior embodiments, the additional permeate line reaching to the very bottom of the permeate side of the CPT enabled all of the fluid in the permeate side of the filter to be removed at the end of the run by allowing air to purge through the top permeate port and sweep the liquid into the line reaching to the bottom of the permeate side. Removing all the liquid from the permeate side is beneficial because it allows gas pressure to be applied to the permeate side, thereby preventing any elution fluid from passing through the filter. This allows for smaller final concentrated volumes, and increases the consistency of the final volume. Applying pressure to the permeate without first removing all of the liquid may cause it to flow back through to the retentate side and thereby increase the retentate fluid volume and retentate fluid volume variability. However, the two port CPT shown in the present exemplary embodiment is less costly to manufacture, and only results in a slight increase in a final concentrated volume, yet being sufficient for its intended purpose.

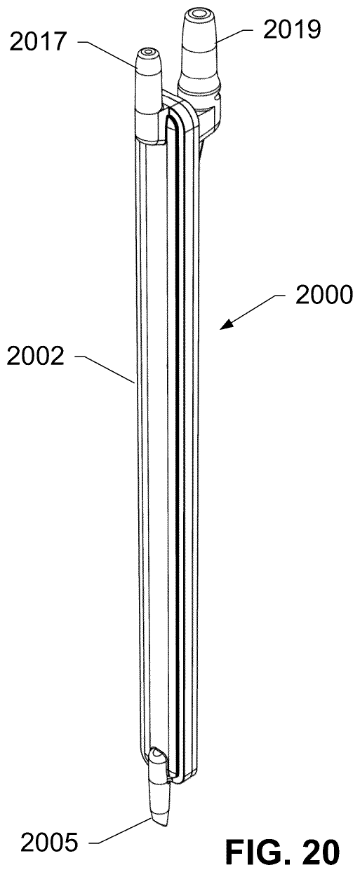

FIG. 20 shows an external view of a CPT 2000 having a flat filter, according to an exemplary embodiment of the present subject disclosure. CPT 2000 comprises a filter housing 2002, an elution fluid port 2017, a permeate port 2019, and a sample port 2005. Although a flat filter is shown, there is no difference in operations of a CPT having a flat filter or any other filter type, as will be disclosed in subsequent embodiments.

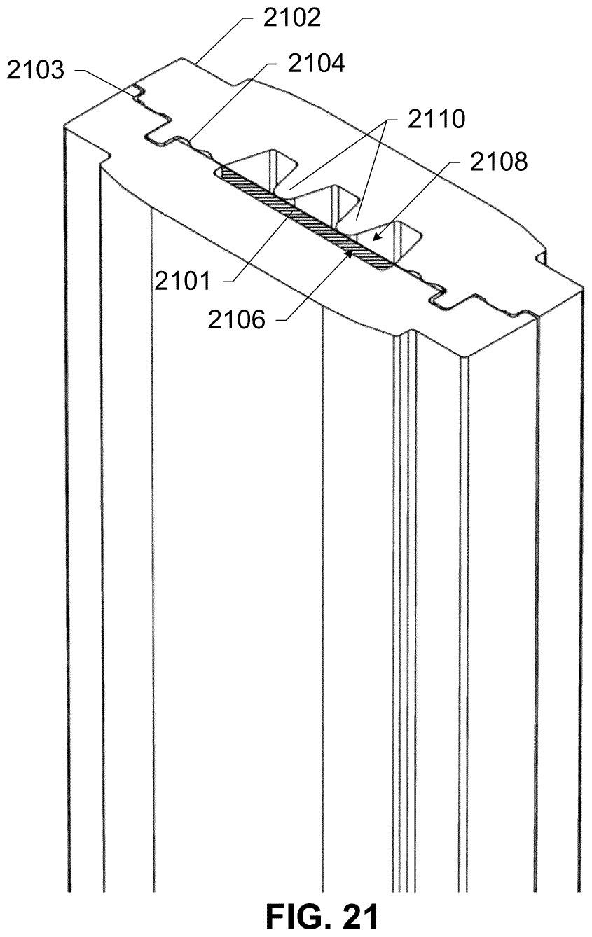

FIG. 21 shows a horizontal cross section of a CPT having a flat filter, according to an exemplary embodiment of the present subject disclosure. Filter housing 2102 comprises a filter housing sealing area 2103, enabling both sides of filter housing 2102 to be coupled together. A filter sealing area 2104 holds in place a filter 2101, which is shown as a flat membrane filter, but can be any filter type. In the case of a flat membrane filter, filter support ribs 2110 enable the filter to stay in the center, and provides space for a retentate side 2106 of filter 2101, and a permeate side 2108 of filter 2101.

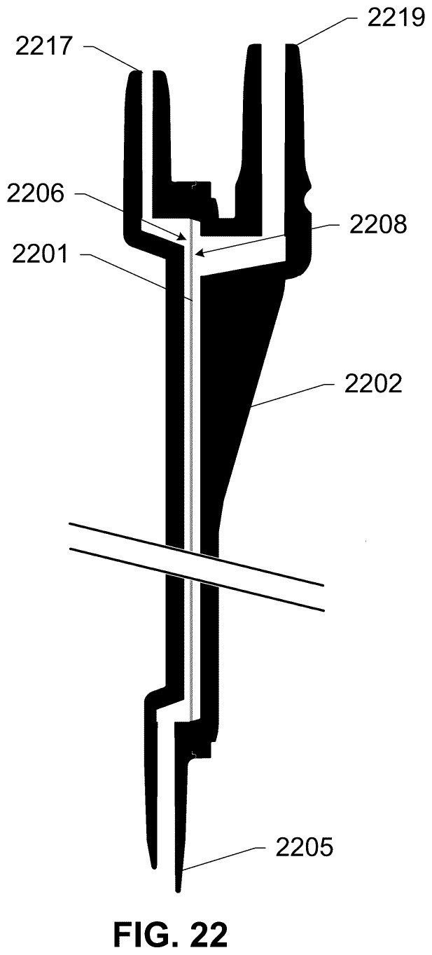

FIG. 22 shows a shortened vertical cross section of a CPT having a flat filter, according to an exemplary embodiment of the present subject disclosure. According to this exemplary embodiment, a CPT comprises a filter housing 2202, an elution fluid port 2217, a permeate port 2219, and houses a flat membrane filter 2201. A retentate side 2206 of the filter is connected to elution fluid port 2217 and sample port 2205, and a permeate side 2208 of filter 2201 is coupled to permeate port 2219.

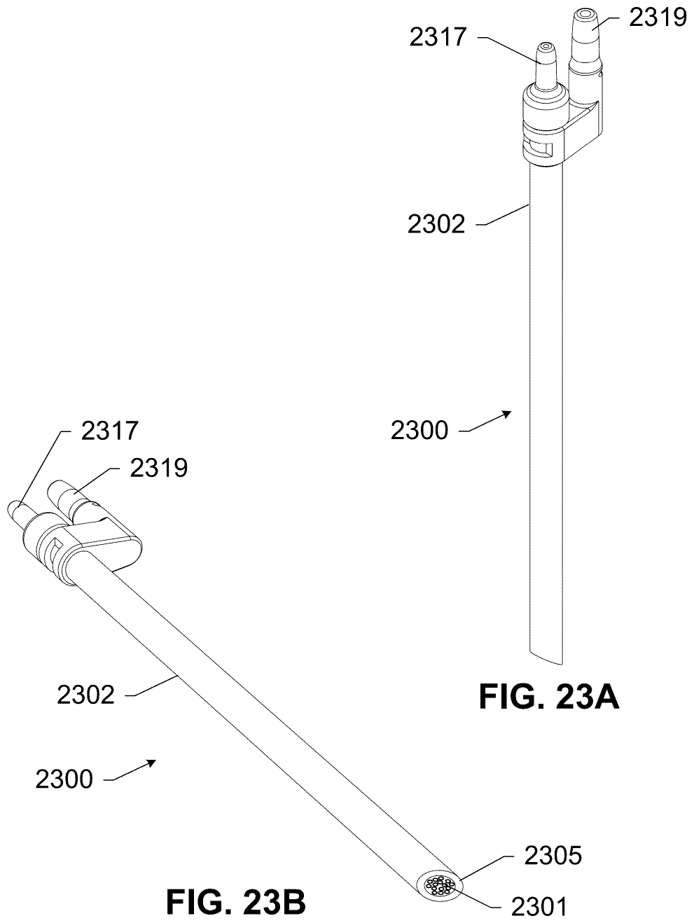

FIGS. 23A and 23B show views of a CPT having a hollow fiber filter, according to an exemplary embodiment of the present subject disclosure. According to this exemplary embodiment, a CPT 2300 comprises a filter housing 2302, an elution fluid port 2317, a permeate port 2319, a sample end 2305, and one or more hollow fiber filter elements 2301 encased in a potting material.

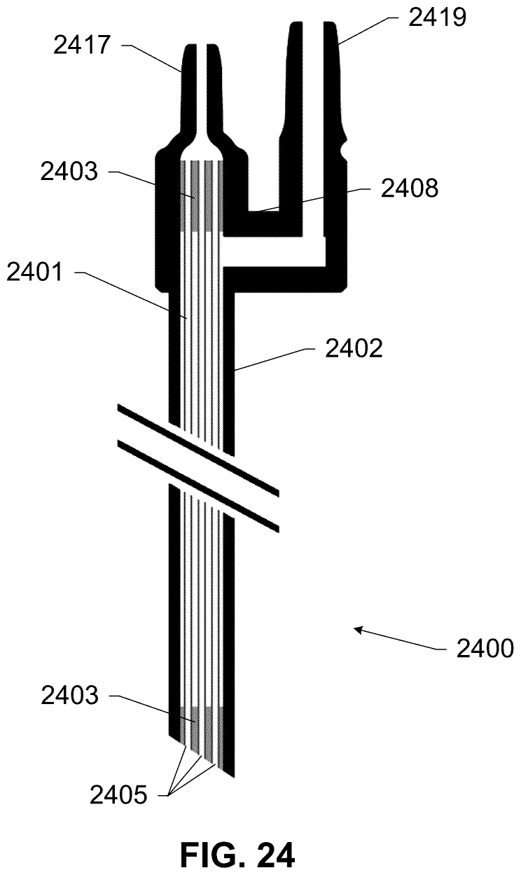

FIG. 24 shows a vertical cross section of a CPT 2400 having a hollow fiber filter, according to an exemplary embodiment of the present subject disclosure. According to this embodiment, a CPT 2400 comprises a filter housing 2402, an elution fluid port 2417, a permeate port 2419, and one or more hollow fiber filter elements 2401, held in place by filter potting material 2403. Although three hollow fiber filter elements 2401 are shown, more or less would be conceivable by persons having ordinary skill in the art in light of this disclosure. Open ends in the hollow fiber filter elements 2401 serve as sample ports 2405 to draw up a sample liquid.

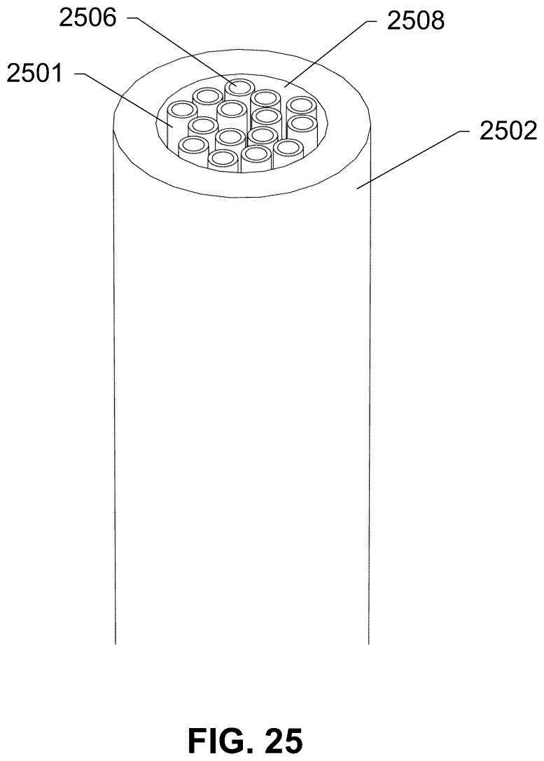

FIG. 25 shows a horizontal cross section of a CPT having a hollow fiber filter, according to an exemplary embodiment of the present subject disclosure. A filter housing 2502 encloses a plurality of hollow fiber filter elements 2501. An inside surface of the hollow fiber filter elements 2501 serves as a filter retentate side 2506, and the outside of the hollow fiber filter elements 2501 serves as a filter permeate side 2508.

The vertically oriented flat and hollow fiber filters in the above embodiments of FIGS. 19-25 extend from the top end of the CPT, i.e., adjacent the elution and permeate ports at the connection point to the concentrator, to the bottom of the filter. As described with respect to FIG. 1, such an orientation and length enables particles to be recovered by the tangential flush described herein in a direction of travel from the top to the bottom, over a very large membrane surface area, and enables processing large volumes quickly, while using only a very small volume of liquid (or wet foam) to be used to recover the particles due to the very small cross sectional area of the retentate. This further allows for greatly increased concentration factors and allows for use in a pipette by the unconcentrated sample being drawn in through the bottom opening and the concentrated sample being dispensed through the same opening. Moreover, the separate permeate port enables the sample volume processed to be governed by the membrane surface area/membrane flow rate and a time taken to process, versus being limited based on the volume of the tip itself, as disclosed by the current state of the art.

Further, as described herein, a volume outside the retentate surface of the filter is in fluid communication with the elution port during elution, and positive pressure on this side during elution may be transferred to the permeate side of the filter during elution. For example, during the filter elution process, the introduction of elution fluid or of wet foam can cause a significant increase in pressure on the retentate side. This increase in pressure is due to the relatively small cross sectional area of the retentate compared to the relatively fast rate at which the elution fluid or foam is pushed through the retentate volume tangential to the filter surface. This momentary increase in pressure can cause a portion of the elution fluid or wet foam to flow through the filter from the retentate side to the permeate side, resulting in reduced elution efficiencies and variable elution volumes.

In order to reduce flow of elution fluid or wet foam from the retentate side to the permeate side, an equal or nearly equal pressure must be applied to the permeate side of the filter. There are multiple ways that this pressure can be applied. After processing a sample, but before elution, negative pressure approaching one atmosphere remains on the permeate side of the filter. In one embodiment, this negative pressure can be relieved using a three-way valve and check valve on the permeate draw, as described herein. During sample processing, the three-way valve is positioned so that flow is allowed through the permeate draw line. After the sample has been processed, but before elution, the three-way valve is actuated so that the permeate draw is closed, but air is allowed to flow through the check valve and into the permeate chamber. The three-way valve is left in this position during the elution process, with the check valve closing off the permeate chamber. In this way, the permeate chamber is maintained at near atmospheric pressure, but is closed off so that very little elution fluid or wet foam can pass through to the permeate side.

In another embodiment, a separate valve may be added to act as a link between the retentate line and the permeate line. After processing a sample the three-way valve and check valve is used to return the permeate chamber to atmospheric pressure. Then the separate valve is opened during the elution process to allow elution fluid or wet foam to momentarily flow towards the permeate chamber (as pressure on the retentate side increases), so that equal, or near equal, pressure is maintained on both sides of the filter.

In another embodiment, pressure may be applied to the permeate chamber using an external pressure source such as a pump, house air, compressed gas, or pressure from the elution fluid container coupled to the concentrating unit. In yet another embodiment, the permeate chamber is allowed to fill, or is intentionally filled, with permeate fluid or another incompressible fluid and is valved closed, so that no space is available for elution fluid or wet foam to travel through to the permeate chamber. In this way the entire elution fluid or wet foam is allowed to act on the retentate side of the filter during the elution process.

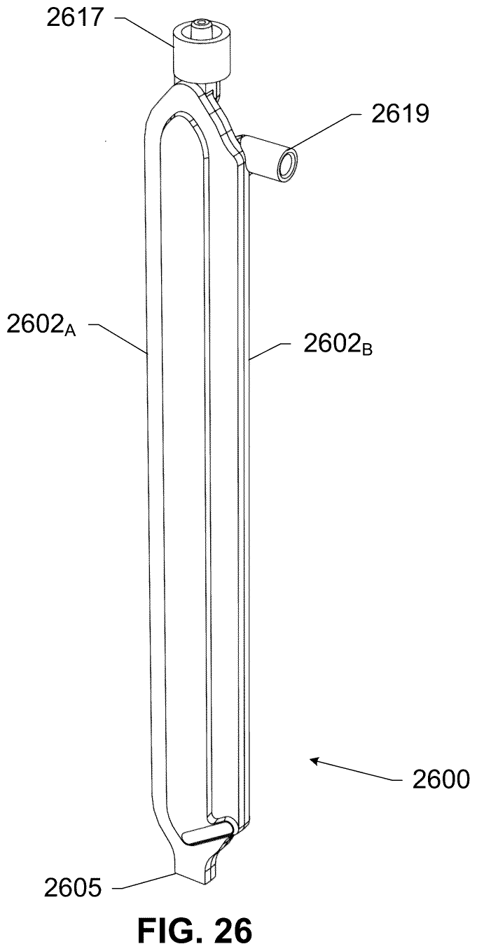

In exemplary embodiments of the present subject disclosure, the concentrating pipette tip (CPT) may include two filters instead of one, thereby increasing the surface area of the filter without increasing the size of the cartridge housing. FIGS. 26-28 describe a CPT having two filters, according to exemplary embodiments of the present subject disclosure. FIG. 26 shows an isometric view of a CPT 2600 having two filters, according to an exemplary embodiment of the present subject disclosure. CPT 2600 is constructed with two housing halves 2602_{A} and 2602_{B}, each of which houses a filter with a permeate side and a retentate side. CPT 2600 further comprises an elution fluid port 2617 enabling foam to be let in to CPT 2600, a permeate fluid port 2619, and a sample end 2605 that enables letting in a sample and providing a channel for the retentate fluid.

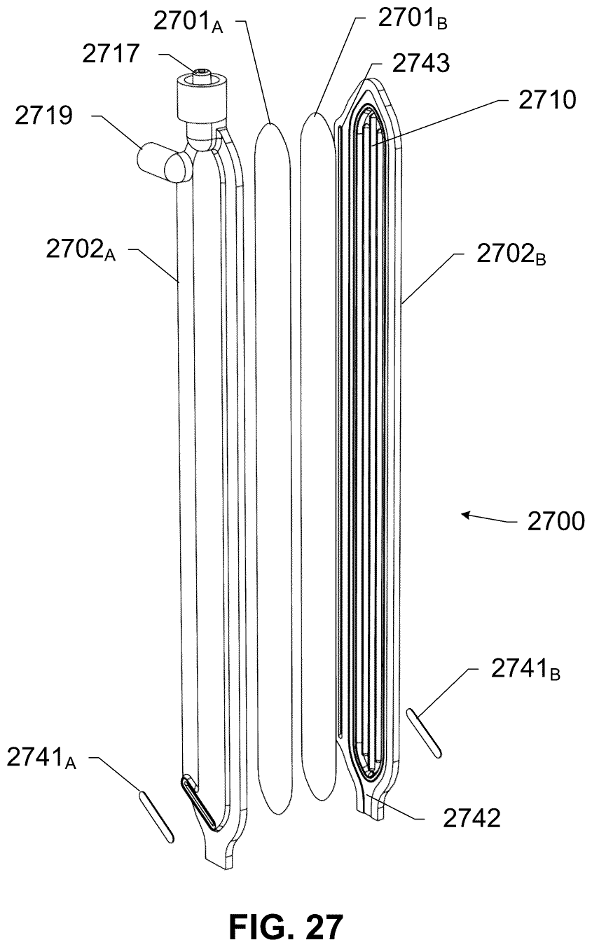

FIG. 27 shows an exploded view of a CPT 2700 having two filters, according to an exemplary embodiment of the present subject disclosure. CPT 2700 includes two housing halves 2702_{A} and 2702_{B} that may be sandwiched together to house flat filter membranes 2701_{A} and 2601_{B}, a permeate fluid channel 2743 that is in fluid communication with permeate port 2719, filter support ribs 2710, and a retentate fluid channel 2742 that is in fluid communication with an elution fluid inlet port 2717. Retentate channel 2742 is formed by the space between the two halves 2702_{A} and 2702_{B} being sealed together. Further, front and rear permeate channel covers 2741_{A} and 2741_{B} are used to cover the permeate channel, and allow for draining the permeate channel.

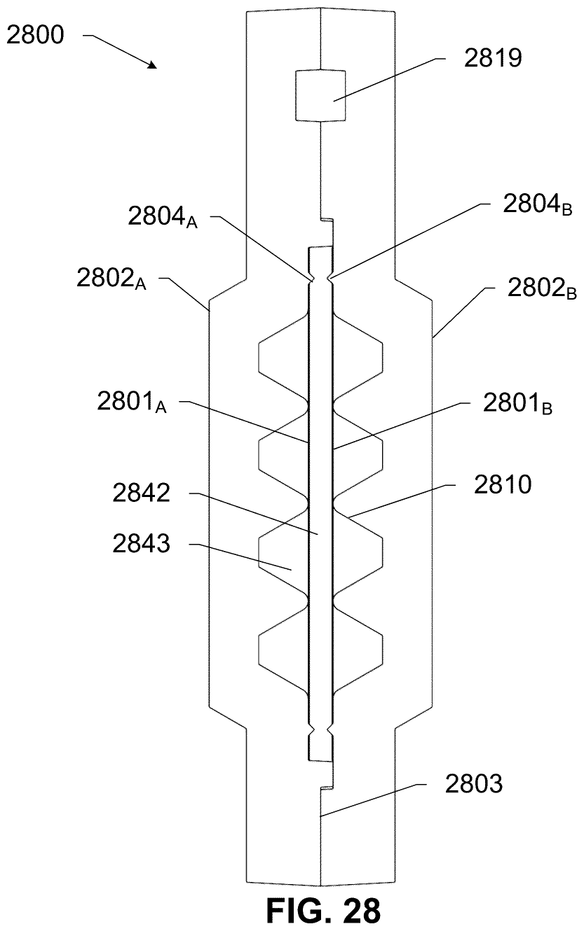

As described above, the presence of two filters provides a larger surface area when compared to the single-filter designs, thereby increasing the sample flow rate, and reducing the effects of particle loading. To achieve a similar surface area with a single filter would require a significantly larger housing, which would in turn reduce elution efficiency. Further, the cross sectional geometry is improved when compared to a single filter design. FIG. 28 shows a cross-sectional view of a CPT 2800 having two filters, according to an exemplary embodiment of the present subject disclosure. Similar to FIGS. 26 and 27, CPT 2800 has two housing halves 2802_{A} and 2802_{B} that may be sandwiched together to house a permeate line 2819 in fluid communication with a permeate draw and a permeate fluid channel 2843, a pair of filters 2801_{A} and 2801_{B} that are coupled to the top of the housing halves by filter sealing areas 2804_{A} and 2804_{B} and held in place by filter support ribs 2810, and a retentate fluid channel 2843 formed by the space in between the two housing halves 2802_{A} and 2802_{B}. Filter housing sealing area 2803 provides a contact point for connecting the two housing halves 2802_{A} and 2802_{B}.

The pair of vertically oriented filters in the above embodiments of FIGS. 26-28 extend from the top end of the CPT, i.e., adjacent the elution and permeate ports at the connection point to the concentrator, to the bottom of the filter, i.e. adjacent the sample draw/retentate fluid port. As described, such an orientation and length enables particles to be recovered by the tangential flush described herein in a direction of travel from the top to the bottom, over a very large membrane surface area, and enables processing large volumes quickly, while using only a very small volume of liquid (or wet foam) to be used to recover the particles due to the very small cross sectional area of the retentate. In addition to the larger surface area gained by the two-filter CPT, the vertical orientation further allows for greatly increased concentration factors and enables efficient usage of disposable or single-use CPTs by drawing in the sample and dispensing the retentate fluid through the same opening.

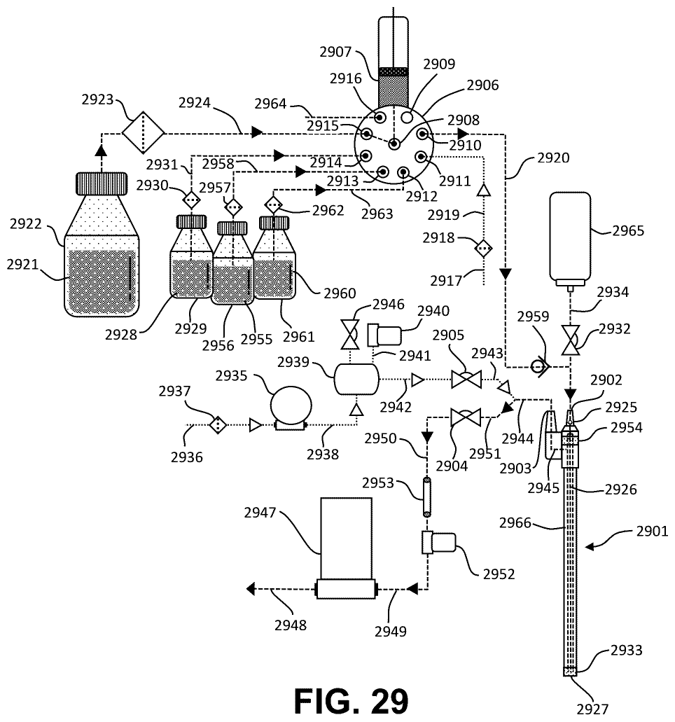

An exemplary embodiment of the present subject disclosure is shown in FIG. 29. This embodiment enables liquid samples to be processed and biological and non-biological particles and large molecules present in the samples can then be manipulated in various ways prior to being eluted in a concentrated sample volume.

The process is especially amenable to automation of complex processes. These processes for particle manipulation include, but are not limited to concentration, wash steps, buffer exchanges, labeling, dying, lysis and a variety of other treatment steps that are routinely performed in the laboratory using automated, semi-automated, and manual treatment methods.

The manipulation or treatment steps can be selected by the user from a menu or custom methods may be used by modifying available instrument methods. After selecting the appropriate steps CPT 2901 is manually or automatically inserted into the instrument with retentate port 2902 and permeate port 2903 inserted into the appropriate ports on the instrument. Following insertion of CPT 2901 into the appropriate ports, the user places the sample container holding the sample to be processed in the appropriate location under CPT 2901 or in the case of an automated system on or more samples will be placed in the appropriate locations. The described embodiment can be configured in an automated robotic manner such that it is capable of processing more than one sample can be automatically processed into a single, pooled and concentrated sample. Additionally, in a semi-automated system multiple samples can be processed with the user alerted to place the samples in the appropriate location at the required time.

The sample process can be initiated by the user by pressing the appropriate user interface controls on the instrument. As stated earlier there are a number of possible manipulation steps that can be performed using the described embodiment. Each possible manipulation process that can be performed will now be described in what is the most common order of processing. Other possible manipulations and modifications to these manipulations, as well as the order that the manipulations are performed with the described embodiment, can be modified as necessary with the described embodiment allowing for significant freedom for the operated to change the processes to achieve desired results. As one of ordinary skill in the art would appreciate, other sequences of steps may be possible.

The specific processes to be described for this exemplary embodiment include: injection of sample additive into the sample, membrane blocking, sample processing, membrane filter fouling treatment, sample washing, sample treatment, sample elution for treatment outside of tip prior to reprocessing in the tip, sample labeling, sample dying, and elution into a concentrated sample. As stated earlier this exemplary embodiment enables manipulation of samples in a number of ways and the list provided is not anticipated to be all encompassing as will be understood by one skilled in the art.

In some applications there may be advantages to adding additives to the sample being processed. These additives may be surfactant solutions, ionic solutions, coagulants, buffer solutions, chelators, higher or low pH solutions meant to change the pH of the sample, a solvent solution, or a range of other solutions that can be used to modify the sample in some way. This list of possible solutions is not anticipated to be all encompassing and many other solution types could be used as will be understood by one skilled in the art. The reasons for performing a sample addition step include, but are not limited to: causing changes to interactions between target viral, bacterial or other particles and non-target particles present in the sample; changing pH or other sample characteristics to modify the solubility of natural organic matter, including humic and fulvic substances, proteins, polysaccharides, and other target and non-target particles and molecules; modify particle zeta potential through fluid ionic strength changes or additives for enhancing steric hinderance; improving recovery of target particles by adding Tween 20, Tween 80, sodium polyphosphate, glycine or other surfactants, proteins, chemical dispersants, or other chemicals that are known to improving recovery of target particles from membrane filters; addition of DNase or RNase to remove free DNA; free RNA or DNA or RNA associated with cellular debris; addition of intercalating dyes such as propidium monoazide or ethidium monoazide to bind free DNA; addition of coagulants or flocculants such as for removing natural organic matter including humic or fulvic acids or other organic material such as proteins, polysaccharides, other carbohydrates, or any other number of materials that may cause in pore fouling. Coagulants and flocculants may be selected from aluminum-based coagulants, ferric-based coagulants, polymeric flocculants, organic polyelectrolytes, and other available coagulants and flocculants that will be well known to those skilled in the art. Many other sample additions that will be known to those skilled in the art may also be added using the described approach.

To inject a sample additive into the sample, permeate valve 2904 and permeate pressure valve 2905 are closed, allowing the permeate 2966 of CPT 2901 to be sealed off from atmosphere. Foam elution valve 2932 is closed and remains closed except when wet foam is being dispensed during other operations. The multiport rotary distribution valve 2906 is used to connect syringe pump 2907 to one of six ports through central port 2908. The distribution valve 2906 is first positioned to connect port 2911 to central port 2908 and syringe pump 2907. Syringe pump 2907 is then activated to draw air from air inlet 2917 through high efficiency particle arresting (HEPA) filter 2918, fluid line 2919, port 2911, central port 2908 and then into the syringe 2907. A volume of air equivalent to or slightly more than the internal volume of fluid line 2920 plus the internal retentate flow path through CPT 2901 is drawn. The air drawn into the syringe will later be used to flush sampled additive out of fluid line 2920 and the internal retentate flow path of CPT 2901.

Next, rotary distribution valve 2906 is positioned so that port 2915 is connected to central port 2908. Syringe pump 2907 is then used to draw sample addition fluid 2921 from solution bottle 2922 through sterile filter 2923, sample addition fluid line 2924, and then into syringe pump 2907. The syringe pump 2907 draw volume is equivalent to the required sample addition volume.

After the sample addition fluid 2921 is drawn into syringe pump 2907, distribution valve 2906 is positioned so that syringe pump 2907 and central port 2908 are connected to port 2910. Syringe pump 2907 is then activated to dispense sample addition fluid 2921 through fluid line 2920, check valve 2959, and into CPT 2901 through CPT retentate port 2902. Sample addition fluid 2921 flows through check valve 2925 and down the lumen of the hollow fiber membrane filters 2926 within CPT 2901 and out through CPT opening 2927 and into the sample. Syringe 2907 is driven until all fluid and air within is expelled. In this way, the air in syringe 2907 is used to push sample addition fluid 2921 fully through fluid line 2920, check valve 2959, and the internal retentate flow path of CPT 2901 and out CPT opening 2927 and into the sample.

The flowrate that syringe 2907 dispenses the sample addition fluid 2921 at can be adjusted based on the number and length of hollow fiber membrane filters 2926 and the pressure drop associated with the fluid line 2920, check valve 2959, and check valve 2925. The flowrate is optimized to ensure that sufficient pressure drop is produced at the top end of the hollow fiber membrane filters 2926, so that relatively equivalent volumes are pushed into each hollow fiber. Further, the flowrate is optimized to reduce the flow of sample addition fluid 2921 through the wall of hollow fiber membrane filters 2926 and into the permeate 2966. For clarity, the retentate of CPT 2901 is made up of the internal flow path of the hollow fiber membrane filters 2926 plus the interior flow path within CPT retentate port 2902 that is fluidically connected to the internal volume of hollow fiber membrane filters 2926. Permeate 2966 is made up of the enclosed area between the outer wall of hollow fiber membrane filters 2926 and the wall of CPT 2901 and enclosed between potting 2933 and 2954. Additionally, slight positive pressure can be applied to the permeate 2966 to reduce the passage of sample addition fluid 2921 into the permeate 2966.

To apply positive pressure, air pump 2935 is first activated and air is drawn in through air inlet 2936 through HEPA filter 2937. The air is pushed through fluid line 2938 and into pressure chamber 2939 until a preset pressure is reached according to pressure transducer 2940 which is fluidically connected to pressure chamber 2939 via fluid line 2941. With air pressure controlled at the preset pressure, permeate pressure valve 2905 is opened to allow pressurized air to flow through fluid line 2942, the permeate pressure valve 2905, fluid line 2943, fluid line 2944 and into CPT 2901 through permeate port 2903. Specifically, the pressurized air flows through CPT 2901 permeate path 2945 and into permeate side 2966. Permeate valve 2905 can then be closed, allowing pressure to remain on the permeate side 2966. Air pressure will normally be applied immediately after the sample addition fluid 2921 has reached CPT opening 2927. In this way the hollow fiber membrane filters 2926 are wetted out and do not readily allow air to pass through into the retentate. The positive pressure applied to the permeate 2966 is generally between 0.1 psi and 50 psi and more specifically between 0.5 psi and 5 psi. Depending on the characteristics of the hollow fiber membrane filters 2926 and sample addition fluid 2921 atmospheric pressure may be applied or slight negative pressure can be applied to the permeate 2966 for a short period of time before positive pressure is applied to ensure that the hollow fiber membrane filters 2926 fully wet out and do not allow the pressurized air to pass.

Atmospheric pressure can be applied by opening pressure vent valve 2946 and then opening permeate pressure valve 2905 to vent the permeate. A HEPA or other vent filter may be used on the open side of vent valve 2946 to reduce the potential for introduction of particles from atmospheric air. Pressure valve 2905 may then be closed if sealing off of the permeate side 2966 is desired. Negative pressure can be applied by closing permeate pressure valve 2905, opening permeate valve 2904, and turning on permeate pump 2947. The pump will then apply negative pressure to the permeate 2966 by pushing air or fluid out through fluid line 2948 while pulling air through fluid lines 2949, 2950, 2951, and 2944 and through permeate valve 2904 and permeate path 2945. The negative pressure on the permeate can be measured and controlled during this process by using pressure transducer 2952 and permeate pump 2947. The flowrate through the permeate can also be measured using permeate flow meter 2953.

A number of similar configurations can be used for applying negative, atmospheric or positive pressure to the permeate 2966 as will be understood by those skilled in the art. These configurations can include the use of different types and styles of check valves or electronically controlled valves to enable connection to a vacuum pump or to a pressure reservoir or atmospheric vent. Further use of pressure transducers and flow meters enable control of these parameters in the system.

One or more sample addition fluids can be added to the sample using the described approach if multiport valves with more ports and additional bottles are connected to the system. The sample addition fluids may also be heated or cooled before addition to the sample by heating or cooling jackets around the sample additional fluid bottle or using a heated or chilled fluid line. Following sample addition, the sample with additive can be subjected to additional treatment steps such as vortexing, stirring with a magnetic stir bar or other stirring mechanism, shaking, heating, cooling, sonication, bead beating or any number of other treatments that will be known to those skilled in the art. Additionally, using a similar approach to that described for sample additive addition, air can be pushed through CPT 2901 and into the sample as a method of mixing the sample. The sample may also be left at room temperature or a heated or cooled temperature for a period of time before proceeding with sample processing.

After the sample additive has been pushed out of CPT 2901, a membrane filter blocking step can be performed. The blocking step is used to reduce non-specific binding of target particles and molecules to the inner surface and within the pore structure of the hollow fiber membrane filter. Additionally, these blocking steps may also reduce fouling of the membrane filter by reducing binding of natural organic matter, including humic and fulvic acids, proteins, polysaccharides, other carbohydrates and other material that can cause in-pore membrane fouling. Blocking solutions can include proteins, surfactants, chemical dispersants, buffers, chelators, or other materials or chemicals that will be well known to those skilled in the art. A partial listing of specific additives that may be used in buffer solutions that will serve as blocking fluids includes: Tween 20, Tween 80, sodium polyphosphate, glycine, bovine serum albumin, fetal bovine serum, nutrient broth, beef extract, poloxamers, or other surfactants, proteins, chemical dispersants, or other chemicals that are known to improving recovery of target particles from membrane filters. These materials or chemicals may be included alone or in combination in phosphate buffer, phosphate buffered saline, or other buffer solutions that will be well known to those skilled in the art. Concentrations of the individual materials or chemicals may generally range from 0.0005% to 10% or more and may be alone or in combination with other materials or chemicals. The blocking fluids may also be heated or cooled before being pushed into CPT 2901 using heating or cooling jackets around the blocking fluid bottle or a heated or chilled fluid line.

To perform the blocking step, permeate valve 2904 and permeate pressure valve 2905 remain closed, allowing the permeate 2966 side of CPT 2901 to be sealed off from atmosphere. Foam elution valve 2932 is closed and remains closed except when wet foam is being dispensed during other operations. The distribution valve 2906 is positioned to connect port 2911 to central port 2908 and syringe pump 2907. Syringe pump 2907 is then activated to draw air from air inlet 2917 through high efficiency particle arresting (HEPA) filter 2918, fluid line 2919, port 2911, central port 2908 and then into the syringe pump 2907. A volume of air slightly more than the internal volume of fluid line 2920 is drawn. The air drawn into the syringe will later be used to push the blocking agent out of fluid line 2920 and the internal retentate flow path of CPT 2901.

Rotary distribution valve 2906 is positioned so that port 2914 is connected to central port 2908. Syringe pump 2907 is then used to draw blocking fluid 2928 from solution bottle 2929 through sterile filter 2930, through sample addition fluid line 2931, and then into syringe pump 2907. The syringe pump 2907 draw volume can range from slightly less to slightly more than the total internal volume of hollow fiber membrane filters 2926 in CPT 2901. The draw volume is set such that the blocking fluid 2928 volume is sufficient to completely fill the internal retentate volume of CPT 2901.

After the blocking fluid 2928 is drawn into syringe pump 2907 distribution valve 2906 is positioned so that syringe pump 2907 and central port 2908 are connected to port 2910. Syringe pump 2907 is then activated to dispense blocking fluid 2928 through fluid line 2920, check valve 2959, and into CPT 2901 through CPT retentate port 2902. Blocking fluid 2928 flows through check valve 2925 and down the lumen of the hollow fiber membrane filters 2926 within CPT 2901 and the syringe pump is stopped such that the leading front of the blocking fluid 2928 is stopped within the portion of hollow fibers 2926 contained within the lower potting material 2933. The combined volume of blocking fluid 2928 and air pushed by syringe pump 2907 is equivalent or roughly equivalent to the combined internal volume of fluid line 2920 and the retentate flow path of CPT 2901 minus the internal volume of the section of the hollow fiber membrane filters 2926 contained within lower potting 2933. This volume can be calculated based on the calculated internal volume of fluid line 2920 and the CPT 2901 retentate flow path. Alternatively, this volume may be empirically determined by observing the volume of fluid push that causes blocking fluid 2928 to be dispensed out of CPT opening 2927 and reducing this volume by the calculated internal volume of the hollow fiber membrane filters 2926 contained within lower potting 2933. Using these methods, the leading edge of the blocking fluid 2928 within the hollow fiber membrane filters 2926 is stopped slightly before being dispensed out of CPT opening 2927.

The flowrate that syringe 2907 dispenses the blocking fluid 2928 can be adjusted based on the number and length of hollow fiber membrane filters 2926 and the pressure drop associated with the fluid line 2920, check valve 2959, and check valve 2925. The flowrate is optimized to ensure that sufficient pressure drop is produced at the top end of the hollow fiber membrane filters 2926, so that relatively equivalent volumes are pushed into each hollow fiber.

After injection, the blocking fluid 2928 is left to incubate in the tip for a period of time generally ranging from 1 minute up to as much as 60 minutes, or more specifically from 2 minutes to 15 minutes. Some blocking fluids may not require an incubation step and the system can immediately proceed to the next step of the blocking treatment.

After the incubation period has passed the blocking fluid 2928 may either be pushed out of CPT 2901 through CPT opening 2925 and into the sample, if desirable, or into a waste container or it may be drawn through the hollow fiber membrane filter 2926 wall and into the permeate side 2966 fluidics. If dispensing into the sample or into a waste container the permeate 2966 is first brought to atmospheric pressure or positive pressure may be applied. Syringe 2907 is then driven until all fluid and air within is expelled. In this way, the air in syringe 2907 is used to push blocking fluid 2928 fully through fluid line 2920 and the internal retentate flow path of CPT 2901 and out CPT opening 2925 and into the sample or waste container.

If the blocking fluid 2928 is to be drawn through the hollow fiber membrane filters 2926 and into the permeate 2966 then permeate valve 2904 is opened and permeate pump 2947 is activated. The blocking fluid 2928 is then drawn through the hollow fiber membrane filters 2926 and into the permeate 2966. The blocking fluid 2928 then flows out through permeate path 2945 fluid line 2944 and 2951, permeate valve 2904, fluid line 2950, flow meter 2953, pressure transducer 2952, fluid line 2949, permeate pump 2947 and is dispensed to waste through fluid line 2948. In most configurations, however, some blocking fluid will remain in fluid line 2920 and in the top portions of CPT 2901, including the hollow fiber membrane filter 2926 portions contained within the upper potting. To eliminate this fluid prior to processing the sample, syringe 2907 is driven until air remaining in the syringe 2907 is able to push fluid remaining in fluid line 2920 and the top portions of CPT 2901 out and into the unpotted portions of the hollow fiber membrane filter 2926. The amount of air volume dispensed by syringe 2907 can be calculated based on the initial blocking fluid 2928 and air volume drawn into syringe 2907 and the volume dispensed from syringe 2907 and the internal volume of fluid line 2920 and the retentate flow path of CPT 2901 up to the lower edge of the upper potting. Once the remaining blocking fluid 2928 is in contact with the hollow fiber membrane filter 2926 between the upper potting 2954 and lower potting 2933 residual vacuum is able to draw the fluid through and into the permeate 2966. Additional vacuum can be applied as necessary using the permeate pump 2947. The point at which all of the blocking fluid 2928 has been drawn into the permeate 2966 is detected by using the permeate flow meter 2953 or by looking for a sudden rapid increase in permeate vacuum as measured by the permeate pressure transducer or in the pump rate when using a vacuum feedback loop. Wash steps, as will be described later, can be performed following membrane filter blocking if it is necessary to remove residual block fluid 2928.

After membrane filter blocking is complete, the sample can then be processed. A surfactant is generally added to most samples prior to processing to improve target particle recovery. The surfactant can be added when the sample is collected or by the laboratory or automatically using the sample additions steps, as described above. Tween 20 at a concentration of 1% to 20% or more specifically between 5% and 15% is routinely added as a 1:100 dilution to the sample to commonly yield a concentration of 0.05% to 0.15% Tween 20 in the sample. More broadly, final Tween 20 concentrations from 0.005% to as much as 5% or more may be used in certain applications as will be understood by those skilled in the art. Additionally, ionic fluids, chelators, dispersants or other additives at a range of pH may also be added to samples to modify zeta potential, disaggregate or inhibit aggregation, solubilize, aggregate, or modify in other ways to improve capture or passage of the certain sample components and to improve recovery of target particles from the membrane filter during the elution process. Additionally sample additions may be used to modify the streaming potential of the membrane filter to help in reducing fouling.

Processing parameters are set by the user prior to initiating sample injection (of used), membrane filter blocking (if used) or sample processing. The sample processing parameters may include the transmembrane pressure or the sample flow rate or the system may simply process the samples by drawing at the highest rate possible with the permeate pump 2947 used. Optimal processing is generally achieved by maintaining a transmembrane pressure, so that approach will be used in the following descriptions. More specifically, in the exemplary embodiment the permeate pressure transducer will be used to measure the negative pressure in the permeate fluidics. This negative pressure is equivalent to the transmembrane pressure because no pressure is applied to the retentate side of the hollow fiber membrane filters 2926 in CPT 2901. During processing an algorithm is used to control the permeate pump 2947 rate based on feedback from the permeate pressure transducer 2952. Using lower and controlled permeate pressure with the CP has been demonstrated to improve target recovery efficiency and reduce membrane filter fouling.

To initiate the sample processing CPT 2901 is first connected to the processing instrument by inserting retentate port 2902 and permeate port into the appropriate ports on the instrument. The CPT 2901 is then lowered into the sample such that CPT opening 2927 is below the fluid level of the sample. In many cases CPT opening is positioned at the bottom of the sample reservoir, but with some sample matrixes it may be advantageous for the CPT to be positioned near the top of the fluid initially and lowered slowly as the sample is processed, so that fouling materials that may precipitate out are able to be avoided until later in the processing run. In automated systems the fluid level can be detected and the tip can be automatically lowered farther into the sample as the sample is processed. Non-contact fiber optic, photoelectric, and laser sensors in the fluidics head of the described device can enable detection of the fluid height and be used to control robotics to maintain the CPT opening 2927 a set distance below the fluid level.

At the start of sample processing, permeate valve 2904 is closed and vent valve 2946 and permeate pressure valve 2905 are opened to ensure that permeate 2966 is returned to atmospheric pressure. Permeate vent valve 2946 and permeate pressure valve are then all closed, allowing the permeate 2966 side of CPT 2901 to be sealed off from atmosphere. Foam elution valve 2932 is closed and remains closed except when wet foam is being dispensed during other operations. The distribution valve 2906 is positioned to connect port 2911 to central port 2908 and syringe pump 2907. Syringe pump 2907 is then activated to draw air from air inlet 2917 through high efficiency particle arresting (HEPA) filter 2918, fluid line 2919, port 2911, central port 2908 and then into the syringe pump 2907. A volume of air slightly more than the internal volume of fluid line 2920 is drawn. The air drawn into the syringe will later be used to push wash fluid 2955 out of fluid line 2920, check valve 2959, and the internal retentate flow path of CPT 2901.

Rotary distribution valve 2906 is then positioned so that port 2913 is connected to central port 2908. Syringe pump 2907 is then used to draw wash fluid 2955 from solution bottle 2956 through sterile filter 2957, through wash fluid line 2958, and then into syringe pump 2907. The syringe pump 2907 draw volume can range from slightly less than the total internal volume of hollow fiber membrane filters 2926 in CPT 2901 to significantly larger volumes. While the smaller volume will only enable startup of sample process the larger volumes will enable both startup and also subsequent fouling restoration steps and wash steps that can be performed prior to elution. In the exemplary embodiment the draw volume is set such that the wash fluid 2955 volume is sufficient to completely fill the internal retentate volume of CPT 2901 plus enough fluid to perform a number of fouling reduction and wash steps.

To inject the wash fluid 2955, rotary distribution valve 2906 is positioned so that port 2910 is connected to central port 2908. Syringe 2907 is then activated to dispense wash fluid 2955 into fluid line 2920, through check valve 2959 and into CPT 2901 through retentate port 2902. Syringe 2907 continues to push wash fluid 2955 until the leading edge of the wash fluid 2955 reaches between the upper and lower edges of the lower potting material.

Permeate valve 2904 is then opened and permeate pump 2947 is turned on and wash fluid 2955 is then drawn through the hollow fiber membrane filters 2926 and into the permeate 2966. As wash fluid 2955 is drawn through the hollow fiber membrane filters 2926, the sample is aspirated into CPT opening 2927 and hollow fiber membrane filters 2926. The wash fluid 2955 and the sample then flow out through permeate path 2945 fluid line 2944 and 2951, permeate valve 2904, fluid line 2950, flow meter 2953, pressure transducer 2952, fluid line 2949, permeate pump 2947 and are dispensed to waste through fluid line 2948. While being processed an algorithm and feedback loop with the pressure transducer 2952 is used to control the permeate pump 2947 flowrate. Flow meter 2953 may also be used to measure the flowrate through the tip.

As the sample is processed, if the flowrate, as determined by the flow meter 2953, falls below a certain preset level or percentage of the starting flow rate, or if the power fed to permeate pump 2947 or the permeate pump 2947 RPM falls below a certain preset level (due to the algorithm control reducing the power to the permeate pump 2947) then a process for reverse fouling to the membrane filter is automatically initiated. In a exemplary embodiment a process similar to oscillating tangential flow, which will be well known to those skilled in the art, is performed in a single-use CPT without contacting sample with the processing instrument. To perform the process, the permeate pressure chamber is pressurized to a preset pressure. Syringe 2907 already contains wash fluid and fluid line 2920 through the internal retentate flow path and into the hollow fiber membrane filters is already full of wash fluid. This fluid will be used for tangential flushes during the fouling reduction steps. Permeate pump 2947 is turned off and permeate valve 2904 is closed and permeate pressure valve 2905 is opened for a short period of time in the range of 5 milliseconds to 5 seconds or more preferably in the range of 25 milliseconds to 1 second. The preset pressure used is generally in the range of 0.1 psi to 30 psi or more preferably in the range of 0.5 psi to 10 psi. Alternatively permeate pressure valve 2905 may be opened and then vent valve 2946 may be opened to return the permeate to atmospheric pressure before permeate pressure valve 2905 is closed. A considerable number of valve types and configurations can be used to achieve a short period of pressure applied to the permeate 2966 as described above. These possible approaches will be well understood by those skilled in the art after consideration of the present disclosure.

In coordination with the application of permeate back pressure a tangential flush of wash fluid 2955 is also injected out of the syringe 2907, forcing wash fluid 2955 into the top of CPT 2901 through retentate port 2902. This injection is generally performed slightly after initiation of the permeate back pressure, but while some back pressure still remains on the permeate 2966. Preferably the injection may take place at the same time or up to 5 seconds after initiation of the back pressure or more preferably between 5 milliseconds and 1 second after initiation of the back pressure. The injection will be performed with preferably between 10 μL and 1 mL of wash fluid 2955 when using current standard InnovaPrep CPTs which contain nominally 2972 hollow fiber membrane filters—producing a membrane surface area of 98 cm 2. The wash fluid 2955 is pushed out of the syringe 2907 in a very short period of time—thus creating a high flowrate push of wash fluid 2955 into CPT 2901. More preferably the wash fluid injection will be performed with between 50 μL and 700 μL Due to the backpressure remaining on the permeate, the wash fluid 2955 is forced to travel tangentially in the hollow fiber membrane filter 2926 lumen, and in the opposite direction to the normal flow during sample processing. Alternatively, to initiation the fouling reduction steps based on the flowrate setpoints the steps can also be performed on a set time basis or a combination of the two.

When the entire sample has been processed the end of run is detected using either the permeate flow meter 2953 or the permeate pressure transducer 2952 or a combination of the two. More specifically the permeate flow meter 2953 output can be used along with an algorithm to determine when the hollow fiber membrane filter 2926 has locked up due to sample no longer being drawn into the permeate and through the permeate fluidics. Alternatively, an algorithm can be used along with the pressure transducer 2952 output to look for rapid change in the permeate negative pressure (becoming more negative). This change is caused by the hollow fiber membrane filter 2926 locking up and the permeate pump 2947 continuing to draw from the permeate.

When the end of run is detected permeate valve 2904 is immediately closed and permeate pump 2947 is turned off. If desired, wash steps can be automatically performed. To perform a wash step syringe 2907 is activated to push wash fluid 2955 into CPT 2901. Rotary distribution valve 2906 is already positioned so that port 2910 is connected to central port 2908. Syringe 2907 is activated to dispense wash fluid 2955 into fluid line 2920, through check valve 2959 and into CPT 2901 through retentate port 2902. Syringe 2907 continues to push wash fluid 2955 until the leading edge of the wash fluid 2955 reaches between the upper and lower edges of the lower potting material. Syringe 2907 is then stopped and permeate valve 2904 is opened and residual negative pressure (i.e., pressure below 1 atmosphere) in permeate fluidics between permeate valve 2904 and permeate pump 2947 is able to pull the wash fluid 2966 through the wall of the hollow fiber membrane filters 2926 and into permeate 2966. When stoppage of liquid flow through the hollow fiber membrane filters 2926 is detected then permeate valve 2904 is closed. Liquid flow stoppage is detected by monitoring the permeate flow meter 2953 or the permeate pressure transducer 2953. If additional negative pressure is required to pull wash fluid 2955 through then permeate pump 2947 can be activated.

In general, a user settable transmembrane pressures for sample runs and wash steps is used to control the negative pressure in the permeate 2966. In theory, transmembrane pressures can be controlled between 0 psi and 1 atmosphere or nominally 14.7 psi, however this is dependent on the pump type and size used as well as the liquid flux of the hollow fiber membrane filters 2926. More commonly transmembrane pressures will be controlled between 0.1 psi and 10 psi and more ideally, in order to reduce compaction of particles onto the membrane surface, between 0.2 psi and 5 psi.

The wash steps can include an incubation period whereby the wash fluid is left in the lumen of the hollow fiber membrane filters 2926 for a period of time ranging from 1 second to 60 minutes. More specifically, incubation periods will range from 1 second to 1 minute in length. Repeated wash steps can be performed using the procedure described. One or more fluid types can be used for wash steps by making additional solution bottles and additional distribution valve ports available.

Wash fluids can include any number of fluid types that will help wash away soluble materials and small particles that may be co-concentrated onto the hollow fiber membrane filters along with the target materials. In general the wash fluids are meant to solubilize and dissociate these non-target materials without damaging or significantly affecting the target materials. Fluids and chemical solutions capable of performing these actions will be well known to those skilled in the art. The wash fluids may include surfactant solutions, ionic solutions, ionic liquids, coagulants, buffer solutions, chelators, higher or low pH solutions, solvent solutions, or a range of other solutions may be used. This list of possible solutions is not anticipated to be all encompassing and many other solution types could be used as will be understood by one skilled in the art. The reasons for performing a wash step include, but are not limited to: causing changes to interactions between target viral, bacterial or other particles and non-target particles present in the sample; solubilizing or washing away of natural organic matter, including humic and fulvic substances, proteins, polysaccharides, and other non-target particles and molecules; improving recovery of target particles by flushing the captured particles with Tween 20, Tween 80, sodium polyphosphate, glycine or other surfactants, proteins, chemical dispersants, or other chemicals that are known to improving recovery of target particles from membrane filters; and simply improving the buffer exchange process performed on the membrane filter by allowing residual liquid on the membrane to be washed away to the permeate.

Following the wash steps, sample labeling, dying or other treatment steps, such as DNase, RNase or intercalating die treatments, can be performed. Each of these steps follows processes similar to the wash steps described above. Labeling or dying steps are generally identical to the wash steps described except that a labeling or dying solution must be pulled from a different solution bottle and through a different distribution valve before entering syringe 2907. Additionally, the incubation period and number times that the treatment solutions are pulled into and through the hollow fiber membrane filters may require adjustment to provide sufficient time to interact with the target particles.

Following the wash step or any sample labeling, dying, or treatment steps performed, the concentrated sample may be eluted or lysis steps can be performed on target materials prior to elution. Alternatively, additional wash steps can be performed after labeling, dying, other treatment or lysis fluids have been injected, incubated and then removed. The additional wash steps enable improved removal of the labeling, dying, treatment or lysis fluids prior to performing elution or other steps. This is important in many of these approaches as will be well understood by those skilled in the art. To perform a lysis step the same process as used for wash steps can be used to introduce a lysis fluid to the lumen of the hollow fiber membrane filters 2926. To perform the lysis step, any residual wash fluid 2955 in syringe 2907 is first pushed to waste by positioning distribution valve 2906 to connect port 2916 to central port 2908 and syringe pump 2907. Syringe pump 2907 is then activated and the remaining wash fluid is pushed out through waste line 2964.

During the first stage of the lysis protocol, permeate valve 2904 and permeate pressure valve 2905 remain closed, allowing the permeate 2966 side of CPT 2901 to be sealed off from atmosphere. Foam elution valve 2932 is closed and remains closed except when wet foam is being dispensed during other operations. The distribution valve 2906 is positioned to connect port 2911 to central port 2908 and syringe pump 2907. Syringe pump 2907 is then activated to draw air from air inlet 2917 through high efficiency particle arresting (HEPA) filter 2918, fluid line 2919, port 2911, central port 2908 and then into the syringe pump 2907. The volume of air pulled is dependent on the lysis process being used with more pulled in if the lysis fluid 2960 is to be pushed out of the tip and into a final sample container or less if it will be used to perform lysis within CPT 2901. The air drawn into the syringe will be used to push the lysis fluid 2960 out of fluid line 2920 and into the internal retentate flow path of CPT 2901 or through CPT 2901 and out CPT inlet 2927.

Rotary distribution valve 2906 is positioned so that port 2912 is connected to central port 2908. Syringe pump 2907 is then used to draw lysis fluid 2960 from solution bottle 2961 through sterile filter 2962, through sample addition fluid line 2963, and then into syringe pump 2907. The syringe pump 2907 draw volume can range from less than 5% to 200% of the total internal volume of hollow fiber membrane filters 2926 in CPT 2901. In the exemplary embodiment the draw volume will range from 10% to 100% of the total internal volume of hollow fiber membrane filters 2926 in CPT 2901.

In an exemplary embodiment, after the lysis fluid 2960 is drawn into syringe pump 2907 distribution valve 2906 is positioned so that syringe pump 2907 and central port 2908 are connected to port 2910. Syringe pump 2907 is then activated to dispense lysis fluid 2960 through fluid line 2920, check valve 2959, and into CPT 2901 through CPT retentate port 2902. Lysis fluid 2960 flows through check valve 2925 and down the lumen of the hollow fiber membrane filters 2926 within CPT 2901 and out of CPT opening 2927 and into a sample container. This step may be performed at high flow rate to push target materials into lower sections of the hollow fiber membrane filters 2926 or out of CPT opening 2927 or it may be performed at a slower flow rate to give increased contact time between the bulk lysis fluid 2960 and the target materials and inner surface of the membrane. These processes may be performed with the permeate at atmospheric pressure and sealed closed or with positive pressure on the permeate. The lysis fluid 2960 is pushed out of CPT 2901 using the air in syringe 2970. After pushing the entire volume of lysis fluid 2960 out of CPT 2901 an additional incubation period may be allowed. Generally, this period will range from 10 seconds to 30 minutes, but more specifically from 30 seconds to 15 minutes. The incubation period enables the lysis fluid 2960 to lyse and solubilize materials that may bind to the hollow fiber membrane filters 2926 surface and therefor can be used to improve recovery of target particles while also initiating lysis. Of note, the lysis fluid 2960 may also be heated prior to injection into CPT 2901 using onboard heated lines.

After the lysis incubation period the remaining material in the hollow fiber membrane filters 2926 is flushed out and into the same sample container. The elution process involved flushing down the lumen with carbonated wet foam while the permeate is held at atmospheric pressure and closed off by permeate pressure valve 2905 and permeate valve 2904 or positive permeate pressure is applied as described in the other operations above. When performing elution with a positive permeate pressure the positive pressure is applied to the permeate 34 immediately before or during the wet foam elution. In this way, minimal permeate fluid is pushed back through the hollow fiber membrane filters 2926 during the process. The application of pressure to the permeate 2966 during the elution process improves recovery efficiency by allowing a small amount of permeate fluid to begin to back flush through the hollow fiber membrane filters 2926 wall.

To perform the elution, elution valve 2932 is opened for a short period of time. In a exemplary embodiment the elution valve 2932 open time may range from 1 millisecond to 1 second and more specifically from 10 milliseconds to 500 milliseconds. The elution valve 2932 opening may also be controlled by performing many short pulses of opening and closing to effectively control the rate of foam elution flow 2932. An orifice or needle valve may also be included in the valve or immediately downstream of the elution valve 2932 to assist with control of the foam flow rate. When the elution valve 2932 opens carbon dioxide pressurized elution fluid containing solubilized carbon dioxide gas in elution fluid canister 2965 is pushed out through fluid line 2934 and elution valve 2932 and into CPT 2901 retentate port 2902. Check valve 2959 keeps elution fluid from traveling backwards into fluid line 2920. Alternatively, fluid line 2920 can be filled with wash fluid 2955 or other incompressible fluid to eliminate the possibility of significant elution foam traveling into fluid line 2920.

As the carbon dioxide comes out of solution at the reduced pressure a wet foam is formed due to a surfactant or other material acting as a foaming agent and due to microbubbles formed by the carbon dioxide. The wet foam travels through check valve 2925 and down the lumen of the hollow fiber membrane filters 2926 and out of CPT opening 2927 and into the sample container. In this case the final sample is a solution of lysis fluid and wet foam elution fluid. The sample container can then be incubated at room temperature or heated or bead beat to enhance lysis.

The elution fluid generally contains a buffered solution with a surfactant or other foaming agent and is held under a carbon dioxide head pressure between 30 psi and 250 psi or more preferably between 75 psi and 150 psi. Alternatively, other soluble or insoluble gasses including nitrogen, nitrous oxide and any number of other gases or combination of gasses can be used as will be well known by those skilled in the art. Foaming agents can include surfactants such as Tween 20, Tween 20, Triton X-100, SDS, Pluronic and any number of other surfactants that will be known to those skilled in the art. Additionally, alternative foam agents including proteins, growth media and other materials that will produce a foam can be used along with a surfactant or alone. Buffers, salts, chelators and other additives may also be used to maintain pH, bacterial viability, enhance recovery, improve assay compatibility, or to achieve other results as will be well understood by those skilled in the art.

The elution fluid can also be formulated to act alone as a lysis fluid or to improve lysis when combined with the lysis fluid 2960. As one example lysis fluid 2960 may contain enzymes to initiate breakdown of cell wall components and the elution fluid may contain SDS to complete lysis after an incubation step in lysis fluid 2960.

In another exemplary embodiment, after the lysis fluid 2960 is drawn into syringe pump 2907 distribution valve 2906 is positioned so that syringe pump 2907 and central port 2908 are connected to port 2910. Syringe pump 2907 is then activated to dispense lysis fluid 2960 through fluid line 2920, check valve 2959, and into CPT 2901 through CPT retentate port 2902. Lysis fluid 2960 flows through check valve 2925 and down the lumen of the hollow fiber membrane filters 2926 within CPT 2901 and the syringe pump is stopped such that the leading front of the lysis fluid 2960 is stopped within the portion of hollow fibers 2926 contained within the lower potting material 2933.

The flowrate that syringe 2907 dispenses the lysis fluid 2960 can be adjusted based on the number and length of hollow fiber membrane filters 2926 and the pressure drop associated with the fluid line 2920, check valve 2959, and check valve 2925. The flowrate is optimized to ensure that sufficient pressure drop is produced at the top end of the hollow fiber membrane filters 2926, so that relatively equivalent volumes are pushed into each hollow fiber.

After injection, the lysis fluid 2960 is left to incubate in the tip for a much shorter period of time generally ranging from 1 second up to as much as 10 minutes, or more specifically from 1 second to 1 minute.

After the incubation period has passed the lysis fluid 2960 is drawn through hollow fiber membrane filters 2926 into the permeate 2966 using a low transmembrane pressure. Permeate pressure valve 2905 is opened and permeate valve 2904 is opened and vent valve 2946 is opened to return all permeate fluidics to atmospheric pressure. The valves are then closed and permeate pump 2947 is activated and the permeate pressure is brought to between −0.1 and −5 psi or more preferably between −0.2 and −1 psi. Permeate valve 2904 is then opened and the lysis fluid 2960 then flows out through permeate path 2945 fluid line 2944 and 2951, permeate valve 2904, fluid line 2950, flow meter 2953, pressure transducer 2952, fluid line 2949, permeate pump 2947 and fluid dispensed to waste through fluid line 2948. In most configurations, however, some lysis fluid 2960 will remain in fluid line 2920 and in the top portions of CPT 2901, including the hollow fiber membrane filter 2926 portions contained within the upper potting. To eliminate this fluid prior to processing the sample, syringe 2907 is driven until air remaining in the syringe 2907 is able to push fluid remaining in fluid line 2920 and the top portions of CPT 2901 out and into the unpotted portions of the hollow fiber membrane filter 2926. The amount of air volume dispensed by syringe 2907 can be calculated based on the initial blocking fluid 2928 and air volume drawn into syringe 2907 and the volume dispensed from syringe 2907 and the internal volume of fluid line 2920 and the retentate flow path of CPT 2901 up to the lower edge of the upper potting. Once the remaining blocking fluid 2928 is in contact with the hollow fiber membrane filter 2926 between the upper potting 2954 and lower potting 2933 residual vacuum is able to draw the fluid through and into the permeate 2966. Additional vacuum can be applied as necessary using the permeate pump 2947. The point at which all of the lysis fluid 2960 has been drawn into the permeate 2966 is detected by using the permeate flow meter 2953 or by looking for a sudden rapid increase in permeate vacuum as measured by the permeate pressure transducer or in the pump rate when using a vacuum feedback loop.

Using the above described lysis process wherein the lysis fluid 60 is drawn through and into the permeate 2966 requires that the target cells, bacteria, or virus are not fully lysed if using membrane pore sizes that are too large to retain nucleic acids or other target materials associate with the cells. In the case that larger pore sizes are used then the process is optimized to enable lysis fluid to sit between the interstitial space between particles and between particles and the hollow fiber membrane filter surface. This is primarily performed by limiting the incubation period before drawing the lysis fluid into the permeate and by limiting the transmembrane pressure and time period that it is applied. In this way the lysis process can proceed and significant quantities of nucleic acids are not lost through to the permeate, but a significant buffer exchange and dilution of the lysis fluid can occur when eluted. Elution can proceed as described above following this lysis process.

In one exemplary embodiment of the subject disclosure presented in FIG. 29 check valve 2925 can be eliminated by keeping a plug of clean fluid inside of fluid line 2920 and the internal volume of retentate port 2902. Additionally, if necessary is some applications the syringe can be used to push a very slow flow rate of clean fluid into the retentate port 2902 during sample processing.

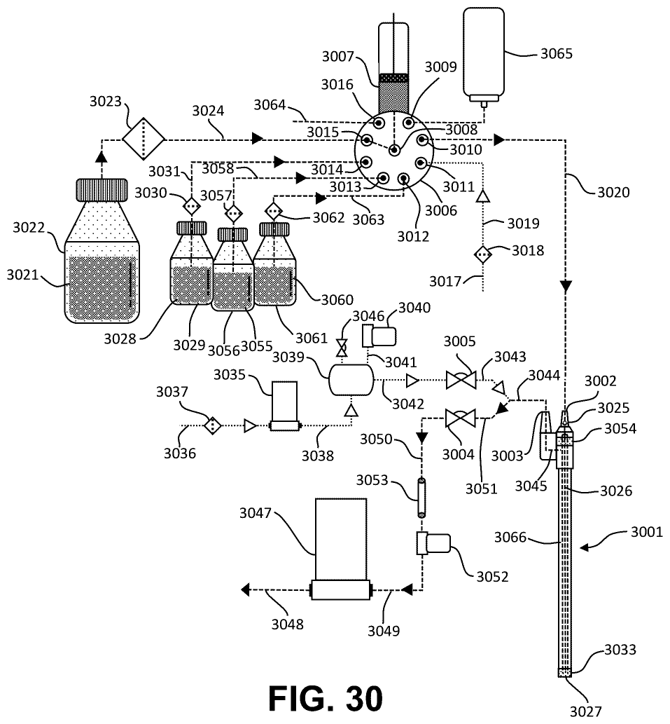

An exemplary embodiment of the present subject disclosure is shown in FIG. 30. This embodiment enables the same operations as that shown in FIG. 29, but allows for replacement of elution fluid valve 2932 with use of the syringe to measure out volumes of elution fluid prior to elution. To perform this operation the rotary distribution valve is connected to elution fluid canister and the syringe is very slowly drawn open until the desire volume of elution fluid is dispensed into the syringe. The syringe must be drawn back slowly to reduce the level of off-gassing of carbon dioxide. After drawing back the rotary valve can be turned to a closed-off port and the syringe then can be closed slightly to cause any carbon dioxide gas bubbles to go back into solution. To dispense the foam the rotary valve is turned to connect the fluid line connecting the rotary valve to the CPT to the syringe port on the valve. The elution fluid can then travel out of the syringe and create the wet foam. Off gassing in the syringe causes the foam to travel out and the syringe can also be pushed in to drive the foam and remaining liquid out.

Additionally, the embodiment presented in FIG. 30 uses two pressure transducers, one for measurement of the permeate pressure and one to measure the pressure within a permeate pressure reservoir to allow for control of the permeate back pressure by use of an algorithm to calculate the required permeate pressure reservoir pressure or the required permeate pressure valve open time. A detailed description of these approaches is provided in the description below. It is important to note that the elution foam dispensing and permeate back pressure approaches described within FIG. 29 and FIG. 30, can be used as described or each may be used with the other approaches as is desirable for the intended results.

The FIG. 30 process is especially amenable to automation of complex processes. These processes for particle manipulation include, but are not limited to concentration, wash steps, buffer exchanges, labeling, dying, lysis and a variety of other treatment steps that are routinely performed in the laboratory using automated, semi-automated, and manual treatment methods.

The manipulation or treatment steps can be selected by the user from a menu or custom methods may be used by modifying available instrument methods. After selecting the appropriate steps CPT 3001 is manually or automatically inserted into the instrument with retentate port 3002 and permeate port 3003 inserted into the appropriate ports on the instrument. Following insertion of CPT 3001 into the appropriate ports the user places the sample container holding the sample to be processed in the appropriate location under CPT 3001 or in the case of an automated system on or more samples will be placed in the appropriate locations. Generally, CPT 3001 is then lowered into the sample by lowering a movable head which holds CPT 3001 or by raising a table which holds the sample container. In some approaches the CPT 3001 may be positioned just above the sample and only lowered into the sample later in the process when the sample processing portion of the described process is performed. The described embodiment can be configured in an automated robotic manner such that it is capable of processing more than one sample can be automatically processed into a single, pooled and concentrated sample. Additionally, in a semi-automated system multiple samples can be processed with the user alerted to place the samples in the appropriate location at the required time.

The sample process can be initiated by the user by pressing the appropriate user interface controls on the instrument. As stated earlier there are a number of possible manipulation steps that can be performed using the described embodiment. Each possible manipulation process that can be performed will now be described in what is the most common order of processing. Other possible manipulations and modifications to these manipulations, as well as the order that the manipulations are performed with the described embodiment, can be modified as necessary with the described embodiment allowing for significant freedom for the operated to change the processes to achieve desired results. As one of ordinary skill in the art would appreciate, other sequences of steps may be possible.

The specific processes to be described for this exemplary embodiment include: injection of sample additive into the sample, membrane blocking, sample processing, membrane filter fouling treatment, sample washing, sample treatment, sample elution for treatment outside of tip prior to reprocessing in the tip, sample labeling, sample dying, and elution into a concentrated sample. As stated earlier this exemplary embodiment enables manipulations of samples in a number of ways and the list provided is not anticipated to be all encompassing as will be understood by one skilled in the art.

In some applications there may be advantages to adding additives to the sample being processed. These additives may be surfactant solutions, ionic solutions, coagulants, buffer solutions, chelators, higher or low pH solutions meant to change the pH of the sample, a solvent solution, or a range of other solutions that can be used to modify the sample in some way. This list of possible solutions is not anticipated to be all encompassing and many other solution types could be used as will be understood by one skilled in the art. The reasons for performing a sample addition step include, but are not limited to: causing changes to interactions between target viral, bacterial or other particles and non-target particles present in the sample; changing pH or other sample characteristics to modify the solubility of natural organic matter, including humic and fulvic substances, proteins, polysaccharides, and other target and non-target particles and molecules; modify particle zeta potential through fluid ionic strength changes or additives for enhancing steric hinderance; improving recovery of target particles by adding Tween 20, Tween 80, sodium polyphosphate, glycine or other surfactants, proteins, chemical dispersants, or other chemicals that are known to improving recovery of target particles from membrane filters; addition of DNase or RNase to remove free DNA; free RNA or DNA or RNA associated with cellular debris; addition of intercalating dyes such as propidium monoazide or ethidium monoazide to bind free DNA; addition of coagulants or flocculants such as for removing natural organic matter including humic or fulvic acids or other organic material such as proteins, polysaccharides, other carbohydrates, or any other number of materials that may cause in pore fouling. Coagulants and flocculants may be selected from aluminum-based coagulants, ferric-based coagulants, polymeric flocculants, organic polyelectrolytes, and other available coagulants and flocculants that will be well known to those skilled in the art. Many other sample additions that will be known to those skilled in the art may also be added using the described approach.

To inject a sample additive into the sample, permeate valve 3004 and permeate pressure valve 3005 are closed, allowing the permeate 3066 of CPT 3001 to be sealed off from atmosphere. The multiport rotary distribution valve 3006 is used to connect syringe pump 3007 to one of six ports through central port 3008. The distribution valve 3006 is first positioned to connect port 3011 to central port 3008 and syringe pump 3007. Syringe pump 3007 is then activated to draw air from air inlet 3017 through high efficiency particle arresting (HEPA) filter 3018, fluid line 3019, port 3011, central port 3008 and then into the syringe 3007. A volume of air equivalent to or slightly more than the internal volume of fluid line 3020 plus the internal retentate flow path through CPT 3001 is drawn. The air drawn into the syringe will later be used to flush sampled additive out of fluid line 3020 and the internal retentate flow path of CPT 3001.

Next, rotary distribution valve 3006 is positioned so that port 3015 is connected to central port 3008. Syringe pump 3007 is then used to draw sample addition fluid 3021 from solution bottle 3022 through sterile filter 3023, sample addition fluid line 3024, and then into syringe pump 3007. The syringe pump 3007 draw volume is equivalent to the required sample addition volume.

After the sample addition fluid 3021 is drawn into syringe pump 3007 distribution valve 3006 is positioned so that syringe pump 3007 and central port 3008 are connected to port 3010. Syringe pump 3007 is then activated to dispense sample addition fluid 3021 through fluid line 3020 and into CPT 3001 through CPT retentate port 3002. Sample addition fluid 3021 flows through check valve 3025 and down the lumen of the hollow fiber membrane filters 3026 within CPT 3001 and out through CPT opening 3027 and into the sample. In this configuration CPT 3001 can be used with or without check valve 3025. Syringe 3007 is driven until all fluid and air within is expelled. In this way, the air in syringe 3007 is used to push sample addition fluid 3021 fully through fluid line 3020, check valve 3059, and the internal retentate flow path of CPT 3001 and out CPT opening 3027 and into the sample.

The flowrate that syringe 3007 dispenses the sample addition fluid 3021 at can be adjusted based on the number and length of hollow fiber membrane filters 3026 and the pressure drop associated with the fluid line 3020 and check valve 3025. The flowrate is optimized to ensure that sufficient pressure drop is produced at the top end of the hollow fiber membrane filters 3026, so that relatively equivalent volumes are pushed into each hollow fiber. Further, the flowrate is optimized to reduce the flow of sample addition fluid 3021 through the wall of hollow fiber membrane filters 3026 and into the permeate 3066. Additionally, slight positive pressure can be applied to the permeate 3066 to reduce the passage of sample addition fluid 3021 into the permeate 3066.

For clarity, the retentate of CPT 3001 is made up of the internal flow path of the hollow fiber membrane filters 3026 plus the interior flow path within CPT retentate port 3002 that is fluidically connected to the internal volume of hollow fiber membrane filters 3026. Permeate 3066 is made up of the area between the outer wall of hollow fiber membrane filters 3026 and the wall of CPT 3001 and enclosed between potting 3033 and 3054. Permeate 3066 is fluidically connected to permeate path 3045.

To apply positive backpressure, air pump 3035 is first activated and air is drawn in through air inlet 3036 through HEPA filter 3037. The air is pushed through fluid line 3038 and into pressure chamber 3039 until a preset or algorithm calculated pressure is reached according to pressure transducer 3040 which is fluidically connected to pressure chamber 3039 via fluid line 3041 or until a required pressure. The system controls may use an algorithm to calculate a required permeate pressure that is needed to reach a user set permeate backpressure. This algorithm uses the measured transmembrane pressure and known pressure reservoir and permeate volumes to calculate the needed pressure.

With air pressure controlled at the preset pressure, permeate pressure valve 3005 is opened to allow pressurized air to flow through fluid line 3042, the permeate pressure valve 3005, fluid line 3043, fluid line 3044 and into CPT 3001 through permeate port 3003. Specifically, the pressurized air flows through CPT 3001 permeate path 3045 and into permeate side 3066. Permeate valve 3005 can then be closed, allowing pressure to remain on the permeate side 3066. Air pressure will normally be applied immediately after the sample addition fluid 3021 has reached CPT opening 3027. In this way the hollow fiber membrane filters 3026 are wetted out and do not readily allow air to pass through into the retentate. The positive pressure applied to the permeate 3066 is generally between 0.1 psi and 50 psi and more specifically between 0.5 psi and 5 psi. Depending on the characteristics of the hollow fiber membrane filters 3026 and sample addition fluid 3021 atmospheric pressure may be applied or slight negative pressure can be applied to the permeate 3066 for a short period of time before positive pressure is applied to ensure that the hollow fiber membrane filters 3026 fully wet out and do not allow the pressurized air to pass.

If a lower permeate chamber 3039 pressure is required at any point then atmospheric pressure can be applied by opening pressure vent valve 3046. A HEPA or other vent filter may be used on the open side of vent valve 3046 to reduce the potential for introduction of particles from atmospheric air.

After applying pressure, pressure valve 3005 may then be closed if sealing off of the permeate side 3066 is desired. Negative pressure can be applied to the permeate 3066 by closing permeate pressure valve 3005, opening permeate valve 3004, and turning on permeate pump 3047. The pump will then apply negative pressure to the permeate 3066 by pushing air or fluid out through fluid line 3048 while pulling air through fluid lines 3049, 3050, 3051, and 3044 and through permeate valve 3004 and permeate path 3045. The negative pressure on the permeate can be measured and controlled during this process by using pressure transducer 3052 and permeate pump 3047. The flowrate through the permeate can also be measured using permeate flow meter 3053.

A number of similar configurations can be used for applying negative, atmospheric or positive pressure to the permeate 3066 as will be understand by those skilled in the art. These configurations can include the use of number of different types and styles of check valves or electronically controlled valves to enable connection to a vacuum pump or to a pressure reservoir or atmospheric vent. Further use of pressure transducers and flow meters enable control of these parameters in the system.

One or more sample additional fluids can be added to the sample using the described approach if multiport valves with more ports and additional bottles are connected to the system. The sample addition fluids may also be heated or cooled before addition to the sample by heating or cooling jackets around the sample additional fluid bottle or using a heated or chilled fluid line. Following sample addition, the sample with additive can be subjected to additional treatment steps such as vortexing, stirring with a magnetic stir bar or other stirring mechanism, shaking, heating, cooling, sonication, bead beating or any number of other treatments that will be known to those skilled in the art. Additionally, using a similar approach to that described for sample additive addition, air can be pushed through CPT 3001 and into the sample as a method of mixing the sample. The sample may also be left at room temperature or a heated or cooled temperature for a period of time, as may be beneficial with certain additives, including surfactants and dispersants, and other buffers, as will be well known to those skilled in the art, before proceeding with sample processing.

After the sample additive has been pushed out of CPT 3001 a membrane filter blocking step can be performed. The blocking step is used to reduce binding of target particles and molecules to the inner surface and within the pore structure of the hollow fiber membrane filter. Additionally, these blocking steps may also reduce fouling of the membrane filter by reducing binding of natural organic matter, including humic and fulvic acids, proteins, polysaccharides, other carbohydrates and other material that can cause in-pore membrane fouling. Blocking solutions can include proteins, surfactants, chemical dispersants, buffers, chelators, or other materials or chemicals that will be well known to those skilled in the art. A partial listing of specific additives that may be used in buffer solutions that will serve as blocking fluids includes: Tween 20, Tween 80, sodium polyphosphate, glycine, bovine serum albumin, fetal bovine serum, nutrient broth, beef extract, poloxamers, or other surfactants, proteins, chemical dispersants, or other chemicals that are known to improving recovery of target particles from membrane filters. These materials or chemicals may be included alone or in combination in phosphate buffer, phosphate buffered saline, or other buffer solutions that will be well known to those skilled in the art. Concentrations of the individual materials or chemicals may generally range from 0.0005% to 10% or more and may be alone or in combination with other materials or chemicals. The blocking fluids may also be heated or cooled before being pushed into CPT 3001 using heating or cooling jackets around the blocking fluid bottle or a heated or chilled fluid line.

To perform the blocking step, permeate valve 3004 and permeate pressure valve 3005 remain closed, allowing the permeate 3066 side of CPT 3001 to be sealed off from atmosphere. Foam elution valve 3032 is closed and remains closed except when wet foam is being dispensed during other operations. The distribution valve 3006 is positioned to connect port 3011 to central port 3008 and syringe pump 3007. Syringe pump 3007 is then activated to draw air from air inlet 3017 through high efficiency particle arresting (HEPA) filter 3018, fluid line 3019, port 3011, central port 3008 and then into the syringe pump 3007. A volume of air slightly more than the internal volume of fluid line 3020 is drawn. The air drawn into the syringe will later be used to push the blocking agent out of fluid line 3020 and the internal retentate flow path of CPT 3001.

Rotary distribution valve 3006 is positioned so that port 3014 is connected to central port 3008. Syringe pump 3007 is then used to draw blocking fluid 3028 from solution bottle 3029 through sterile filter 3030, through sample addition fluid line 3031, and then into syringe pump 3007. The syringe pump 3007 draw volume can range from slightly less to slightly more than the total internal volume of hollow fiber membrane filters 3026 in CPT 3001. The draw volume is set such that the blocking fluid 3028 volume is sufficient to completely fill the internal retentate volume of CPT 3001.

After the blocking fluid 3028 is drawn into syringe pump 3007 distribution valve 3006 is positioned so that syringe pump 3007 and central port 3008 are connected to port 3010. Syringe pump 3007 is then activated to dispense blocking fluid 3028 through fluid line 3020 and into CPT 3001 through CPT retentate port 3002. Blocking fluid 3028 flows through check valve 3025 and down the lumen of the hollow fiber membrane filters 3026 within CPT 3001 and the syringe pump is stopped such that the leading front of the blocking fluid 3028 is stopped within the portion of hollow fibers 3026 contained within the lower potting material 3033. The combined volume of blocking fluid 3028 and air pushed by syringe pump 3007 is equivalent or roughly equivalent to the combined internal volume of fluid line 3020 and the retentate flow path of CPT 3001 minus the internal volume of the section of the hollow fiber membrane filters 3026 contained within lower potting 3033. This volume can be calculated based on the calculated internal volume of fluid line 3020 and the CPT 3001 retentate flow path. Alternatively, this volume may be empirically determined by observing the volume of fluid push that causes blocking fluid 3028 to be dispensed out of CPT opening 3027 and reducing this volume by the calculated internal volume of the hollow fiber membrane filters 3026 contained within lower potting 3033. Using these methods the leading edge of the blocking fluid 3028 within the hollow fiber membrane filters 3026 is stopped slightly before being dispensed out of CPT opening 3027.

The flowrate that syringe 3007 dispenses the blocking fluid 3028 can be adjusted based on the number and length of hollow fiber membrane filters 3026 and the pressure drop associated with the fluid line 3020, check valve 3059, and check valve 3025. The flowrate is optimized to ensure that sufficient pressure drop is produced at the top end of the hollow fiber membrane filters 3026, so that relatively equivalent volumes are pushed into each hollow fiber.

After injection, the blocking fluid 3028 is left to incubate in the tip for a period of time generally ranging from 1 minute up to as much as 60 minutes, or more specifically from 2 minutes to 15 minutes. Some blocking fluids may not require an incubation step and the system can immediately proceed to the next step of the blocking treatment.

After the incubation period has passed the blocking fluid 3028 may either be pushed out of CPT 3001 through CPT opening 3025 and into the sample, if desirable, or into a waste container or it may be drawn through the hollow fiber membrane filter 3026 wall and into the permeate side 3066 fluidics. If dispensing into the sample or into a waste container the permeate 3066 is first brought to atmospheric pressure or positive pressure may be applied. Syringe 3007 is then driven until all fluid and air within is expelled. In this way, the air in syringe 3007 is used to push blocking fluid 3028 fully through fluid line 3020 and the internal retentate flow path of CPT 3001 and out CPT opening 3025 and into the sample or waste container.

If the blocking fluid 3028 is to be drawn through the hollow fiber membrane filters 3026 and into the permeate 3066 then permeate valve 3004 is opened and permeate pump 3047 is activated. The blocking fluid 3028 is then drawn through the hollow fiber membrane filters 3026 and into the permeate 3066. The blocking fluid 3028 then flows out through permeate path 3045 fluid line 3044 and 3051, permeate valve 3004, fluid line 3050, flow meter 3053, pressure transducer 3052, fluid line 3049, permeate pump 3047 and is dispensed to waste through fluid line 3048. In most configurations, however, some blocking fluid will remain in fluid line 3020 and in the top portions of CPT 3001, including the hollow fiber membrane filter 3026 portions contained within the upper potting. To eliminate this fluid prior to processing the sample, syringe 3007 is driven until air remaining in the syringe 3007 is able to push fluid remaining in fluid line 3020 and the top portions of CPT 3001 out and into the unpotted portions of the hollow fiber membrane filter 3026. The amount of air volume dispensed by syringe 3007 can be calculated based on the initial blocking fluid 3028 and air volume drawn into syringe 3007 and the volume dispensed from syringe 3007 and the internal volume of fluid line 3020 and the retentate flow path of CPT 3001 up to the lower edge of the upper potting. Once the remaining blocking fluid 3028 is in contact with the hollow fiber membrane filter 3026 between the upper potting 3054 and lower potting 3033 residual vacuum is able to draw the fluid through and into the permeate 3066. Additional vacuum can be applied as necessary using the permeate pump 3047. The point at which all of the blocking fluid 3028 has been drawn into the permeate 3066 is detected by using the permeate flow meter 3053 or by looking for a sudden rapid increase in permeate vacuum as measured by the permeate pressure transducer or in the pump rate when using a vacuum feedback loop. Wash steps, as will be described later, can be performed following membrane filter blocking if it is necessary to remove residual block fluid 3028.

After membrane filter blocking is complete the sample can then be processed. A surfactant is generally added to most samples prior to processing to improve target particle recovery. The surfactant can be added when the sample is collected or by the laboratory or automatically using the sample additions steps, as described above. Tween 20 at a concentration of 1% to 20% or more specifically between 5% and 15% is routinely added as a 1:100 dilution to the sample to commonly yield a concentration of 0.05% to 0.15% Tween 20 in the sample. More broadly, final Tween 20 concentrations from 0.005% to as much as 5% or more may be used in certain applications as will be understood by those skilled in the art. Additionally, ionic fluids, chelators, dispersants or other additives at a range of pH may also be added to samples to modify zeta potential, disaggregate or inhibit aggregation, solubilize, aggregate, or modify in other ways to improve capture or passage of the certain sample components and to improve recovery of target particles from the membrane filter during the elution process. Additionally sample additions may be used to modify the streaming potential of the membrane filter to help in reducing fouling.

Processing parameters are set by the user prior to initiating sample injection (of used), membrane filter blocking (if used) or sample processing. The sample processing parameters may include the transmembrane pressure or the sample flow rate or the system may simply process the samples by drawing at the highest rate possible with the permeate pump 3047 used. Optimal processing is generally achieved by maintaining a transmembrane pressure, so that approach will be used in the following descriptions. More specifically, in the exemplary embodiment the permeate pressure transducer will be used to measure the negative pressure in the permeate fluidics. This negative pressure is equivalent to the transmembrane pressure because no pressure is applied to the retentate side of the hollow fiber membrane filters 3026 in CPT 3001. During processing an algorithm is used to control the permeate pump 3047 rate based on feedback from the permeate pressure transducer 3052. Using lower and controlled permeate pressure with the CP has been demonstrated to improve target recovery efficiency and reduce membrane filter fouling.

To initiate the sample processing CPT 3001 is first connected to the processing instrument by inserting retentate port 3002 and permeate port into the appropriate ports on the instrument. The CPT 3001 is then lowered into the sample such that CPT opening 3027 is below the fluid level of the sample. In many cases CPT opening is positioned at the bottom of the sample reservoir, but with some sample matrixes it may be advantageous for the CPT to be positioned near the top of the fluid initially and lowered slowly as the sample is processed, so that fouling materials that may precipitate out are able to be avoided until later in the processing run. In automated systems the fluid level can be detected and the tip can be automatically lowered farther into the sample as the sample is processed. Non-contact fiber optic, photoelectric, and laser sensors in the fluidics head of the described device can enable detection of the fluid height and be used to control robotics to maintain the CPT opening 3027 a set distance below the fluid level.

At the start of sample processing permeate valve 3004 and permeate pressure valve 3005 are closed. Foam elution valve 3032 is closed and remains closed except when wet foam is being dispensed during other operations. The distribution valve 3006 is positioned to connect port 3011 to central port 3008 and syringe pump 3007. Syringe pump 3007 is then activated to draw air from air inlet 3017 through high efficiency particle arresting (HEPA) filter 3018, fluid line 3019, port 3011, central port 3008 and then into the syringe pump 3007. A volume of air slightly more than the internal volume of fluid line 3020 is drawn. The air drawn into the syringe will later be used to push wash fluid 3055 out of fluid line 3020, check valve 3059, and the internal retentate flow path of CPT 3001.

Rotary distribution valve 3006 is then positioned so that port 3013 is connected to central port 3008. Syringe pump 3007 is then used to draw wash fluid 3055 from solution bottle 3056 through sterile filter 3057, through wash fluid line 3058, and then into syringe pump 3007. The syringe pump 3007 draw volume can range from slightly less than the total internal volume of hollow fiber membrane filters 3026 in CPT 3001 to significantly larger volumes. While the smaller volume will only enable startup of sample process the larger volumes will enable both startup and also subsequent fouling restoration steps and wash steps that can be performed prior to elution. In the exemplary embodiment the draw volume is set such that the wash fluid 3055 volume is sufficient to completely fill the internal retentate volume of CPT 3001 plus enough fluid to perform a number of fouling reduction and wash steps.

To inject the wash fluid 3055, rotary distribution valve 3006 is positioned so that port 3010 is connected to central port 3008. Syringe 3007 is then activated to dispense wash fluid 3055 into fluid line 3020, through check valve 3059 and into CPT 3001 through retentate port 3002. Syringe 3007 continues to push wash fluid 3055 until the leading edge of the wash fluid 3055 reaches between the upper and lower edges of the lower potting material.

Permeate valve 3004 is then opened and permeate pump 3047 is turned on and wash fluid 3055 is then drawn through the hollow fiber membrane filters 3026 and into the permeate 3066. As wash fluid 3055 is drawn through the hollow fiber membrane filters 3026, the sample is aspirated into CPT opening 3027 and hollow fiber membrane filters 3026. The wash fluid 3055 and the sample then flow out through permeate path 3045 fluid line 3044 and 3051, permeate valve 3004, fluid line 3050, flow meter 3053, pressure transducer 3052, fluid line 3049, permeate pump 3047 and are dispensed to waste through fluid line 3048. While being processed an algorithm and feedback loop with the pressure transducer 3052 is used to control the permeate pump 3047 flowrate. Flow meter 3053 may also be used to measure the flowrate through the tip.

As the sample is processed, if the flowrate, as determined by the flow meter 3053, falls below a certain preset level or percentage of the starting flow rate, or if the power fed to permeate pump 3047 or the permeate pump 3047 RPM falls below a certain preset level (due to the algorithm control reducing the power to the permeate pump 3047) then a process for reverse fouling to the membrane filter is automatically initiated. In a exemplary embodiment a process similar to oscillating tangential flow, which will be well known to those skilled in the art, is performed in a single-use CPT without contacting sample with the processing instrument. To perform the process, the permeate pressure chamber is pressurized to a pressure calculated by an algorithm to provide a desired back pressure based on the preset permeate pressure and the internal volume of the back pressure system and the permeate system. Syringe 3007 already contains wash fluid and fluid line 3020 through the internal retentate flow path and into the hollow fiber membrane filters is already full of wash fluid. This fluid will be used for tangential flushes during the fouling reduction steps. Permeate pump 3047 is turned off and permeate valve 3004 is closed and permeate pressure valve 3005 is opened for a short period of time in the range of 5 milliseconds to 5 seconds or more preferably in the range of 25 milliseconds to 1 second. The preset pressure used is generally in the range of 0.1 psi to 30 psi or more preferably in the range of 0.5 psi to 10 psi. Alternatively permeate pressure valve 3005 may be opened and then vent valve 3046 may be opened to return the permeate to atmospheric pressure before permeate pressure valve 3005 is closed. A considerable number of valve types and configurations can be used to achieve a short period of pressure applied to the permeate 3066 as described above. These possible approaches will be well understood by those skilled in the art.

In coordination with the application of permeate back pressure a tangential flush of wash fluid 3055 is also injected out of the syringe 3007, forcing wash fluid 3055 into the top of CPT 3001 through retentate port 3002. This injection is generally performed slightly after initiation of the permeate back pressure, but while some back pressure still remains on the permeate 3066. Preferably the injection may take place at the same time or up to 5 seconds after initiation of the back pressure or more preferably between 5 milliseconds and 1 second after initiation of the back pressure. The injection will be performed with preferably between 10 μL and 1 mL of wash fluid 3055 when using current standard InnovaPrep CPTs which contain nominally 72 hollow fiber membrane filters—producing a membrane surface area of 98 cm². The wash fluid 3055 is pushed out of the syringe 3007 in a very short period of time—thus creating a high flowrate push of wash fluid 3055 into CPT 3001. More preferably the wash fluid injection will be performed with between 50 μL and 700 μL Due to the backpressure remaining on the permeate, the wash fluid 3055 is forced to travel tangentially in the hollow fiber membrane filter 3026 lumen, and in the opposite direction to the normal flow during sample processing. Alternatively, to initiation the fouling reduction steps based on the flowrate setpoints the steps can also be performed on a set time basis or a combination of the two.

When the entire sample has been processed the end of run is detected using either the permeate flow meter 3053 or the permeate pressure transducer 3052 or a combination of the two. More specifically the permeate flow meter 3053 output can be used along with an algorithm to determine when the hollow fiber membrane filter 3026 has locked up due to sample no longer being drawn into the permeate and through the permeate fluidics. Alternatively, an algorithm can be used along with the pressure transducer 3052 output to look for rapid change in the permeate negative pressure (becoming more negative). This change is caused by the hollow fiber membrane filter 3026 locking up and the permeate pump 3047 continuing to draw from the permeate.

When the end of run is detected permeate valve 3004 is immediately closed and permeate pump 3047 is turned off. If desired, wash steps can be automatically performed. To perform a wash step syringe 3007 is activated to push wash fluid 3055 into CPT 3001. Rotary distribution valve 3006 is already positioned so that port 3010 is connected to central port 3008. Syringe 3007 is activated to dispense wash fluid 3055 into fluid line 3020, through check valve 3059 and into CPT 3001 through retentate port 3002. Syringe 3007 continues to push wash fluid 3055 until the leading edge of the wash fluid 3055 reaches between the upper and lower edges of the lower potting material. Syringe 3007 is then stopped and permeate valve 3004 is opened and residual negative pressure (i.e. pressure below 1 atmosphere) in permeate fluidics between permeate valve 3004 and permeate pump 3047 is able to pull the wash fluid 3066 through the wall of the hollow fiber membrane filters 3026 and into permeate 3066. When stoppage of liquid flow through the hollow fiber membrane filters 3026 is detected then permeate valve 3004 is closed. Liquid flow stoppage is detected by monitoring the permeate flow meter 3053 or the permeate pressure transducer 3053. If additional negative pressure is required to pull wash fluid 3055 through then permeate pump 3047 can be activated.

In general, a user settable transmembrane pressures for sample runs and wash steps is used to control the negative pressure in the permeate 3066. In theory, transmembrane pressures can be controlled between 0 psi and 1 atmosphere or nominally 14.7 psi, however this is dependent on the pump type and size used as well as the liquid flux of the hollow fiber membrane filters 3026. More commonly transmembrane pressures will be controlled between 0.1 psi and 10 psi and more ideally, in order to reduce compaction of particles onto the membrane surface, between 0.2 psi and 5 psi.

The wash steps can include an incubation period whereby the wash fluid is left in the lumen of the hollow fiber membrane filters 3026 for a period of time ranging from 1 second to 60 minutes. More specifically, incubation periods will range from 1 second to 1 minute in length. Repeated wash steps can be performed using the procedure described. One more fluid types can be used for wash steps by making additional solution bottles and additional distribution valve ports available.

Wash fluids can include any number of fluid types that will help wash away soluble materials and small particles that may be co-concentrated onto the hollow fiber membrane filters along with the target materials. In general the wash fluids are meant to solubilize and dissociate these non-target materials without damaging or significant affecting the target materials. Fluids and chemical solutions capable of performing these actions will be well known to those skilled in the art. The wash fluids may include surfactant solutions, ionic solutions, ionic liquids, coagulants, buffer solutions, chelators, higher or low pH solutions, solvent solutions, or a range of other solutions may be used. This list of possible solutions is not anticipated to be all encompassing and many other solution types could be used as will be understood by one skilled in the art. The reasons for performing a wash step include, but are not limited to: causing changes to interactions between target viral, bacterial or other particles and non-target particles present in the sample; solubilizing or washing away of natural organic matter, including humic and fulvic substances, proteins, polysaccharides, and other non-target particles and molecules; improving recovery of target particles by flushing the captured particles with Tween 20, Tween 80, sodium polyphosphate, glycine or other surfactants, proteins, chemical dispersants, or other chemicals that are known to improving recovery of target particles from membrane filters; and simply improving the buffer exchange process performed on the membrane filter by allowing residual liquid on the membrane to be washed away to the permeate.

Following the wash steps, sample labeling, dying or other treatment steps, such as DNase, RNase or intercalating die treatments, can be performed. Each of these steps follows processes similar to the wash steps described above. Labeling or dying steps are generally identical to the wash steps described except that a labeling or dying solution must be pulled from a different solution bottle and through a different distribution valve before entering syringe 3007. Additionally, the incubation period and number times that the treatment solutions are pulled into and through the hollow fiber membrane filters may require adjustment to provide sufficient time to interact with the target particles.

Following the wash step or any sample labeling, dying, or treatment steps performed, the concentrated sample may be eluted or lysis steps can be performed on target materials prior to elution. To perform a lysis step the same process as used for wash steps can be used to introduce a lysis fluid to the lumen of the hollow fiber membrane filters 3026. To perform the lysis step, any residual wash fluid 3055 in syringe 3007 is first pushed to waste by positioning distribution valve 3006 to connect port 3016 to central port 3008 and syringe pump 3007. Syringe pump 3007 is then activated and the remaining wash fluid is pushed out through waste line 3064.

During the first stage of the lysis protocol, permeate valve 3004 and permeate pressure valve 3005 remain closed, allowing the permeate 3066 side of CPT 3001 to be sealed off from atmosphere. Foam elution valve 3032 is closed and remains closed except when wet foam is being dispensed during other operations. The distribution valve 3006 is positioned to connect port 3011 to central port 3008 and syringe pump 3007. Syringe pump 3007 is then activated to draw air from air inlet 3017 through high efficiency particle arresting (HEPA) filter 3018, fluid line 3019, port 3011, central port 3008 and then into the syringe pump 3007. The volume of air pulled is dependent on the lysis process being used with more pulled in if the lysis fluid 3060 is to be pushed out of the tip and into a final sample container or less if it will be used to perform lysis within CPT 3001. The air drawn into the syringe will be used to push the lysis fluid 3060 out of fluid line 3020 and into the internal retentate flow path of CPT 3001 or through CPT 3001 and out CPT inlet 3027.

Rotary distribution valve 3006 is positioned so that port 3012 is connected to central port 3008. Syringe pump 3007 is then used to draw lysis fluid 3060 from solution bottle 3061 through sterile filter 3062, through sample addition fluid line 3063, and then into syringe pump 3007. The syringe pump 3007 draw volume can range from less than 5% to 200% of the total internal volume of hollow fiber membrane filters 3026 in CPT 3001. In the exemplary embodiment the draw volume will range from 10% to 100% of the total internal volume of hollow fiber membrane filters 3026 in CPT 3001.

In the exemplary embodiment, after the lysis fluid 3060 is drawn into syringe pump 3007 distribution valve 3006 is positioned so that syringe pump 3007 and central port 3008 are connected to port 3010. Syringe pump 3007 is then activated to dispense lysis fluid 3060 through fluid line 3020 and into CPT 3001 through CPT retentate port 3002. Lysis fluid 3060 flows through check valve 3025 and down the lumen of the hollow fiber membrane filters 3026 within CPT 3001 and out of CPT opening 3027 and into a sample container. This step may be performed at high flow rate to push target materials into lower sections of the hollow fiber membrane filters 3026 or out of CPT opening 3027 or it may be performed at a slower flow rate to give increased contact time between the bulk lysis fluid 3060 and the target materials and inner surface of the membrane. These processes may be performed with the permeate at atmospheric pressure and sealed closed or with positive pressure on the permeate. The lysis fluid 3060 is pushed out of CPT 3001 using the air in syringe 3070. After pushing the entire volume of lysis fluid 3060 out of CPT 3001 an additional incubation period may be allowed. Generally, this period will range from 10 seconds to 30 minutes, but more specifically from 30 seconds to 15 minutes. The incubation period enables the lysis fluid 3060 to lyse and solubilize materials that may bind to the hollow fiber membrane filters 3026 surface and therefor can be used to improve recovery of target particles while also initiating lysis. Of note, the lysis fluid 3060 may also be heated prior to injection into CPT 3001 using onboard heated lines.

After the lysis incubation period the remaining material in the hollow fiber membrane filters 3026 is flushed out and into the same sample container. The elution process involved flushing down the lumen with carbonated wet foam while the permeate is held at atmospheric pressure and closed off by permeate pressure valve 3005 and permeate valve 3004 or positive permeate pressure is applied as described in the other operations above. When performing elution with a positive permeate pressure the positive pressure is applied to the permeate 3034 immediately before or during the wet foam elution. In this way, minimal permeate fluid is pushed back through the hollow fiber membrane filters 3026 during the process. The application of pressure to the permeate 3066 during the elution process improves recovery efficiency by allowing a small amount of permeate fluid to begin to back flush through the hollow fiber membrane filters 3026 wall.

To perform the elution, the rotary distribution valve 3006 is connected to elution fluid canister 3065 by fluidically connecting central port 3008 to port 3009. Syringe 3007 is then very slowly drawn open until the desire volume of elution fluid is drawn into syringe 3007. Syringe 3007 must be drawn back slowly to reduce the level of off-gassing of carbon dioxide from the elution fluid. After drawing back syringe 3007 to the final position rotary valve 3006 can be turned to a closed-off port and syringe 3007 can be depressed slightly to cause any carbon dioxide gas bubbles to go back into solution. To dispense the foam the rotary valve 3006 is turned to connect central port 3008 to port 3010. This connects syringe 3007 to CPT 3001 through retentate port 3002. The elution fluid can then travel out of syringe 3007 through fluid line 3020 and down the retentate side of hollow fibers 3026 while forming a wet foam that works to sweep captured particles from the retentate and dispense them in a reduced volume from CPT opening 3027. Off gassing in the syringe causes the foam to travel out and the syringe 3007 can also be pushed in to drive the foam and remaining liquid out.

The elution fluid generally contains a buffered solution with a surfactant or other foaming agent and is held under a carbon dioxide head pressure between 30 psi and 250 psi or more preferably between 75 psi and 150 psi. Alternatively, other soluble or insoluble gasses including nitrogen, nitrous oxide and any number of other gases or combination of gasses can be used as will be well known by those skilled in the art. Foaming agents can include surfactants such as Tween 20, Tween 20, Triton X-100, SDS, Pluronic and any number of other surfactants that will be known to those skilled in the art. Additionally, alternative foam agents including proteins, growth media and other materials that will produce a foam can be used along with a surfactant or alone. Buffers, salts, chelators and other additives may also be used to maintain pH, bacterial viability, enhance recovery, improve assay compatibility, or to achieve other results as will be well understood by those skilled in the art.

The elution fluid can also be formulated to act alone as a lysis fluid or to improve lysis when combined with the lysis fluid 3060. As one example lysis fluid 3060 may contain enzymes to initiate breakdown of cell wall components and the elution fluid may contain SDS to complete lysis after an incubation step in lysis fluid 3060.

It is important to note that during the elution process the permeate 3006 may either be returned to atmospheric pressure and sealed off by closing valves 3004 and 3005 or pressure may be applied to permeate 3066 using the backpulsing processes described above. By using permeate pressure flow of elution fluid through pores in the walls of hollow fibers 2926 may be reduced or nearly eliminated or at high permeate pressures a positive flow rate from permeate 3066 to the retentate side of hollow fibers 3026 may be achieved. This reverse flow can enable improved particle recovery in some cases.

In one exemplary embodiment of the subject disclosure presented in FIG. 30 check valve 3025 can be eliminated by keeping a plug of clean fluid inside of fluid line 3020 and the internal volume of retentate port 3002. Additionally, if necessary is some applications the syringe can be used to push a very slow flow rate of clean fluid into the retentate port 3002 during sample processing.

Rotary distribution valves and syringes pumps are used in the subject disclosures presented in FIG. 29 and FIG. 30. The fluidic layout of these subject disclosures can be readily modified to achieve the same results by replacing the rotary distribution valves with fluidic manifolds or tubing assemblies with diaphragm valves, gate valve, ball valves, butterfly valves, check valves, needle valves, pinch valves, microfluidic valves and many other valve types that will be well known by those skilled in the art. Additionally, the syringe pump may be replaced by one or more syringe pumps or one or more of other types of liquid pumps or fluid movers that will be well known to those skilled in the art, including, but not limited to gravity feed, centrifugal pump, positive displacement pump, diaphragm pump, gear pump, progressing cavity pump, piston, pump, jet pump, rotary vane pump, screw pump, axial flow pump, and others.

For the purposes of this disclosure, a permeate chamber is any volume that is formed between a permeate surface of a membrane and a housing of the CPT, and a retentate chamber is any volume that is formed between a retentate surface of a membrane and said housing. For a dual-filter CPT, a retentate chamber may be formed between the retentate surfaces, and a permeate chamber may be formed between each permeate surface and its respective housing. In a hollow fiber filter CPT, the permeate chamber may be formed by the combined volume external to each hollow fiber filter, and the retentate chamber may be formed by the combined inner volume of each hollow fiber filter. In alternative embodiments, the positions and configurations of the permeate and retentate chambers may be reversed.

The foregoing instrumentalities have significant utility in medical, environmental, or security applications. In exemplary embodiments, concentration in the manner described facilitates aerosol sampling for pathogens or bioterrorism threat agents that can withstand being placed in a liquid sample for analysis. A list of such pathogens may be provided, for example, as recognized by the Center for Disease Control (CDC). See TABLE 1 and TABLE 2. See TABLE 3 for agents and sizes. These organisms may be studied using conventional techniques that are facilitated by the concentration of samples as described above.

TABLE 1
CDC CATEGORY A AND B
BIOTERRORISM AGENTS LIST
CATEGORY A
Anthrax (Bacillus anthracis)
Botulism (Clostridium botulinum toxin)
Plague (Yersinia pestis)
Smallpox (Variola major)
Tularemia (Francisella tularensis)
Viral hemorrhagic fevers (filoviruses
[e.g., Ebola, Marburg] and arenaviruses
[e.g., Lassa, Machupo])
CATEGORY B
Brucellosis (Brucella species)
Epsilon toxin of Clostridium perfringens
Food safety threats (e.g., Salmonella
species, Escherichia coli O157:H7,
Shigella)
Glanders (Burkholderia mallei)
Melioidosis (Burkholderia pseudomallei)
Psittacosis (Chlamydia psittaci)
Q fever (Coxiella burnetii)
Ricin toxin from Ricinus communis (castor beans)
Staphylococcal enterotoxin B
Typhus fever (Rickettsia prowazekii)
Viral encephalitis (alphaviruses [e.g.,
Venezuelan equine encephalitis, Eastern
equine encephalitis, Western equine encephalitis])
Water safety threats (e.g., Vibrio cholerae,
Cryptosporidium parvum)

TABLE 2
SECONDARY POTENTIAL
BIOLOGICAL THREAT AGENTS

| | |
|---|---|
| Viri/prions | Histoplasma capsulatum |
| Flaviviruses (Yellow fever virus, West Nile | Cryptococcus neoformans |
| virus, Dengue, Japanese Encephalitis, | Aspergillus niger |
| TBE, etc.) | Pathogenic fungi |
| Hepatitis (A, B, C) | Acremomium spp. |
| Prions (CJD, BSE, CWD) | Alternaria alternate |
| Alphaviruses (VEE, EEE, WEE) | Apophysomyces elegans |
| MERS | Aspergillus terreus |
| SARS | Bipolaris spp. |
| SARS-COV-2 | Bipolaris spicifera |
| COVID-19 | Blastoschizomyces capitatus |
| Nipah virus | Candida krusei |
| Rabies virus | Candida lusitaniae |
| Rhinovirus (could be modified?) | Cladophialophora bantiana |
| Polioviruses | Cunnihamella berholletiae |
| Hantaviruses | Curvularia lunata |
| Filoviruses (Ebola, Marburg, Lassa) | Exserohilum rostratum |
| Bacilli | Fusarium moniliforme |
| Mycobacterium tuberculosis, drug resistant | Fusarium solani |
| Mycobacteria other than TB, like C. leprae | Hansenula anomala |
| Streptococcus pneumoniae | Lasiodilodia theobromae |
| Streptococcus pyogenes | Malassezia furfur |
| Streptococcus aureus | Paecilomyces lilacinus |
| Clostridium tetani | Paecilomyces bariotii |
| Clostridium difficile | Penicillium marneffei |
| Bacillus cereus | Phialemonium curvatum |
| Coxiella brunette (Q fever) | Phialophora parasitica |
| Francisella tularensis | Phialophora richardsiae |
| Borrelia recurrentis | Ramichloridium spp. |
| Rickettsia rickettsii | Rhizomucor pusillus |
| R. prowazekii | Rhizopus rhizopodiformus |
| Shigella sonnei | Rhodotorula rubra |
| Bartonella henselae | Sacchromyces cerevisiae |
| Yersinia enterolitica | Scedosporium prolificans |
| Yersinia pseudotuberculosis | Trichosporon beigelii |
| Neisseria meningitidis | (T. asahii) |
| Legionella pneumophila | Wangiella dermatitidis |
| Burkholderia pseudomallei | |
| Pasturella multocida | |
| Other Pathogenic Microorganisms | |
| Cryptosporidium parvum | |

TABLE 3
PHYSICAL SIZES OF SOME AGENTS AND SURROGATES

| TARGET | PHYSICAL SIZE |
|---|---|
| Bacillus thuringiensis | approximately 1 μm |
| endospore | |
| Bacillus anthracis | approximately 1 μm |
| endospore | |
| Yersinia pestis | Gram negative rod-ovoid 0.5-0.8 μm |
| | in width and 1-3 μm in length |
| Yersinia rohdei | approximately 1 μm |
| Venezuelan Equine | 70 nm (0.07 μm) |
| Encephalitis | |
| Gamma-killed MS2 | 2 mD or about 25 nm (0.025 μm) (but |
| | will pass through a 300 kD pore size |
| | but is retained by a 100 kD pore size |
| | Wick and McCubbin-ECBC) |
| Ovalbumin | 45 kD or 6 nm (0.006 μm) |
| Botulinum Toxoid A | 150 to 900 kD or 10 nm to 70 nm (0.01 |
| | μm to 0.07 μm)(Normally published as |
| | 150 kD however some publications |
| | state that toxoid A can be released as |
| | complexes comprised of the 150 kD |
| | toxin protein along with associated |
| | non-toxin proteins and can therefore |
| | be released in 900 kD, 500 kD, and |
| | 300 kD forms. |
| DNA | 1000 Bp or 600 kD up to 15,000 Bp or |
| | 9 mD |

The concentrating filter tips (CPTs) used in this disclosure may be any disposable filter tip, for instance, a 0.1 micron polyethersulfone filter which is sold by Assignee under part numbers CC8001-10 and CC8001-60, or 0.4 micron polycarbonate track etched membranes that are sold as CC8000-10 or CC8000-60. A flow rate of 100+mL/min is supported, with an input sample volume range of up to 2 L, and a final concentrated sample volume range that is user-selectable from, for instance, 200-1000 μL. Exemplary particle size capabilities are dependent on the CPT used, and can range from 0.1 μm-0.4 μm for bacteria, parasites, molds, spores, and whole cells. Ultrafiltration for virus and free DNA may also be conceivable to those having ordinary skill in the art in light of this disclosure. Further any filter or membrane filter in the standard range of ultrafiltration or microfiltration membrane filters as well as fibrous filters and filters with mechanisms for attraction, such as zeta potential filters, may be used in a CPT device for capture of particles ranging from less than 1 kD molecular weight or less than 0.001 μm to particles or organisms up to as large as 1 mm in diameter. Ultrafiltration membranes in the range of 1 kD to 1,000 kD can be used in CPTs for a variety of concentration applications including proteins and other soluble and insoluble materials and small particles including pyrogens. Free DNA, and free RNA may be captured and concentrated using filters in the approximate range of 0.001 μm to 0.02 μm or 1 kD to 300 kD. Virus may be captured and concentrated using filters generally in the physical or effective pore size range of 0.001 μm to 0.1 μm or in the general molecular weight cut-off range of 1 kD to 1,000 kD. Bacteria can be concentrated using membranes generally in the range of 0.01 to 0.5 μm. Moreover, any membrane with a physical or effective pore size sufficiently small enough to capture the particle of interest may be used and in some instances pore size significantly smaller than the target particle may be selected such that multiple targets, of different sizes may be concentrated into a single concentrated sample. Further, as can be appreciated by someone skilled in the art, novel membranes and filters, and membranes and filters other than those mentioned here, may serve the purpose of retaining certain particles of interest and may provide a reliable filter for use in a CPT.

Moreover, although concentration of bacteria are disclosed, any of the disclosed embodiments may be used to concentrate bacterial pathogens within the blood in exemplary embodiments, after preparation of a blood sample by removal of blood components such as red blood cells, etc. Other applications include food and beverage processing and safety monitoring (of spoilage organisms and pathogens from process waters, liquid samples from food preparation surfaces, product wash waters), environmental monitoring (recreational water monitoring, waste water monitoring, legionella monitoring), drinking water, forensics, pharmaceutical manufacturing, and biodefense.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A device, comprising:
a housing having a top end and a bottom end;
a filter enclosed within the housing, the filter being vertically oriented and spanning a length of the housing, the filter having a retentate surface and an opposing permeate surface;
a retentate port positioned adjacent the top end of the housing;
a permeate port positioned adjacent the top end of the housing;
a sample port positioned adjacent the bottom end of the housing;
a rotary distribution valve connected to the retentate port;
a syringe pump connected to the rotary distribution valve, wherein the syringe pump is adapted to push a volume of fluid that is equivalent to a known internal volume of a retentate portion of the tip; and
a source of addition fluid, a source of blocking fluid, a source of wash fluid; and a source of lysis fluid;
wherein all sources of fluid are connected to separate and distinct ports on the rotary distribution valve.

2. The device in claim 1, further comprising:
a backpressure system for applying backpressure to the filter permeate chamber.

3. The device in claim 1, further comprising:
a source of filter elution fluid; and
a separate valve for controlling the flow of elution fluid through the filter housing and out the sample port.

4. The device in claim 3, wherein the filter elution fluid source is pressurized with a soluble gas.

5. The device in claim 3, wherein the filter elution fluid contains a surfactant or detergent.

6. The device in claim 3, wherein the filter elution fluid contains a buffer.

7. The device in claim 3, wherein the filter elution fluid contains a salt.

8. The device in claim 3, wherein the filter elution fluid contains a dispersant.

9. The device in claim 3, wherein the filter elution fluid contains a chelator.

10. The device in claim 1, further comprising:
a source of filter elution fluid connected to the rotary valve;
wherein the rotary valve and syringe pump are used for dispensing the filter elution fluid.

11. A method, comprising:
inserting a retentate port and permeate port of a housing with an enclosed filter into a port in a sample processing device;
placing a bottom sample port positioned adjacent a bottom end of the housing into a liquid sample;
drawing a sample using a permeate pump, attached to a housing permeate port, through the filter in the housing, capturing particles on a filter surface;
selecting one or more of a sample addition fluid, a blocking fluid, a wash fluid, and a lysis fluid, and pushing a volume of fluid into the filter housing before, during, and after processing the sample using a syringe pump and connected rotary distribution valve;
after processing the sample, pushing a volume of elution fluid through the filter housing to recover particles from the filter surface.

12. The method in claim 11, wherein the rotary valve and syringe are used for dispensing the filter elution fluid.

13. The method in claim 11, wherein a separate valve and timer are used for dispensing the filter elution fluid.

14. The method in claim 11, wherein the blocking fluid contains a surfactant or detergent.

15. The method in claim 11, wherein the blocking fluid contains a dispersant.

16. The method in claim 11, further comprising:
applying positive pressure to a filter permeate chamber within the filter housing using a backpressure system.

17. The method in claim 16, wherein the positive pressure is applied at set intervals.

18. The method in claim 16, wherein the positive pressure is applied when system flowrate drops below a setpoint.

19. The method in claim 11, further comprising:
injecting a small volume of wash fluid into the filter retentate at set intervals during sample processing.

20. The method in claim 19, wherein the wash fluid is injected when the system flowrate drops below a setpoint.
